SEQUENCE LISTING (10 sequences)

SEQ ID NO: 1 - protein, 201 aa, homo sapiens
PKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 201 aa, homo sapiens
PKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 201 aa, homo sapiens
PKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 4 - protein, 201 aa, homo sapiens
PKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 5 - protein, 201 aa, homo sapiens
PKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 204 aa, homo sapiens
SPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 7 - protein, 221 aa, homo sapiens
ALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 8 - protein, 221 aa, homo sapiens
ALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 9 - protein, 223 aa, homo sapiens
SFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 10 - protein, 209 aa, homo sapiens
AVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

(19) United States
(12) Patent Application Publication
(10) Pub. No.: US 2023/0107363 A1
(43) Pub. Date: Apr. 6, 2023

(54) POLYPEPTIDE VARIANTS AND USES THEREOF

(71) Applicant: GENMAB B.V., Utrecht (NL)

(72) Inventors: Rob DE JONG, Utrecht (NL); Frank Beurskens, Utrecht (NL); Marije Overdijk, Utrecht (NL); Kristin STRUMANE, Werkhoven (NL); Janine Schuurman, Diemen (NL); Paul Parren, Odijk (NL)

(21) Appl. No.: 17/745,667

(22) Filed: May 16, 2022

Related U.S. Application Data
(63) Continuation of application No. 16/345,044, filed on Apr. 25, 2019, now abandoned.

(30) Foreign Application Priority Data
Nov. 1, 2016 (DK) PA 2016 00674

(51) Int. Cl. C07K 16/28; A61P 31/10; A61P 35/00; A61P 31/12; A61P 31/04; C07K 16/00; A61P 37/06

(52) U.S. Cl. CPC C07K 16/2878; A61P 31/10; A61P 35/00; A61P 31/12; A61P 31/04; C07K 16/00; A61P 37/06; C07K 16/2896; C07K 16/2887; C07K 16/2893; C07K 2317/75; C07K 2317/73; C07K 2317/92; C07K 2317/734; C07K 2317/72; C07K 2317/524; C07K 2317/71; C07K 2317/732; C07K 2317/528; C07K 2317/94; C07K 2317/522; C07K 2317/526

(57) ABSTRACT

Described herein are polypeptides and antibodies comprising a variant Fc region. The variant Fc region provides for stabilized Fc-Fc interactions when the polypeptide(s), antibody or antibodies are bound to its target, antigen or antigens on the surface of a cell, while at the same time also having decreased complement-dependent cytotoxicity (CDC) and may also have decreased activation of other effector functions resulting from one or more amino acid modifications in the Fc region.

### CROSS-REFERENCE TO RELATED APPLICATIONS

This application is a continuation of U.S. application Ser. No. 16/345,044, filed Apr. 25, 2019, which is a 35 U.S.C. 371 national stage filing of International Application No. PCT/EP2017/077971, filed Nov. 1, 2017, which claims priority to Danish Patent Application No. PA 2016 00674, filed Nov. 1, 2016. The contents of the aforementioned applications are hereby incorporated by reference.

### SEQUENCE LISTING

The instant application contains a Sequence Listing which has been submitted electronically in ASCII format and is hereby incorporated by reference in its entirety. Said ASCII copy, created on May 16, 2022, is named GMI-179USCN_SequenceListing_2022-05-16.txt and is 18,799 bytes in size.

### FIELD OF THE INVENTION

The present invention concerns Fc region-containing polypeptides, such as antibodies, that have decreased Fc effector functions such as, decreased binding to C1q, decreased complement-dependent cytotoxicity (CDC) and may also have decreased activation of other effector functions resulting from one or more amino acid modifications in the Fc-region.

### BACKGROUND OF THE INVENTION

Fc-mediated effector functions of monoclonal antibodies, such as complement-dependent cytotoxicity (CDC), antibody-dependent cell-mediated cytotoxicity (ADCC) and antibody-dependent cell-mediated phagocytosis (ADCP) contribute to the therapeutic window defined by efficacy and toxicity. CDC is initiated by binding of C1q to the Fc regions of antibodies. C1q is a multimeric protein consisting of six globular binding heads attached to a stalk. The individual globular binding heads have low affinity for IgG; and C1q must gain avidity by binding multiple IgG1 molecules on a cell surface to trigger the classical complement pathway. ADCC and ADCP are initiated by binding of the IgG Fc region to Fcγ receptors (FcγR) on effector cells.

IgG hexamerization upon target binding on the cell surface has been shown to support avid C1q binding. The hexamerization is mediated through intermolecular non-covalent Fc-Fc interactions, and Fc-Fc interactions can be enhanced by point mutations in the CH3 domain, including E345R and E430G.

WO2013/004842 discloses antibodies or polypeptides comprising variant Fc regions having one or more amino acid modifications resulting in modified effector functions such as complement-dependent cytotoxicity (CDC).

WO2014/108198 discloses polypeptides such as antibodies comprising variant Fc regions having one or more amino acid modifications resulting in increased complement-dependent cytotoxicity (CDC).

WO2012/130831 concerns Fc region-containing polypeptides that have altered effector function as a consequence of one or more amino acid substitutions in the Fc region of the polypeptide. These polypeptides exhibit reduced affinity to the human FcγRIIIa and/or FcγRIIa and/or FcγRI compared to a polypeptide comprising the wildtype IgG Fc region, and exhibit reduced ADCC induced by said polypeptide to at least 20% of the ADCC induced by the polypeptide comprising a wild-type human IgG Fc region. WO2012/130831 does not disclose Fc region-containing polypeptides which have enhanced Fc-Fc interactions and/or enhanced ability to form hexamers.

As described above, previous efforts in enhancing Fc-Fc interactions between polypeptides and/or antibodies have the effect of enhancing effector functions such as enhanced CDC and or ADCC, which leads to cell death of the target cell to which the antibody or polypeptide binds.

Enhanced Fc-Fc interactions between antibodies can be used to amplify the effect of the antibody binding to its target on a cell surface, but in instances where the target cell is an effector cell such as a T cell, NK cell or other effector cells where the mechanism of action involves binding to an effector cell (e.g. such as in a bispecific antibody), then the interaction with C1q or Fc-gammaR and/or activation of Fc effector functions such as CDC and/or ADCC may be unwanted. Therefore there is a need for antibodies with enhanced Fc-Fc interactions, but that does not engage C1q binding and/or have Fc-gammaR interactions and thereby activate Fc effector functions such as CDC and/or ADCC.

Accordingly, it is an object of the present invention to provide a variant polypeptide or antibody comprising an Fc region of a human IgG and an antigen binding region, which polypeptide has increased Fc-Fc interactions and reduced effector functions such as CDC and/or ADCC compared to a parent polypeptide, where the parent polypeptide is a human IgG of the same isotype and having the same antigen binding region, with a first mutation which is an Fc-Fc enhancing mutation in an amino acid position corresponding to E345, E430 or S440 in human IgG1, with the proviso that the mutation in position S440 is S440Y or S440W.

It is another object of the present invention to provide a polypeptide or an antibody with enhanced Fc-Fc interaction properties without inducing effector functions such as CDC. It is another object of the present invention to provide a polypeptide or an antibody with enhanced Fc-Fc interaction properties without inducing effector functions such as ADCC. It is another object of the present invention to provide a polypeptide or an antibody with enhanced Fc-Fc interaction properties without inducing effector functions such as CDC and ADCC. It is a further object of the present invention to provide for a polypeptide or an antibody with enhanced Fc-Fc interactions while having decreased Fc effector functions such as decreased CDC and/or ADCC compared to a parent polypeptide with only a first mutation which results in enhanced Fc-Fc interactions. It is yet another object of the present invention to provide for a polypeptide or an antibody that activates signaling, optionally induces enhanced signaling, when the antigen binding region of the polypeptide or antibody is bound to the corresponding antigen without activating Fc effector functions such as CDC and/or ADCC.

### SUMMARY OF THE INVENTION

In a first aspect, the invention provides for polypeptides or antibodies having an Fc region and an antigen binding region where the Fc region has a first mutation which is an Fc-Fc enhancing mutation and a second mutation which decreases C1q binding and/or FcgammaR binding and/or Fc effector functions such as CDC and/or ADCC activity.

The inventors of the present invention surprisingly found that by introducing a second mutation in the Fc region corresponding to amino acid position E322 or P329 in the Fc region of a human IgG, the oligomerization capability of the first mutation could be maintained while effector functions such as, CDC, and/or ADCC activity were decreased.

Without being limited to theory, it is believed that the polypeptides or antibodies of the invention are capable of a more stable binding interaction between the Fc regions of two polypeptides or antibody molecules when bound to the target on a cell surface, which leads to an enhanced oligomerization, such as hexamer formation, without enhancing Fc mediated effector functions. The polypeptides or antibodies of the invention further have decreased C1q binding and/or decreased FcgammaR binding compared to their parent polypeptide or parent antibody which comprises a first mutation but not a second mutation. The polypeptides or antibodies of the invention have decreased Fc effector functions compared to their parent polypeptide or parent antibody which comprises a first mutation but not a second mutation. Some polypeptides or antibodies of the invention have a decreased Fc effector function such as CDC compared to a parent polypeptide or parent antibody. Some polypeptides or antibodies of the invention have a decreased Fc effector function such as ADCC compared to a parent polypeptide or parent antibody. Some polypeptides or antibodies of the invention have a decreased Fc effector function such as CDC and ADCC compared to a parent polypeptide or parent antibody Some polypeptides or antibodies of the invention further have a decreased Fc effector response compared to an identical polypeptide or antibody which does not comprise a first and a second mutation, i.e. a wild type Fc region. Some polypeptides of the invention have reduced C1q binding and/or reduced FcgammaR binding. Some polypeptides or antibodies of the invention have a reduced CDC response. Some polypeptides or antibodies of the invention have a reduced ADCC response. Some polypeptides or antibodies of the invention are characterized by having both a reduced ADCC and CDC response, and/or other reduced effector responses.

In one aspect, the present invention provides for a polypeptide or an antibody comprising an Fc region of a human IgG and an antigen binding region, wherein the Fc region comprises a CH2 and CH3 domain, said Fc region comprising a (i) first mutation and a (ii) second mutation corresponding to the following amino acid positions in human IgG1 according to EU numbering (Edelman et al., Proc Natl Acad Sci USA. 1969 May; 63(1):78-85; Kabat et al., Sequences of Proteins of Immunological Interest, Fifth Edition. 1991 NIH Publication No. 91-3242):
i. first mutation at E430, E345 or S440, with the proviso that the mutation in S440 is S440Y or S440W; and
ii. second mutation at K322 or P329.

That is, the inventors of the present invention in a first aspect of the invention found that introducing a second mutation in one of the amino acid positions corresponding to K322 or P329 in the Fc region of a polypeptide or an antibody having a first mutation, where the first mutation enhances Fc-Fc interactions and thus enhanced oligomerization upon target binding, the second mutation was able to reduce Fc effector functions. The mutation corresponding to amino acid position K322 or P329 in the Fc region of a polypeptide or an antibody has the effect of reducing one or more Fc effector functions to a level that is decreased compared to a parent polypeptide or parent antibody having the identical first mutation, but not the second mutation. Thus, in one embodiment of the invention the polypeptide or antibody has at least one first mutation which may be selected from one of the following positions E430, E345 or S440, with the proviso that the mutation in S440 is S440Y or S440W, and the polypeptide or antibody has at least one second mutation which may be selected from one of the following positions K322 or P329.

In one embodiment of the present invention, the first mutation is selected from the group consisting of: E430G, E345K, E430S, E430F, E430T, E345Ω, E345R, E345Y, S440W and S440Y. In one embodiment of the present invention, the first mutation is selected from E430G or E345K. In a preferred embodiment the first mutation is E430G.

In one embodiment of the present invention, the second mutation is selected from the group consisting of: K322E, K322D, K322N, P329H, P329K, P329R, P329D, P329E, P329F, P329G, P329I, P329L, P329M, P329N, P329Q, P329S, P329T, P329V, P329W, P329A and P329Y.

In one embodiment of the present invention, the second mutation is at amino acid position P329, with the proviso that the second mutation is not P329A.

In one embodiment of the present invention, the second mutation is at amino acid position P329, with the proviso that the second mutation is not P329A or P329G.

In one embodiment of the present invention, the Fc region does not comprise a mutation in the amino acid positions corresponding to L234 and L235. That is, in one embodiment of the present invention the Fc region comprises the wild type amino acids L and L in the positons corresponding to L234 and L235 in human IgG1, wherein the positions are according to EU numbering.

In a further aspect, the present invention relates to a method of decreasing an Fc effector function of a polypeptide or antibody comprising an Fc region of a human IgG and an antigen binding region, wherein the Fc region comprises a CH2 and CH3 domain with a (i) first mutation corresponding to the following amino acid positions in human IgG1 according to EU numbering: E430, E345 or S440, with the proviso that the mutation in S440 is S440Y or S440W, which method comprises introducing a (ii) second mutation corresponding to the following amino acid positions in human IgG1 according to EU numbering: K322 or P329.

That is, the inventors of the present invention found that by introducing a second mutation in one of the amino acid positions corresponding to K322 or P329 of a polypeptide or antibody having a first mutation corresponding to one of the amino acid positions E430, E345 or S440, with the proviso that the mutation in S440 is S440Y or S440W, which leads to enhanced oligomerization upon target binding on a cell surface and enhanced Fc effector functions, one or more of the effector functions could be decreased. Hence, the second mutation may decrease the Fc effector function of a polypeptide or antibody to a level that is comparable to, or less than, the level of a parent polypeptide with a first mutation at a position corresponding to E430, E345 or S440, with the proviso that the mutation in S440 is S440Y or S440W.

In another aspect, the present invention relates to a composition comprising at least one polypeptide or antibody as described herein.

In another aspect, the present invention relates to a polypeptide, antibody or a composition as described herein for use as a medicament.

In another aspect, the present invention relates to a polypeptide, antibody or a composition as described herein for use in the treatment of cancer, autoimmune disease, inflammatory disease or infectious disease.

In another aspect, the present invention relates to a method of treating an individual having a disease comprising administering to said individual an effective amount of a polypeptide, an antibody or composition as described herein.

These and other aspects of the invention, particularly various uses and therapeutic applications for the polypeptide or antibody, are described in further detail below.

### BRIEF DESCRIPTION OF THE DRAWINGS

FIG. 1 shows the effect of C1q binding inhibition mutations (D270A/K322A indicated as AA) in the Fc domain of IgG1 on the CDC efficacy of IgG-005 variants with and without mutation(s) for enhanced Fc-Fc interactions (E430G indicated as G; E345R indicated as R, E345R/E430G/S440Y indicated as RGY). CD38-positive Daudi cells were incubated with concentration series of CD38 (mutant) antibody in the presence of 20% pooled normal human serum (NHS). CDC efficacy is presented as the percentage lysis determined by the percentage propidium iodide (PI)-positive cells. The IgG1-b12 mAb against HIV gp120, and mutants thereof were used as a non-binding isotype control mAb. Representative examples are shown.

FIGS. 2A and 2B show the effect of the single amino acid substitutions in the human IgG1 C1q binding site on the CDC efficacy of IgG-005 variants with the E430G mutation for enhanced Fc-Fc interactions. (FIG. 2A) For the CDC assay, Daudi cells were incubated with a concentration series of IgG1-005-E430G with the D270R, K322E, P329D or P329R mutation in the presence of 20% pooled NHS. CDC efficacy is presented as the percentage lysis determined by the percentage propidium iodide (PI)-positive cells. A sample without antibody was used as a negative control for CDC efficacy. A representative example of 2 experiments is shown. (FIG. 2B) Binding of C1q to cell-bound IgG1-005-E430G antibodies with the K322E, P329D or P329R mutation was analyzed by flowcytometric analysis on FACS and represented by the mean fluorescence intensity (MFI) of FITC-labelled rabbit-anti-HuC1q antibody.

FIG. 3 shows the effect of substituting amino acid K322 on the CDC efficacy of IgG-005-E430G with enhanced Fc-Fc interactions. Daudi cells were incubated with a concentration series of the CD38 antibody variants in the presence of 20% pooled NHS. CDC efficacy is presented as the percentage lysis determined by the percentage propidium iodide (PI)-positive cells. Antibody IgG1-b12-E430G against HIV gp120 was used as a non-binding isotype control with Fc-Fc enhancing mutation.

FIGS. 4A and 4B show the biophysical characterization of IgG1-005-E430G antibody variants with the additional mutation K322D, K322E or K322N by capillary electrophoresis sodium dodecyl sulfate (CE-SDS) in (FIG. 4A) and high-performance size exclusion chromatography (HP-SEC) in (FIG. 4B). (FIG. 4A) Left panel: non-reducing conditions; right panel: reducing conditions. (FIG. 4B). HP-SEC profiles of individual antibodies were plotted staggered with a Y-offset of 0.1 A280 units.

FIG. 5 shows the effect of substituting amino acid P329 on the CDC efficacy of IgG-005-E430G with enhanced Fc-Fc interactions. Daudi cells were incubated with a concentration series of the CD38 antibodies in the presence of 20% pooled normal human serum (NHS). CDC efficacy is presented as the percentage lysis determined by the percentage propidium iodide (PI)-positive cells. Antibody IgG1-b12-E430G against HIV gp120 was used as a non-binding isotype control with Fc-Fc enhancing mutation.

FIG. 6 shows the effect of substituting amino acid P329 on FcγRIIIa activation by IgG-005-E430G with enhanced Fc-Fc interactions as determined in a Bioluminescent ADCC Reporter BioAssay. FcγRIIIa activation by antibodies bound to Daudi cells was quantified using FcγRIIIa-expressing Jurkat reporter cells that express luciferase upon FcγRIIIa binding. The production of luciferase is presented by relative luminescence units (RLU). For each data point, the mean and standard deviation of duplicates is presented. A representative example of two experiments is shown.

FIG. 7 shows the effect of mutations K322E, P329A, P329D, P329K and P329R on the ADCC-mediated killing by IgG1-005-E430G. ADCC of Daudi cells was determined in an in vitro ⁵¹Cr-release assay with freshly isolated PBMC from healthy human donors at an E:T ratio 100:1. Antibody IgG1-b12 against HIV gp120 was used as a non-binding isotype control. For each data point, the mean and standard deviation of 5 replicate samples is presented. A representative example with PBMC of one donor is shown.

FIGS. 8A and 8B show the effect of introducing the P329D mutation on C1q binding or the CDC efficacy of different variants of IgG-005 with enhanced Fc-Fc interactions (E345K, E345R and E345R/E430G/S440Y indicated as RGY). (FIG. 8A) C1q binding to cell-bound antibodies was analyzed by flow cytometric analysis on FACS and represented by the mean fluorescence intensity (MFI) of FITC-labelled rabbit-anti-HuC1q antibody. (FIG. 8B) An in vitro CDC assay on Daudi cells was performed in the presence of 20% pooled normal human serum (NHS). CDC efficacy is presented as the percentage lysis determined by the percentage propidium iodide (PI)-positive cells. Antibody IgG1-b12 against HIV gp120 was used as a non-binding isotype control.

FIGS. 9A-9C show the biophysical characterization of IgG1-005-RGY antibody variants with the additional mutation K322E or P329D by HP-SEC in (FIG. 9A), CE-SDS in (FIG. 9B) and native MS in (FIG. 9C).

FIGS. 10A-10C show the effect of the P329D and K322E mutation on Fc-Fc interactions and clustering of saturating concentrations of agonistic DR5 antibodies with the E430G mutation for enhanced Fc-Fc interactions. (FIG. 10A) The involvement of Fc-Fc interactions in the induction of apoptosis by agonistic DR5 antibodies with the E430G mutation is shown in a 3-days viability assay on BxPC-3 human cancer cells with inhibition of killing in the presence of the Fc-binding peptide DCAWHLGELVWCT. Introduction of the P329D (FIG. 10B) or K322E (FIG. 10C) mutation reduced the IC50 on killing by agonistic DR5 antibodies with the E430G mutation, but maximum kill was still achieved, as shown in a 3-days viability assay on BxPC-3 human cancer cells at saturating antibody concentrations of 5 μg/mL (FIG. 10B) and 10 μg/mL (FIG. 10C). Error bars indicate standard deviation.

FIGS. 11A and 11B show the clearance rate of 500 μg i.v. administered antibody in SCID mice. (FIG. 11A) Total human IgG in serum samples was determined by ELISA and plotted in a concentration versus time curve. Each data point represents the mean+/−standard deviation of triplicate samples. (FIG. 11B) Clearance until day 21 after administration of the antibody was determined following the formula D*1.000/AUC with D, injected dose and AUC, area under the curve of the concentration-time curve. A representative example of two independent ELISA experiments is shown.

FIG. 12 shows the effect of substituting amino acid P329 on the CDC efficacy of IgG1-005-E430G with enhanced Fc-Fc interactions. Daudi cells were incubated with a concentration series of the CD38 antibodies in the presence of 20% pooled normal human serum (NHS). CDC efficacy is presented as the percentage lysis determined by the percentage propidium iodide (PI)-positive cells. Antibody IgG1-b12 against HIV gp120 was used as a non-binding isotype control.

FIG. 13 shows the effect of substituting amino acid P329 on the CDC efficacy of different IgG isotype variants of Campath-E430G with enhanced Fc-Fc interactions. Wien 133 cells were incubated with a concentration series of the CD52 antibodies in the presence of 20% pooled normal human serum (NHS). CDC efficacy is presented as the area under dose-response curves, normalized relative to non-binding control antibody IgG1-b12 (0%) and IgG1-Campath (100%).

FIG. 14 shows the effect of substituting amino acid K322 on the CDC efficacy of IgG isotype variants of Campath-E430G with enhanced Fc-Fc interactions. Wien 133 cells were incubated with a concentration series of the CD52 antibodies in the presence of 20% pooled normal human serum (NHS). CDC efficacy is presented as the area under dose-response curves, normalized relative to non-binding control antibody IgG1-b12 (0%) and IgG1-Campath (100%).

FIG. 15 shows the effect of substituting amino acid P329 (top) or K322 (bottom) on the CDC efficacy of IgG1-Campath variants with different Fc-Fc interaction enhancing mutations. Wien 133 cells were incubated with a concentration series of the CD52 antibodies in the presence of 20% pooled normal human serum (NHS). CDC efficacy is presented as the area under dose-response curves, normalized relative to non-binding control antibody IgG1-b12 (0%) and IgG1-Campath (100%).

FIG. 16 shows the effect of substituting amino acid K322 or P329 on the CDC efficacy of anti-CD20 antibodies with enhanced Fc-Fc interactions. Wien 133 cells were incubated with a concentration series of the CD20 antibodies in the presence of 20% pooled normal human serum (NHS). CDC efficacy is presented as the percentage lysis determined by the percentage propidium iodide (PI)-positive cells. Antibody IgG1-b12 was used as a non-binding isotype control.

FIGS. 17A-17F show the effect of substituting amino acid K322 or P329 on the FcγR binding of anti-CD38 IgG1-005 antibodies with E430G-enhanced Fc-Fc interactions measured by ELISA. A concentration series of the indicated antibodies was captured on the wells of a microtiter plate and incubated with a fixed concentration FcγRIIA, FcγRIIB or FcγRIII, or added to wells coated with FcγRI. Variants P329D-E430G, P329K-E430G and P329R-E430G reduced FcγRI binding to background levels; K322E-E430G retained binding to all tested FcγR variants similar to wild type (WT) IgG1-005. Variants L234A/L235A/P329G/E430G (AAGG) and L234F/L235E/P329D/E430G (FEDG) reduced binding of all tested FcγR variants to background levels.

FIG. 18 shows the effect of substituting amino acid P329 on the CDC efficacy of IgG1-Campath or IgG1-11B8 variants with an Fc-Fc interaction enhancing mutation. Wien 133 cells were incubated with a concentration series of mixtures of CD20 and CD52 antibodies in the presence of 20% pooled normal human serum (NHS). CDC efficacy is presented as (top panel) percentage lysis determined by the percentage propidium iodide (PI)-positive cells and (bottom panel) the area under the dose response-response curves, normalized relative to non-binding control antibody IgG1-b12 (0%) and the mixture of IgG1-Campath-E430G+IgG1-11B8-E430G (100%).

FIG. 19 shows the effect of substituting amino acid K322 on the CDC efficacy of IgG1-Campath or IgG1-11B8 variants with an Fc-Fc interaction enhancing mutation. Wien 133 cells were incubated with a concentration series of mixtures of CD20 and CD52 antibodies in the presence of 20% pooled normal human serum (NHS). CDC efficacy is presented as (top panel) percentage lysis determined by the percentage propidium iodide (PI)-positive cells and (bottom panel) the area under the dose response-response curves, normalized relative to non-binding control antibody IgG1-b12 (0%) and the mixture of IgG1-Campath-E430G+IgG1-11B8-E430G (100%).

FIG. 20 shows the effect of substituting amino acids K322, K439, and S440 on CDC efficacy by IgG1-Campath or IgG1-11B8 variants with an Fc-Fc interaction enhancing mutation. Daudi, Raji, Ramos, REH, U266B1, U-698-M, and Wien 133 cells were incubated with 30.0 μg/mL of CD20 and CD52 antibodies as single agents or mixtures in the presence of 20% pooled normal human serum (NHS). CDC efficacy is presented as percentage lysis determined by the percentage propidium iodide (PI)-positive cells normalized relative to non-binding control antibody IgG1-b12 (0%) and either IgG1-Campath-E430G (100%, for REH, U266B1, and Wien 133 cells) or IgG1-11B8-E430G (100%, for Daudi, Raji, Ramos, and U-698-M cells) depending on which antibody induced the highest lysis. EGE=K322E/E430G/K439E; EGK=K322E/E430G/S440K.

FIGS. 21A-21E show the effect of substituting amino acid K322 or P329 on the relative OX40 response of IgG1-SF2 variants with Fc-Fc-enhancing mutation E345R. Thaw-and-Use GloResponse NFκB-luc2/OX40 Jurkat cells were incubated for 5 hours with 2.5 μg/mL antibody in the presence of 5% serum (final) from different sources. OX40 assay responses were recorded by luminescence detected after stimulation of OX40 by anti-OX40 antibodies or 1.5 μg/mL OX40 ligand, which induce the expression of a luciferase reporter gene. Luminescence signals were normalized relative to the responses measured for control incubations without antibody (0%) and with OX40 ligand (100%). FBS: fetal bovine serum; NHS: normal human serum; WT: wild type IgG1-SF2 reference antibody.

FIG. 22: Sequence alignment of the human IgG1, IgG1f, IgG2, IgG3, IgG4, IgA1, IgA2, IgD, IgE and IgM Fc segments corresponding to residues P247 to K447 in the IgG1 heavy chain, using Clustal 2.1 software, as numbered by the EU numbering as set forth in Kabat. The sequences shown represent residues 130 to 330 of the human IgG1 heavy chain constant region (SEQ ID NO:1; UniProt accession No. P01857) and of the allotypic variant IgG1m(f); residues 126 to 326 of the IgG2 heavy chain constant region (SEQ ID NO:2; UniProt accession No. P01859); and residues 177 to 377 of the IgG3 heavy chain constant region (SEQ ID NO:2; UniProt accession No. P01860); and residues 127 to 327 of the IgG4 heavy chain constant region (SEQ ID NO:4; UniProt accession No. P01861); and residues 225-428 of the IgE constant region (Uniprot accession No. P01854); and residues 133-353 of the IgA1 constant region (Uniprot accession No. P01876); and residues 120-340 of the IgA2 constant region (Uniprot accession No. P01877); and residues 230-452 of the IgM constant region (Uniprot accession No. P01871);—and residues 176-384 of the IgD constant region (Uniprot accession No. P01880).

### DETAILED DESCRIPTION OF THE INVENTION

In describing the embodiments of the invention, specific terminology will be resorted to for the sake of clarity. However, the invention is not intended to be limited to the specific terms so selected, and it is understood that each specific term includes all technical equivalents which operate in a similar manner to accomplish a similar purpose.

### Definitions

The term “parent polypeptide” or “parent antibody”, is to be understood as a polypeptide or antibody, which is identical to a polypeptide or antibody according to the invention, but where the parent polypeptide or parent antibody has a first mutation which is an Fc-Fc enhancing mutation e.g. in position E345, E430 or S440 and as a result thereof increased Fc-Fc-mediated oligomerization, increased Fc effector function such as CDC and may also have other enhanced effector functions.

The term “polypeptide comprising an Fc-region of an immunoglobulin and a binding region” refers in the context of the present invention to a polypeptide which comprises an Fc-region of an immunoglobulin and a binding region which is capable of binding to any molecule, such as a polypeptide, e.g. present on a cell, bacterium, or virion. The Fc-region of an immunoglobulin is defined as the fragment of an antibody which would be typically generated after digestion of an antibody with papain (which is known for someone skilled in the art) which includes the two CH2-CH3 regions of an immunoglobulin and a connecting region, e.g. a hinge region. The constant domain of an antibody heavy chain defines the antibody isotype, e.g. IgG1, IgG2, IgG3, IgG4, IgA1, IgA2, IgM, IgD, or IgE. The Fc-region mediates the effector functions of antibodies with cell surface receptors called Fc receptors and proteins of the complement system. The binding region may be a polypeptide sequence, such as a protein, protein ligand, receptor, an antigen-binding region, or a ligand-binding region capable of binding to a cell, bacterium, or virion. If the binding region is e.g. a receptor, the “polypeptide comprising an Fc-region of an immunoglobulin and a binding region” may have been prepared as a fusion protein of Fc-region of an immunoglobulin and said binding region. If the binding region is an antigen-binding region the “polypeptide comprising an Fc-domain of an immunoglobulin and a binding region” may be an antibody, like a chimeric, humanized, or human antibody or a heavy chain only antibody or a ScFv-Fc-fusion. The polypeptide comprising an Fc-region of an immunoglobulin and a binding region may typically comprise a connecting region, e.g. a hinge region, and two CH2-CH3 region of the heavy chain of an immunoglobulin, thus the “polypeptide comprising a Fc-region of an immunoglobulin and a binding region” may be a “polypeptide comprising at least an Fc-region of an immunoglobulin and a binding region”. The term “Fc-region of an immunoglobulin” means in the context of the present invention that a connecting region, e.g. hinge depending on the subtype of antibody, and the CH2 and CH3 region of an immunoglobulin are present, e.g. a human IgG1, IgG2, IgG3, IgG4, IgD, IgA1, IgGA2, IgM, or IgE. The polypeptide is not limited to human origin but can be of any origin, such as e.g. mouse or cynomolgus origin.

The term “Fc-region”, “Fc region”, “Fc-domain” and “Fc domain”, as used herein is intended to refer to the fragment crystallizable region of an antibody. The different terms may be used interchangeably and constitute the same meaning and purpose with respect to any aspect or embodiment of the present invention. The term “parent polypeptide” or “parent antibody”, is to be understood as a polypeptide or antibody, which is identical to a polypeptide or antibody according to the invention, but where the parent polypeptide or parent antibody is without a second mutation, but does have a first mutation which is an Fc-Fc enhancing mutation e.g. in position E345, E430 or S440 and as a result thereof the parent polypeptide or parent antibody has increased Fc-Fc-mediated oligomerization, increased Fc effector function such as CDC and may also have other enhanced effector functions. As indicated above, unless otherwise stated or clearly contradicted by the context, the term “parent polypeptide” or “parent antibody” refers to a polypeptide or antibody with a first Fc-Fc enhancing mutation, but not a second mutation decreasing Fc effector function(s). A polypeptide or antibody accordingly comprises one or more mutations as compared to a “parent polypeptide” or a “parent antibody”.

The term “hinge region” as used herein is intended to refer to the hinge region of an immunoglobulin heavy chain. Thus, for example the hinge region of a human IgG1 antibody corresponds to amino acids 216-230 according to the EU numbering.

The term “CH2 region” or “CH2 domain” as used herein is intended to refer the CH2 region of an immunoglobulin heavy chain. Thus, for example the CH2 region of a human IgG1 antibody corresponds to amino acids 231-340 according to the EU numbering. However, the CH2 region may also be any of the other subtypes as described herein.

The term “CH3 region” or “CH3 domain” as used herein is intended to refer the CH3 region of an immunoglobulin heavy chain. Thus, for example the CH3 region of a human IgG1 antibody corresponds to amino acids 341-447 according to the EU numbering. However, the CH3 region may also be any of the other subtypes as described herein.

The term “immunoglobulin” refers to a class of structurally related glycoproteins consisting of two pairs of polypeptide chains, one pair of light (L) low molecular weight chains and one pair of heavy (H) chains, all four potentially inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as VH) and a heavy chain constant region. The heavy chain constant region typically is comprised of three domains, CH1, CH2, and CH3. The heavy chains are inter-connected via disulfide bonds in the so-called “hinge region”. Each light chain typically is comprised of a light chain variable region (abbreviated herein as VL) and a light chain constant region. The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901 917 (1987)). Unless otherwise stated or contradicted by context, reference to amino acid positions in the constant region in the present invention is according to the EU-numbering (Edelman et al., Proc Natl Acad Sci USA. 1969 May; 63(1):78-85; Kabat et al., Sequences of proteins of immunological interest. 5th Edition—1991 NIH Publication No. 91-3242).

The term “antibody” (Ab) in the context of the present invention refers to an immunoglobulin molecule, a fragment of an immunoglobulin molecule, or a derivative of either thereof, which has the ability to specifically bind to an antigen. The antibody of the present invention comprises an Fc-domain of an immunoglobulin and an antigen-binding region. An antibody generally contains two CH2-CH3 regions and a connecting region, e.g. a hinge region, e.g. at least an Fc-domain. Thus, the antibody of the present invention may comprise an Fc region and an antigen-binding region. The variable regions of the heavy and light chains of the immunoglobulin molecule contain a binding domain that interacts with an antigen. The constant or “Fc” regions of the antibodies may mediate the binding of the immunoglobulin to host tissues or factors, including various cells of the immune system (such as effector cells) and components of the complement system such as C1q, the first component in the classical pathway of complement activation. An antibody may also be a multispecific antibody, such as a bispecific antibody or similar molecule. The term “bispecific antibody” refers to an antibody having specificities for at least two different, typically non-overlapping, epitopes. Such epitopes may be on the same or different targets. If the epitopes are on different targets, such targets may be on the same cell or different cells or cell types. As indicated above, unless otherwise stated or clearly contradicted by the context, the term antibody herein includes fragments of an antibody which comprise at least a portion of an Fc-region and which retain the ability to specifically bind to the antigen. Such fragments may be provided by any known technique, such as enzymatic cleavage, peptide synthesis and recombinant expression techniques. It has been shown that the antigen-binding function of an antibody may be performed by fragments of a full-length antibody. Examples of binding fragments encompassed within the term “Ab” or “antibody” include, without limitation, monovalent antibodies (described in WO2007059782 by Genmab); heavy-chain antibodies, consisting only of two heavy chains and naturally occurring in e.g. camelids (e.g., Hamers-Casterman (1993) Nature 363:446); ThioMabs (Roche, WO2011069104), strand-exchange engineered domain (SEED or Seed-body) which are asymmetric and bispecific antibody-like molecules (Merck, WO2007110205); Triomab (Pharma/Fresenius Biotech, Lindhofer et al. 1995 J Immunol 155:219; WO2002020039); FcAAdp (Regeneron, WO2010151792), Azymetric Scaffold (Zymeworks/Merck, WO2012/058768), mAb-Fv (Xencor, WO2011/028952), Xmab (Xencor), Dual variable domain immunoglobulin (Abbott, DVD-Ig, U.S. Pat. No. 7,612,181); Dual domain double head antibodies (Unilever; Sanofi Aventis, WO20100226923), Di-diabody (ImClone/Eli Lilly), Knobs-into-holes antibody formats (Genentech, WO9850431); DuoBody (Genmab, WO 2011/131746); Bispecific IgG1 and IgG2 (Pfizer/Rinat, WO11143545), DuetMab (MedImmune, US2014/0348839), Electrostatic steering antibody formats (Amgen, EP1870459 and WO 2009089004; Chugai, US201000155133; Oncomed, WO2010129304A2); bispecific IgG1 and IgG2 (Rinat neurosciences Corporation, WO11143545), CrossMAbs (Roche, WO2011117329), LUZ-Y (Genentech), Biclonic (Merus, WO2013157953), Dual Targeting domain antibodies (GSK/Domantis), Two-in-one Antibodies or Dual action Fabs recognizing two targets (Genentech, NovImmune, Adimab), Cross-linked Mabs (Karmanos Cancer Center), covalently fused mAbs (AIMM), CovX-body (CovX/Pfizer), FynomAbs (Covagen/Janssen ilag), DutaMab (Dutalys/Roche), iMab (MedImmune), IgG-like Bispecific (ImClone/Eli Lilly, Shen, J., et al. J Immunol Methods, 2007. 318(1-2): p. 65-74), TIG-body, DIG-body and PIG-body (Pharmabcine), Dual-affinity retargeting molecules (Fc-DART or Ig-DART, by Macrogenics, WO/2008/157379, WO/2010/080538), BEAT (Glenmark), Zybodies (Zyngenia), approaches with common light chain (Crucell/Merus, U.S. Pat. No. 7,262,028) or common heavy chains (κλBodies by NovImmune, WO2012023053), as well as fusion proteins comprising a polypeptide sequence fused to an antibody fragment containing an Fc-domain like scFv-fusions, like BsAb by ZymoGenetics/BMS, HERCULES by Biogen Idec (US00/7951918), SCORPIONS by Emergent BioSolutions/Trubion and Zymogenetics/BMS, Ts2Ab (MedImmune/AZ (Dimasi, N., et al. J Mol Biol, 2009. 393(3): p. 672-92), scFv fusion by Genetech/Roche, scFv fusion by Novartis, scFv fusion by Immunomedics, scFv fusion by Changzhou Adam Biotech Inc (CN 102250246), TvAb by Roche (WO 2012025525, WO 2012025530), mAb² by f-Star (WO2008/003116), and dual scFv-fusions. It also should be understood that the term antibody, unless specified otherwise, also includes polyclonal antibodies, monoclonal antibodies (such as human monoclonal antibodies), antibody mixtures (recombinant polyclonals) for instance generated by technologies exploited by Symphogen and Merus (Oligoclonics), multimeric Fc proteins as described in WO2015/158867, fusion proteins as described in WO2014/031646 and antibody-like polypeptides, such as chimeric antibodies and humanized antibodies. An antibody as generated can potentially possess any isotype.

The term “full-length antibody” when used herein, refers to an antibody which contains all heavy and light chain constant and variable domains corresponding to those that are normally found in a wild-type antibody of that isotype.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations, insertions or deletions introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “chimeric antibody”, as used herein, refers to an antibody in which both chain types are chimeric as a result of antibody engineering. A chimeric chain is a chain that contains a foreign variable domain (originating from a non-human species, or synthetic or engineered from any species including human) linked to a constant region of human origin. The variable domain of a chimeric chain has a V region amino acid sequence which, analyzed as a whole, is closer to non-human species than to human.

The term “humanized antibody”, as used herein, refers to an antibody in which both chain types are humanized as a result of antibody engineering. A humanized chain is typically a chain in which the complementarity determining regions (CDR) of the variable domains are foreign (originating from one species other than human, or synthetic) whereas the remainder of the chain is of human origin. Humanization assessment is based on the resulting amino acid sequence, and not on the methodology per se, which allows protocols other than grafting to be used. The variable domain of a humanized chain has a V region amino acid sequence which, analyzed as a whole, is closer to human than to other species. The terms “monoclonal antibody”, “monoclonal Ab”, “monoclonal antibody composition”, “mAb”, or the like, as used herein refer to a preparation of Ab molecules of single molecular composition. A monoclonal antibody composition displays a single binding specificity and affinity for a particular epitope. Accordingly, the term “human monoclonal antibody” refers to Abs displaying a single binding specificity which have variable and constant regions derived from human germline immunoglobulin sequences. The human mAbs may be generated by a hybridoma which includes a B cell obtained from a transgenic or trans-chromosomal non-human animal, such as a transgenic mouse, having a genome comprising a human heavy chain transgene repertoire and a light chain transgene repertoire, rearranged to produce a functional human antibody and fused to an immortalized cell.

The term “isotype”, as used herein, refers to the immunoglobulin class (for instance IgG1, IgG2, IgG3, IgG4, IgD, IgA1, IgGA2, IgE, or IgM or any allotypes thereof such as IgG1m(za) and IgG1m(f)) that is encoded by heavy chain constant region genes. Further, each heavy chain isotype can be combined with either a kappa (κ) or lambda (λ) light chain. The term “mixed isotype” used herein refers to Fc region of an immunoglobulin generated by combining structural features of one isotype with the analogous region from another isotype thereby generating a hybrid isotype. A mixed isotype may comprise an Fc region having a sequence comprised of two or more isotypes selected from the following IgG1, IgG2, IgG3, IgG4, IgD, IgA1, IgGA2, IgE, or IgM thereby generating combinations such as e.g. IgG1/IgG3, IgG1/IgG4, IgG2/IgG3, IgG2/IgG4 or IgG1/IgA.

The term “antigen-binding region”, “antigen binding region”, “binding region” or antigen binding domain, as used herein, refers to a region of an antibody which is capable of binding to the antigen. This binding region is typically defined by the VH and VL domains of the antibody which may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). The antigen can be any molecule, such as a polypeptide, e.g. present on a cell, bacterium, or virion.

The term “target”, as used herein, refers to a molecule to which the antigen binding region of the antibody binds. The target includes any antigen towards which the raised antibody is directed. The term “antigen” and “target” may in relation to an antibody be used interchangeably and constitute the same meaning and purpose with respect to any aspect or embodiment of the present invention.

The term “epitope” means a protein determinant capable of specific binding to an antibody variable domain. Epitopes usually consist of surface groupings of molecules such as amino acids, sugar side chains or a combination thereof and usually have specific three-dimensional structural characteristics, as well as specific charge characteristics. Conformational and non-conformational epitopes are distinguished in that the binding to the former but not the latter is lost in the presence of denaturing solvents. The epitope may comprise amino acid residues directly involved in the binding (also called immunodominant component of the epitope) and other amino acid residues, which are not directly involved in the binding.

The term “antibody variant” or “variant of a parent antibody” of the present invention is an antibody molecule which comprises one or more mutations as compared to a “parent antibody”. The different terms may be used interchangeably and constitute the same meaning and purpose with respect to any aspect or embodiment of the present invention. Similarly, “a variant of a polypeptide comprising an Fc-region of an immunoglobulin and a binding region” or “a variant of a parent polypeptide comprising an Fc-region of an immunoglobulin and a binding region” of the present invention is a “polypeptide comprising an Fc-region of an immunoglobulin and a binding region”, which comprises one or more mutations as compared to a “parent polypeptide comprising an Fc-region of an immunoglobulin and a binding region”. The different terms may be used interchangeably and constitute the same meaning and purpose with respect to any aspect or embodiment of the present invention. Exemplary mutations include amino acid deletions, insertions, and substitutions of amino acids in the parent amino acid sequence. Amino acid substitutions may exchange a native amino acid for another naturally-occurring amino acid, or for a non-naturally-occurring amino acid derivative. The amino acid substitution may be conservative or non-conservative. In the context of the present invention, conservative substitutions may be defined by substitutions within the classes of amino acids reflected in one or more of the following three tables:

### Amino Acid Residue Classes for Conservative Substitutions

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

### Alternative Conservative Amino Acid Residue Substitution Classes

| | | | |
|---|---|---|---|
| 1 | A | S | T |
| 2 | D | E | |
| 3 | N | Q | |
| 4 | R | K | |
| 5 | I | L | M |
| 6 | F | Y | W |

### Alternative Physical and Functional Classifications of Amino Acid Residues

| | |
|---|---|
| Alcohol group-containing | S and T |
| residues | |
| Aliphatic residues | I, L, V, and M |
| Cycloalkenyl-associated | F, H, W, and Y |
| residues | |
| Hydrophobic residues | A, C, F, G, H, I, L, M, R, T, V, W, and Y |
| Negatively charged | D and E |
| residues | |
| Polar residues | C, D, E, H, K, N, Q, R, S, and T |
| Positively charged | H, K, and R |
| residues | |
| Small residues | A, C, D, G, N, P, S, T, and V |
| Very small residues | A, G, and S |
| Residues involved in | A, C, D, E, G, H, K, N, Q, R, S, |
| turn formation | P, and T |
| Flexible residues | Q, T, K, S, G, N, D, E, and R |

In the context of the present invention, a substitution in a variant is indicated as:

Original amino acid—position—substituted amino acid;

The three letter code, or one letter code, are used, including the codes Xaa and X to indicate amino acid residue. Accordingly, the notation “E345R” or “Glu345Arg” means, that the variant comprises a substitution of Glutamic acid with Arginine in the variant amino acid position corresponding to the amino acid in position 345 in the parent antibody.

Where a position as such is not present in an antibody, but the variant comprises an insertion of an amino acid, for example:

Position—substituted amino acid; the notation, e.g., “448E” is used.

Such notation is particular relevant in connection with modification(s) in a series of homologous polypeptides or antibodies.

Similarly when the identity of the substitution amino acid residues(s) is immaterial:

Original amino acid—position; or “E345”.

For a modification where the original amino acid(s) and/or substituted amino acid(s) may comprise more than one, but not all amino acid(s), the substitution of Glutamic acid for Arginine, Lysine or Tryptophan in position 345:

“Glu345Arg, Lys, Trp” or “E345R,K,W” or “E345R/K/W” or “E345 to R, K or W” may be used interchangeably in the context of the invention.

Furthermore, the term “a substitution” embraces a substitution into any one of the other nineteen natural amino acids, or into other amino acids, such as non-natural amino acids. For example, a substitution of amino acid E in position 345 includes each of the following substitutions: 345A, 345C, 345D, 345G, 345H, 345F, 345I, 345K, 345L, 345M, 345N, 345P, 345Q, 345R, 345S, 345T, 345V, 345W, and 345Y. This is equivalent to the designation 345X, wherein the X designates any amino acid. These substitutions can also be designated E345A, E345C, etc, or E345A, C, etc, or E345A/C/etc. The same applies to analogy to each and every position mentioned herein, to specifically include herein any one of such substitutions.

As used herein, the term “effector cell” refers to an immune cell which is involved in the effector phase of an immune response, as opposed to the recognition and activation phases of an immune response. Exemplary immune cells include a cell of a myeloid or lymphoid origin, for instance lymphocytes (such as B cells and T cells including cytolytic T cells (CTLs)), killer cells, natural killer cells, macrophages, monocytes, eosinophils, polymorphonuclear cells, such as neutrophils, granulocytes, mast cells, and basophils. Some effector cells express Fc receptors (FcRs) or complement receptors and carry out specific immune functions. In some embodiments, an effector cell such as, e.g., a natural killer cell, is capable of inducing ADCC. For example, monocytes, macrophages, neutrophils, dendritic cells and Kupffer cells which express FcRs, are involved in specific killing of target cells and presenting antigens to other components of the immune system, or binding to cells that present antigens. In some embodiments the ADCC can be further enhanced by antibody driven classical complement activation resulting in the deposition of activated C3 fragments on the target cell. C3 cleavage products are ligands to complement receptors (CRs), such as CR3, expressed on myeloid cells. The recognition of complement fragments by CRs on effector cells may promote enhanced Fc receptor-mediated ADCC. In some embodiments antibody driven classical complement activation leads to C3 fragments on the target cell. These C3 cleavage products may promote direct complement-dependent cellular cytotoxicity (CDCC). In some embodiments, an effector cell may phagocytose a target antigen, target particle or target cell. The expression of a particular FcR or complement receptor on an effector cell may be regulated by humoral factors such as cytokines. For example, expression of FcγRI has been found to be up-regulated by interferon γ (IFN γ) and/or G-CSF. This enhanced expression increases the cytotoxic activity of FcγRI-bearing cells against targets. An effector cell can phagocytose a target antigen or phagocytose or lyse a target cell. In some embodiments antibody driven classical complement activation leads to C3 fragments on the target cell. These C3 cleavage products may promote direct phagocytosis by effector cells or indirectly by enhancing antibody mediated phagocytosis.

The term “Fc effector functions,” as used herein, is intended to refer to functions that are a consequence of binding a polypeptide or antibody to its target, such as an antigen, on a cell membrane wherein the Fc effector function is attributable to the Fc region of the polypeptide or antibody. Examples of Fc effector functions include (i) C1q-binding, (ii) complement activation, (iii) complement-dependent cytotoxicity (CDC), (iv) antibody-dependent cell-mediated cytotoxity (ADCC), (v) Fc-gamma receptor-binding, (vi) antibody-dependent cellular phagocytosis (ADCP), (vii) complement-dependent cellular cytotoxicity (CDCC), (viii) complement-enhanced cytotoxicity, (ix) binding to complement receptor of an opsonized antibody mediated by the antibody, (x) opsonisation, and (xi) a combination of any of (i) to (x).

The term “decreased Fc effector function(s)”, as used herein, is intended to refer to an Fc effector function that is decreased for a polypeptide or an antibody when directly compared to the Fc effector function of the parent polypeptide or antibody in the same assay.

The term “clustering-dependent functions,” as used herein, is intended to refer to functions that are a consequence of the formation of antigen complexes after oligomerization of polypeptides or antibodies bound to their antigens, optionally on a cell, on a cell membrane, on a virion, or on another particle. Examples of clustering-dependent effector functions include (i) antibody oligomer formation, (ii) antibody oligomer stability, (iii) antigen oligomer formation, (iv) antigen oligomer stability, (v) induction of apoptosis, (vi) proliferation modulation, such as proliferation reduction, inhibition or stimulation, (vii) modulation of signaling, such as protein phosphorylation reduction, inhibition or stimulation, and (viii) a combination of any of (i) to (vii).

The term “vector,” as used herein, is intended to refer to a nucleic acid molecule capable of inducing transcription of a nucleic acid segment ligated into the vector. One type of vector is a “plasmid”, which is in the form of a circular double stranded DNA loop. Another type of vector is a viral vector, wherein the nucleic acid segment may be ligated into the viral genome. Certain vectors are capable of autonomous replication in a host cell into which they are introduced (for instance bacterial vectors having a bacterial origin of replication and episomal mammalian vectors). Other vectors (such as non-episomal mammalian vectors) may be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the present invention is intended to include such other forms of expression vectors, such as viral vectors (such as replication defective retroviruses, adenoviruses and adeno-associated viruses), which serve equivalent functions.

The term “recombinant host cell” (or simply “host cell”), as used herein, is intended to refer to a cell into which an expression vector has been introduced. It should be understood that such terms are intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. Recombinant host cells include, for example, transfectomas, such as CHO cells, HEK-293 cells, PER.C6, NSO cells, and lymphocytic cells, and prokaryotic cells such as E. coli and other eukaryotic hosts such as plant cells and fungi.

The term “transfectoma”, as used herein, includes recombinant eukaryotic host cells expressing the Ab or a target antigen, such as CHO cells, PER.C6, NSO cells, HEK-293 cells, plant cells, or fungi, including yeast cells.

The term “preparation” refers to preparations of antibody variants and mixtures of different antibody variants which can have an increased ability to form oligomers when interacting with antigen associated with a cell (e.g., an antigen expressed on the surface of the cell), a cell membrane, a virion or other structure, which may result in enhanced signaling and/or activation by the antigen.

As used herein, the term “affinity” is the strength of binding of one molecule, e.g. an antibody, to another, e.g. a target or antigen, at a single site, such as the monovalent binding of an individual antigen binding site of an antibody to an antigen.

As used herein, the term “avidity” refers to the combined strength of multiple binding sites between two structures, such as between multiple antigen binding sites of antibodies simultaneously interacting with a target or e.g. between antibody and C1q. When more than one binding interactions are present, the two structures will only dissociate when all binding sites dissociate, and thus, the dissociation rate will be slower than for the individual binding sites, and thereby providing a greater effective total binding strength (avidity) compared to the strength of binding of the individual binding sites (affinity).

As used herein, the term “oligomer” refers to a molecule that consists of more than one but a limited number of monomer units (e.g. antibodies) in contrast to a polymer that, at least in principle, consists of an unlimited number of monomers. Exemplary oligomers are dimers, trimers, tetramers, pentamers and hexamers. Greek prefixes are often used to designate the number of monomer units in the oligomer, for example a tetramer being composed of four units and a hexamer of six units.

The term “oligomerization”, as used herein, is intended to refer to a process that converts monomers to a finite degree of polymerization. Herein, it is observed that, polypeptides, antibodies and/or other dimeric proteins comprising target-binding regions according to the invention can form oligomers, such as hexamers, via non-covalent association of Fc-regions after target binding, e.g., at a cell surface. The oligomerization of antibodies can be evaluated for example in a cell viability assay using anti-DR5 antibodies containing an Fc-Fc enhancing mutation such as E430G or E345R (as described in Examples 13). Fc-Fc-mediated oligomerization of polypeptides or antibodies occurs after target binding on a (cell) surface through the intermolecular association of Fc-regions between neighboring polypeptides or antibodies and is increased by introduction of a first mutation in an amino acid corresponding to E430, E345 or S440, with the proviso that the mutation in S440 is S440Y or S440W. Thus, the formation of Fc-Fc-mediated oligomerization upon target binding on a (cell) surface may be determined in an assay using the following peptide DCAWHLGELVWCT, which blocks Fc-Fc interactions. The induction of oligomerization can be assessed by comparing the response of the following groups in an assay; group I) an antibody with a wild type Fc-region, group II) an antibody which is identical to the antibody in group I) except that it comprises a first mutation according to the invention e.g. E430G, group III) the DCAWHLGELVWCT peptide in combination with an antibody which is identical to the antibody in Group I) except that it comprises a first mutation according to the invention e.g. E430G, group IV) an antibody which is identical to the antibody in group I) except that it comprises a first mutation according to the invention e.g. E430G and a second mutation according to the invention e.g. P329D. By comparing the response of group I and group II it is possible to assess the response of enhanced oligomerization. By comparing the response of group II and III it is possible to assess the response of blocking enhanced oligomerization. By comparing the response of group II and IV it is possible to assess if enhanced oligomerization has been maintained. Which assay is suitable to use in the assessment of a response dependent on oligomerization depends on which target antigen the antibody binds to, which is clear to the person skilled in the art. Thus, for antibodies which bind to a target antigen which induces programmed cell death (PCD), such as TNFR-SF with an intracellular death domain e.g. DR5, FAS, DR4, and TNFR1, a suitable assay for determining oligomerization may be a viability assay as described in Example 13. A viability assay may be performed on BxPC-3 cells in the presence of antibody according to the assay groups described above, i.e. group I, group II, group III and/or group IV. The BxPC-3 cells are incubated with 5 μg/mL or 10 μg/mL of antibody according to the assay groups described above for 3 days at 37° C. The percentage of viable cells may be determined in a CellTiter-Glo luminescent cell viability assay (Promega, Cat no G7571). For antibodies which bind to co-stimulatory immune receptors, such as TNFR-SF without a death domain e.g. OX40, CD40, CD30, CD27, 4-1BB, RANK, and GITR, a suitable assay for determining oligomerization may be an NFAT reporter bioassay. An NFAT reporter bioassay may be performed using Jurkat NFAT reporter cells stably expressing the target antigen which is clear to the person skilled in the art, such as NFκB-luc2/OX40 Jurkat cells that express a luciferase reporter gene under the control of NFAT response elements and have membrane expression of OX40, in the presence of the assay groups described above, i.e. group I, group II group III, group and/IV. The NFκB-luc2/OX40 Jurkat cells are incubated with 1.5 or 5 μg/mL of antibody according to the assay groups described above for 1 day at 37° C. The luciferase expression induced by activation of OX40 may be determined by measuring luminescence signal.

The term “clustering”, as used herein, is intended to refer to oligomerization of antibodies, polypeptides, antigens or other proteins through non-covalent interactions.

The term “Fc-Fc enhancing”, as used herein, is intended to refer to increasing the binding strength between, or stabilizing the interaction between, the Fc regions of two Fc-region containing antibodies or polypeptides so that the polypeptides form oligomers upon target binding.

The term “C1q binding” as used herein, is intended to refer to the binding of C1q in the context of the binding of C1q to an antibody bound to its antigen. The antibody bound to its antigen is to be understood as happening both in vivo and in vitro in the context described herein. C1q binding can be evaluated for example by using antibody immobilized on artificial surfaces or by using antibody bound to a predetermined antigen on a cellular or virion surface (as described in Examples 3 and 11). The binding of C1q to an antibody oligomer is to be understood herein as a multivalent interaction resulting in high avidity binding. A decrease in C1q binding, for example resulting from the introduction of a second mutation in a polypeptide or antibody, may be measured by comparing the C1q binding of the polypeptide or antibody to the C1q binding of its parent polypeptide or antibody without the second mutation within the same assay, as exemplified in Example 3. In short, cells of a suitable origin expressing the target antigen to which the antigen-binding region of the antibody binds may be used in this assay, such a cell line or cell type will be clear to the skilled person. Thus, for antibodies binding to a target antigen on a cancer cell e.g DR5, cancer cells may be suitable in the present assay e.g. BxPC-3 human pancreatic cancer cells (ATCC CRL-1687). Whereas for antibodies binding to OX-40 which is expressed on T cells, T cells may be suitable in the present assay e.g. Jurkat human T cells (ATCC TIB-152). Decreased C1q binding of antibodies according to the invention may be assessed by incubating the appropriate cells at a concentration of 1×10⁶ mL in polystyrene round-bottom 96-well plates with i) a concentration series (0.0003-100 μg/mL) for an antibody comprising a first and a second mutation according to the invention in the presence of 20% C4-depleted serum; and ii) a concentration series (0.0003-100 μg/mL) for a parent antibody comprising a first mutation, but not a second mutation, in the presence of 20% C4-depleted, serum, wherein the antibodies in i) and ii) are incubated with the appropriate cells for 30 min at 4° C., followed by incubating with a labeled anti-human C1q antibody e.g. FITC-labeled rabbit anti-HuC1q and determination of C1q binding by flow cytometry. Alternatively, decreased C1q binding of antibodies according to the invention may be assessed in a C1q binding enzyme-linked immunosorbent assay (ELISA) by coating 96-well Microlon ELISA plates (Greiner, Cat no 655092) with i) a dilution series (0.001-20 μg/mL) of an antibody comprising a first and a second mutation according to the invention; and ii) a dilution series (0.001-20 μg/mL) of an antibody comprising a first mutation, but not a second mutation, in 100 μL PBS and incubating overnight at 4° C., followed by subsequent incubations, with washings in between, with 200 μL/well 0.5×PBS supplemented with 0.025% Tween 20 and 0.1% gelatin for 1 hour at RT (blocking), 100 μL 3% NHS (Sanquin, Ref. M0008AC) for 1 hour at 37° C., 100 μL rabbit anti-human C1q (DAKO, Cat no A0136, 1/4.000) for 1 hour at RT, and 100 μL swine anti-rabbit IgG-horseradish peroxidase (HRP) (DAKO, Cat no P0399, 1/10.000) as detecting antibody for 1 hour at RT; and finally 100 μL substrate with 1 mg/mL 2,2′-azino-bis (3-ethylbenzothiazoline-6-sulfonic acid) (ABTS; Roche, Cat no 11112 597001) for circa 15 min at RT; and stopping the reaction by the addition of 100 μL 2% oxalic acid and measuring absorbance at 405 nm.

As used herein, the term “complement activation” refers to the activation of the classical complement pathway, which is initiated by a large macromolecular complex called C1 binding to antibody-antigen complexes on a surface. C1 is a complex, which consists of 6 recognition proteins C1q and a hetero-tetramer of serine proteases, C1r2C1s2. C1 is the first protein complex in the early events of the classical complement cascade that involves a series of cleavage reactions that starts with the cleavage of C4 into C4a and C4b and C2 into C2a and C2b. C4b is deposited and forms together with C2a an enzymatic active convertase called C3 convertase, which cleaves complement component C3 into C3b and C3a, which forms a C5 convertase This C5 convertase splits C5 in C5a and C5b and the last component is deposited on the membrane and that in turn triggers the late events of complement activation in which terminal complement components C5b, C6, C7, C8 and C9 assemble into the membrane attack complex (MAC). The complement cascade results in the creation of pores due to which causes cell lysis, also known as complement-dependent cytotoxicity (CDC). Complement activation can be evaluated by using C1q efficacy, CDC kinetics CDC assays (as described in WO2013/004842, WO2014/108198) or by the method Cellular deposition of C3b and C4b described in Beurskens et al Apr. 1, 2012 vol. 188 no. 7 3532-3541.

The term “complement-dependent cytotoxicity” (“CDC”), as used herein, is intended to refer to the process of antibody-mediated complement activation leading to lysis of the antibody bound to its target on a cell or virion as a result of pores in the membrane that are created by MAC assembly. CDC can be evaluated by in vitro assay such as a CDC assay in which normal human serum is used as a complement source, as described in Example 2, 3, 4, and 6 or in a C1q concentration series. A decrease in CDC activity, for example resulting from the introduction of a second mutation in a polypeptide or antibody, may be measured by comparing the CDC activity of the polypeptide or antibody to the CDC activity of its parent polypeptide or antibody without the second mutation within the same assay, as exemplified in Example 3 and 4.

The term “antibody-dependent cell-mediated cytotoxicity” (“ADCC”) as used herein, is intended to refer to a mechanism of killing of antibody-coated target cells or virions by cells expressing Fc receptors that recognize the constant region of the bound antibody. ADCC can be determined using methods such as the ADCC assay described in Example 10 or the Luminescent ADCC Reporter BioAssay described in Example 9. A decrease in ADCC activity, for example resulting from the introduction of a second mutation in a polypeptide or antibody, may be measured by comparing the ADCC activity of the polypeptide or antibody to the ADCC activity of its parent polypeptide or antibody without the second mutation within the same assay, as exemplified in Example 10 and 9.

The term “antibody-dependent cellular phagocytosis” (“ADCP”) as used herein is intended to refer to a mechanism of elimination of antibody-coated target cells or virions by internalization by phagocytes. The internalized antibody-coated target cells or virions are contained in a vesicle called a phagosome, which then fuses with one or more lysosomes to form a phagolysosome. ADCP may be evaluated by using an in vitro cytotoxicity assay with macrophages as effector cells and video microscopy as described by van Bij et al. in Journal of Hepatology Volume 53, Issue 4, October 2010, Pages 677-685.

The term “complement-dependent cellular cytotoxicity” (“CDCC”) as used herein is intended to refer to a mechanism of killing of target cells or virions by cells expressing complement receptors that recognize complement 3 (C3) cleavage products that are covalently bound to the target cells or virions as a result of antibody-mediated complement activation. CDCC may be evaluated in a similar manner as described for ADCC.

The term “plasma half-life” as used herein indicates the time it takes to reduce the concentration of polypeptide in the blood plasma to one half of its initial concentration during elimination (after the distribution phase). For antibodies the distribution phase will typically be 1-3 days during which phase there is about 50% decrease in blood plasma concentration due to redistribution between plasma and tissues. The plasma half-life can be measured by methods well-known in the art.

The term “plasma clearance rate” as used herein is a quantitative measure of the rate at which a polypeptide is removed from the blood upon administration to a living organism. The plasma clearance rate may be calculated as the dose/AUC (mL/day/kg), wherein the AUC value (area under the curve) is determined from a concentration-time curve.

The term “antibody-drug conjugate”, as used herein refers to an antibody or Fc-containing polypeptide having specificity for at least one type of malignant cell, a drug, and a linker coupling the drug to e.g. the antibody. The linker is cleavable or non-cleavable in the presence of the malignant cell; wherein the antibody-drug conjugate kills the malignant cell.

The term “antibody-drug conjugate uptake”, as used herein refers to the process in which antibody-drug conjugates are bound to a target on a cell followed by uptake/engulfment by the cell membrane and thereby are drawn into the cell. Antibody-drug conjugate uptake may be evaluated as “antibody-mediated internalization and cell killing by anti-TF ADC in an in vitro killing assay” as described in WO 2011/157741.

The term “apoptosis”, as used herein refers to the process of programmed cell death (PCD) that may occur in a cell. Biochemical events lead to characteristic cell changes (morphology) and death. These changes include blebbing, cell shrinkage, nuclear fragmentation, chromatin condensation, and chromosomal DNA fragmentation. Binding of an antibody to a certain receptor may induce apoptosis.

The term “programmed cell-death” or “PCD”, as used herein refers to the death of a cell in any form mediated by an intracellular program. Different forms of PCD exist, the various types of PCD have in common that they are executed by active cellular processes that can be intercepted by interfering with intracellular signaling. In a particular embodiment, the occurrence of any form of PCD in a cell or tissue may be determined by staining the cell or tissue with conjugated Annexin V, correlating to phosphatidylserine exposure.

The term “Annexin V”, as used herein, refers to a protein of the annexin group that binds phosphatidylserine (PS) on the cell surface.

Fc-receptor binding may be indirectly measured as described in Example 9. Fc-receptor binding may be directly measured as described in Example 21. A decrease in Fc-receptor binding, for example resulting from the introduction of a second mutation in a test antibody or polypeptide, can be measured by comparing the ADCC activity of the polypeptide or antibody to the ADCC activity of its parent polypeptide or antibody without that additional mutation within the same assay, as exemplified in Example 21.

The term “Fc-gamma receptor”, “Fc-gammaR”, “Fcγ receptor”, “FcγR”, may be used interchangeable herein to describe the class of Fc-gamma receptors. This class of receptors includes several family members, FcγRI (CD64), FcγRIIA (CD32), FcγRIIB (CD32), FcγRIIIA (CD16a), FcγRIIIB (CD16b), which differ in their antibody affinities due to their different molecular structure. FcγRI binds to IgG more strongly than FcγRII or FcγRIII does.

The term “FcRn”, as used herein is intended to refer to neonatal Fc receptor which is an Fc receptor. It was first discovered in rodents as a unique receptor capable of transporting IgG from mother's milk across the epithelium of newborn rodent's gut into the newborn's bloodstream. Further studies revealed a similar receptor in humans. In humans, however, it is found in the placenta to help facilitate transport of mother's IgG to the growing fetus and it has also been shown to play a role in monitoring IgG turnover. FcRn binds IgG at acidic pH of 6.0-6.5 but not at neutral or higher pH. Therefore, FcRn can bind IgG from the intestinal lumen (the inside of the gut) at a slightly acidic pH and ensure efficient unidirectional transport to the basolateral side (inside the body) where the pH is neutral to basic (pH 7.0-7.5). This receptor also plays a role in adult salvage of IgG through its occurrence in the pathway of endocytosis in endothelial cells. FcRn receptors in the acidic endosomes bind to IgG internalized through pinocytosis, recycling it to the cell surface, releasing it at the basic pH of blood, thereby preventing it from undergoing lysosomal degradation. This mechanism may provide an explanation for the greater half-life of IgG in the blood compared to other isotypes.

The term “Protein A”, as used herein is intended to refer to a 56 kDa MSCRAMM surface protein originally found in the cell wall of the bacterium Staphylococcus aureus. It is encoded by the spa gene and its regulation is controlled by DNA topology, cellular osmolarity, and a two-component system called ArIS-ArIR. It has found use in biochemical research because of its ability to bind immunoglobulins. It is composed of five homologous Ig-binding domains that fold into a three-helix bundle. Each domain is able to bind proteins from many of mammalian species, most notably IgGs. It binds the heavy chain Fc region of most immunoglobulins (overlapping the conserved binding site of FcRn receptors) and also interacts with the Fab region of the human VH3 family. Through these interactions in serum, IgG molecules bind the bacteria via their Fc region instead of solely via their Fab regions, by which the bacteria disrupts opsonization, complement activation and phagocytosis.

The term “Protein G”, as used herein is intended to refer to an immunoglobulin-binding protein expressed in group C and G Streptococcal bacteria much like Protein A but with differing specificities. It is a 65-kDa (G148 protein G) and a 58 kDa (C40 protein G) cell surface protein that has found application in purifying antibodies through its binding to the Fc region.

### SPECIFIC EMBODIMENTS OF THE INVENTION

The present invention is based on the finding of a need for polypeptides and antibody therapeutics which has enhanced Fc-Fc interactions when bound to the corresponding antigen on the surface of a target cell and which thus forms oligomers upon binding to the antigen, but which do not have the enhanced Fc effector functions such as CDC and/or ADCC, which is normally found for polypeptides and antibodies which forms oligomers such as hexamers upon binding. Surprisingly, the inventors found that by introducing a second mutation corresponding to the following amino acid positions K322 or P329 in the Fc region of a polypeptide or antibody having a first mutation corresponding to one of the following amino acid positions E430, E345 or S440, the enhanced Fc-Fc interactions could be maintained while the Fc effector functions, such as CDC and/or ADCC were decreased compared to a parent of the same polypeptide or antibody only having the first mutation and without a second mutation. In some embodiments one or more effector functions may even be decreased to a level below what is found for a wild type polypeptide or antibody, i.e. without the first and second mutation, but otherwise identical.

In some embodiments, the introduction of a second mutation reduced the Fc effector functions to a level that is comparable to, or less than, the level found in a wild type polypeptide or antibody. In some embodiments, the introduction of a second mutation reduced the Fc effector functions to a level that is comparable to, or less than, the level found for an identical antibody or polypeptide only having the first mutation, i.e. a parent polypeptide or antibody.

In one aspect, the present invention provides for a polypeptide or an antibody comprising an Fc region of a human IgG and an antigen binding region, wherein the Fc region comprises a CH2 and CH3 domain, said Fc region comprises, a (i) first mutation and a (ii) second mutation corresponding to the following amino acid positions in human IgG1 according to EU numbering:
i. first mutation at E430, E345 or S440, with the proviso that the mutation in S440 is S440Y or S440W; and
ii. second mutation at K322 or P329.

The first mutation according to the invention, which is in one of the following amino acid positions E430, E345 or S440 introduces the effect of enhanced Fc-Fc interactions and oligomerization in the polypeptide or antibody. Further, the enhanced oligomerization occurs when the antigen binding region of the polypeptide or antibody is bound to the corresponding target antigen. The enhanced oligomerization generates oligomers, such as e.g. hexamers. The generation of oligomeric structures, such as hexamers, has the effect of increasing Fc effector functions, such as e.g. CDC and/or ADCC, by increasing C1q binding avidity of the polypeptide or antibody. The second mutation according to the invention which is in one of the following amino acid positions; K322 or P329 introduces the effect of decreased Fc effector functions in the polypeptide or antibody. Such decreased Fc effector functions may for instance be decreased C1q binding or CDC activity. Thus, the second mutation is able to counteract the enhanced Fc effector function introduced by the first mutation and thereby generate a polypeptide or antibody having enhanced Fc-Fc interactions and oligomerization, but not having increased Fc effector functions. That is, the Fc effector function is decreased compared to a polypeptide or antibody having the first mutation but not having the second mutation. In some instances where the wild type polypeptide or antibody has increased Fc effector functions, such as CDC, the introduction of the first and second mutation may increase the level of oligomerization, while decreasing the level of CDC to a level that is less than that found for the wild type polypeptide or antibody. Polypeptides or antibodies according to the present invention are of a particular advantage when Fc effector functions are undesirably e.g. when activating an effector cell.

In one embodiment of the present invention, the Fc region does not comprise a mutation in the amino acid positions corresponding to L234 and L235. That is, in one embodiment of the present invention the Fc region comprises the wild type amino acids L and L in the positons corresponding to L234 and L235 in human IgG1, wherein the positions are according to EU numbering.

In one embodiment of the present invention, the Fc region comprises a first and a second mutation, with the proviso that the Fc region comprises L and L in the positions corresponding to L234 and L235.

In one embodiment of the invention, the first mutation is selected from the group consisting of: E430G, E345K, E430S, E430F, E430T, E345Q, E345R, E345Y, S440W and S440Y. In a preferred embodiment of the invention the first mutation is selected from E430G or E345K. Hereby are embodiments provided that allow for enhanced oligomerization of polypeptides or antibodies upon cell surface antigen binding.

In one embodiment of the invention, the polypeptide comprises at least one mutation which is an Fc-Fc enhancing mutation and at least one mutation which decreases an Fc effector function. That is in one embodiment of the invention the polypeptide comprises at least one i) first mutation at an amino acid position corresponding to E430, E345 or S440, with the proviso that the mutation in S440 is S440Y or S440W, and at least one ii) second mutation at an amino acid position corresponding to K322 or P329.

In one embodiment, the polypeptides or antibodies comprise an Fc region comprising a first heavy chain and a second heavy chain, wherein one of the above mentioned first mutations may be present in the first and/or the second heavy chain. In one embodiment of the invention the polypeptides or antibodies comprise an Fc region comprising a first heavy chain and a second heavy chain, wherein the first mutation is present in both the first and second heavy chain. In a preferred embodiment of the invention the polypeptides or antibodies comprise an Fc region comprising a first heavy chain and a second heavy chain, wherein the first mutation and second mutation is present in both the first and second heavy chain. In one embodiment of the invention the polypeptides or antibodies comprise an Fc region comprising a first heavy chain and a second heavy chain, wherein the first mutation is present in the first and second heavy chain and the second mutation is present in both the first and second heavy chain.

In one embodiment of the invention, the second mutation is selected from the group consisting of: K322E, K322D, K322N, P329H, P329K, P329R, P329D, P329E, P329F, P329G, P329I, P329L, P329M, P329N, P329Q, P329S, P329T, P329V, P329W and P329Y. In one embodiment of the invention the second mutation is K322E. Hereby embodiments are provided that allow for inhibition of one or more Fc effector function(s). In one embodiment the second mutation decreases the Fc effector function increased by the first mutation. In one embodiment the second mutation decreases partially the Fc effector function that is increased by the first mutation. In one embodiment the second mutation in a polypeptide or antibody is able to decrease an Fc effector function to a level that is lower than what is found for a polypeptide or antibody with a first mutation, but without a second mutation, i.e. a parent polypeptide or parent antibody. In one embodiment the second mutation in a polypeptide or antibody is able to decrease an Fc effector function to a level that is, comparable to, or lower, than what is found for a polypeptide or antibody without a first and a second mutation, i.e. a wild type polypeptide or antibody. In one embodiment the polypeptide or antibody comprises an Fc region comprising a first heavy chain and a second heavy chain, wherein one of the above mentioned second mutations is present in the first and/or the second heavy chain.

In one embodiment, the second mutation is selected from the group of K322E, K322D, and K322N, and decreases CDC, CDCC, and/or C1q binding. In one embodiment the second mutation is selected from the group of K322E, K322D, and K322N, and decreases C1q binding. In one embodiment the second mutation is K322E, and decreases CDC, CDCC, and/or C1q binding. In one embodiment the second mutation is K322E and decreases C1q binding.

In one embodiment, the second mutation is selected from the group of P329H, P329K, P329R, P329D, P329E, P329F, P329G, P329I, P329L, P329M, P329N, P329Q, P329S, P329T, P329V, P329W, and P329Y, and decreases ADCC, ADCP, FcγR binding, CDC, CDCC, and/or C1q binding. In one embodiment the second mutation is selected from the group of P329H, P329K, P329R, P329D, P329E, P329F, P329G, P329I, P329L, P329M, P329N, P329Q, P329S, P329T, P329V, P329W, and P329Y, and decreases ADCC, FcγR binding, CDC, and/or C1q binding. In one embodiment the second mutation is selected from the group of P329R, P329K, P329D, P329E, and P329G, and decreases ADCC, ADCP, FcγR binding, CDC, CDCC and/or C1q binding. In one embodiment the second mutation is P329R, and decreases ADCC, ADCP, FcγR binding, CDC, CDCC, and/or C1q binding. In one embodiment the second mutation is P329R, and decreases ADCC, ADCP, FcγR binding, CDC, CDCC, and/or C1q binding. In one embodiment the second mutation is P329R, and decreases ADCC, FcγR binding, CDC, and/or C1q binding. In one embodiment the second mutation is P329K, and decreases ADCC, FcγR binding, CDC, and/or C1q binding. In one embodiment the second mutation is P329D, and decreases ADCC, FcγR binding, CDC, and/or C1q binding. In one embodiment the second mutation is P329E, and decreases ADCC, FcγR binding, CDC, and/or C1q binding. In one embodiment the second mutation is P329G, and decreases ADCC, FcγR binding, CDC, and/or C1q binding.

In one embodiment of the invention, the second mutation is P329A. In one embodiment the second mutation is P329A, which decreases ADCC, but not CDC.

In one embodiment of the invention, the second mutation is at position P329, with the proviso that the second mutation is not P329A.

In one embodiment of the present invention, the second mutation is at amino acid position P329, with the proviso that the second mutation is not P329A or P329G.

In a preferred embodiment of the present invention, the polypeptide or antibody comprises a second mutation that is P329R, with the proviso that the polypeptide or antibody does not comprise a mutation in the positions corresponding to L234 and L235 in human IgG1.

In another embodiment of the invention, the second mutation is selected from the group consisting of: P329H, P329K, P329R, P329D, P329E, P329F, P329G, P329I, P329L, P329M, P329N, P329Q, P329S, P329T, P329V, P329W, and P329Y.

In another embodiment of the invention, the second mutation is selected from the group consisting of: P329A, P329H, P329K, P329R, P329D, P329E, P329F, P329G, P329I, P329L, P329M, P329N, P329Q, P329S, P329T, P329V, P329W, and P329Y.

In another embodiment of the invention, the second mutation is selected from the group of: P329R, P329K and P329D.

In one embodiment of the invention, the first mutation is in the amino acid position corresponding to E430 and the second mutation is selected from one of the groups consisting of:
(i) K322E, K322D and K322N, or;
(ii) P329A, P329H, P329K, P329R, P329D, P329E, P329F, P329G, P329I, P329L, P329M, P329N, P329Q, P329S, P329T, P329V, P329W, and P329Y.

In one embodiment of the invention, the Fc region comprises a first mutation in the amino acid position corresponding to E430 and the second mutation is selected from one of the groups consisting of:
(i) K322E, K322D and K322N, or;
(ii) P329A, P329H, P329K, P329R, P329D, P329E, P329F, P329G, P329I, P329L, P329M, P329N, P329Q, P329S, P329T, P329V, P329W, and P329Y; with the proviso that the Fc region comprises L and L in the positions corresponding to L234 and L235.

In one embodiment of the invention, the first mutation is in the amino acid position corresponding to E430 and the second mutation is selected from one of the groups consisting of:
i. K322E, K322D and K322N, or;
ii. P329A, P329H, P329K, P329R, P329D, P329E, P329F, P329I, P329L, P329M, P329N, P329Q, P329S, P329T, P329V, P329W, and P329Y.

In one embodiment of the invention, the first mutation is in the amino acid position corresponding to E430 and the second mutation is selected from one of the groups consisting of:
(i) K322E, K322D and K322N, or;
(ii) P329H, P329K, P329R, P329D, P329E, P329F, P329I, P329L, P329M, P329N, P329Q, P329S, P329T, P329V, P329W, and P329Y.

In one embodiment of the invention, the first mutation is selected from the group consisting of: E430G, E430S, E430F and E430T; and the second mutation is selected from one of the groups consisting of:
(i) K322E, K322D and K322N, or;
(ii) P329H, P329K, P329R, P329D, P329E, P329F, P329I, P329L, P329M, P329N, P329Q, P329S, P329T, P329V, P329W, and P329Y.

In one embodiment of the invention, the first mutation is E430G and the second mutation is selected from the group consisting of: K322E, P329R, P329K and P329D.

In one embodiment of the invention, the Fc region comprises a first mutation which is E430G and a second mutation which is selected from the group consisting of: K322E, P329R, P329K and P329D, wherein the Fc region comprises amino acids L and L in the positions corresponding to L234 and L235.

In one embodiment of the invention, the first mutation is E430G and the second mutation is K322E. In one embodiment of the invention the first mutation is E430G and the second mutation is K322D. In one embodiment of the invention the first mutation is E430G and the second mutation is K322N. In one embodiment of the invention the first mutation is E430G and the second mutation is P329H. In one embodiment of the invention the first mutation is E430G and the second mutation is P329K. In one embodiment of the invention the first mutation is E430G and the second mutation is P329R. In one embodiment of the invention the first mutation is E430G and the second mutation is P329D. In one embodiment of the invention the first mutation is E430G and the second mutation is P329E. In one embodiment of the invention the first mutation is E430G and the second mutation is P329M. In one embodiment of the invention the first mutation is E430G and the second mutation is P329F. In one embodiment of the invention the first mutation is E430G and the second mutation is P329G. In one embodiment of the invention the first mutation is E430G and the second mutation is P329I. In one embodiment of the invention the first mutation is E430G and the second mutation is P329L. In one embodiment of the invention the first mutation is E430G and the second mutation is P329N. In one embodiment of the invention the first mutation is E430G and the second mutation is P329Q. In one embodiment of the invention the first mutation is E430G and the second mutation is P329S. In one embodiment of the invention the first mutation is E430G and the second mutation is P329T. In one embodiment of the invention the first mutation is E430G and the second mutation is P329V. In one embodiment of the invention the first mutation is E430G and the second mutation is P329W. In one embodiment of the invention the first mutation is E430G and the second mutation is P329Y. In one embodiment of the invention the first mutation is E430G and the second mutation is P329A.

In one embodiment of the invention, the first mutation is in the amino acid position corresponding to E345 and the second mutation is selected from one of the groups consisting of:
(i) K322E, K322D and K322N, or;
(ii) P329A, P329H, P329K, P329R, P329D, P329E, P329F, P329G, P329I, P329L, P329M, P329N, P329Q, P329S, P329T, P329V, P329W, and P329Y.

In one embodiment of the invention, the Fc region comprises a first mutation in the amino acid position corresponding to E345 and the second mutation is selected from one of the groups consisting of:
(i) K322E, K322D and K322N, or;
(ii) P329A, P329H, P329K, P329R, P329D, P329E, P329F, P329G, P329I, P329L, P329M, P329N, P329Q, P329S, P329T, P329V, P329W, and P329Y; with the proviso that the Fc region comprises L and L in the positions corresponding to L234 and L235.

In one embodiment of the invention, the first mutation is in the amino acid position corresponding to E345 and the second mutation is selected from one of the groups consisting of:
i. K322E, K322D and K322N, or;
ii. P329A, P329H, P329K, P329R, P329D, P329E, P329F, P329I, P329L, P329M, P329N, P329Q, P329S, P329T, P329V, P329W, and P329Y.

In one embodiment of the invention, the first mutation is in the amino acid position corresponding to E345 and the second mutation is selected from one of the groups consisting of:
(i) K322E, K322D and K322N, or;
(ii) P329H, P329K, P329R, P329D, P329E, P329F, P329I, P329L, P329M, P329N, P329Q, P329S, P329T, P329V, P329W, and P329Y.

In one embodiment of the invention, the first mutation is selected from the group consisting of: E345K, E345R and E345Y; and the second mutation is selected from one of the groups consisting of:
(i) K322E, K322D and K322N, or;
(ii) P329H, P329K, P329R, P329D, P329E, P329F, P329I, P329L, P329M, P329N, P329Q, P329S, P329T, P329V, P329W, and P329Y.

In one embodiment of the invention, the first mutation is E345K and the second mutation is selected from the group consisting of: K322E, P329R, P329K and P329D.

In one embodiment of the invention, the first mutation is E345K and the second mutation is K322E. In one embodiment of the invention the first mutation is E345K and the second mutation is K322D. In one embodiment of the invention the first mutation is E345K and the second mutation is K322N. In one embodiment of the invention the first mutation is E345K and the second mutation is P329H. In one embodiment of the invention the first mutation is E345K and the second mutation is P329K. In one embodiment of the invention the first mutation is E345K and the second mutation is P329R. In one embodiment of the invention the first mutation is E345K and the second mutation is P329D. In one embodiment of the invention the first mutation is E345K and the second mutation is P329E. In one embodiment of the invention the first mutation is E345K and the second mutation is P329M. In one embodiment of the invention the first mutation is E345K and the second mutation is P329F. In one embodiment of the invention the first mutation is E345K and the second mutation is P329G. In one embodiment of the invention the first mutation is E345K and the second mutation is P329I. In one embodiment of the invention the first mutation is E345K and the second mutation is P329L. In one embodiment of the invention the first mutation is E345K and the second mutation is P329N. In one embodiment of the invention the first mutation is E345K and the second mutation is P329Q. In one embodiment of the invention the first mutation is E345K and the second mutation is P329S. In one embodiment of the invention the first mutation is E345K and the second mutation is P329T. In one embodiment of the invention the first mutation is E345K and the second mutation is P329V. In one embodiment of the invention the first mutation is E345K and the second mutation is P329W. In one embodiment of the invention the first mutation is E345K and the second mutation is P329Y. In one embodiment of the invention the first mutation is E345K and the second mutation is P329A.

In one embodiment of the invention, the first mutation is E430S and the second mutation is selected from the group consisting of: K322E, P329R, P329K and P329D.

In one embodiment of the invention, the first mutation is E430S and the second mutation is K322E. In one embodiment of the invention the first mutation is E430S and the second mutation is K322D. In one embodiment of the invention the first mutation is E430S and the second mutation is K322N. In one embodiment of the invention the first mutation is E430S and the second mutation is P329H. In one embodiment of the invention the first mutation is E430S and the second mutation is P329K. In one embodiment of the invention the first mutation is E430S and the second mutation is P329R. In one embodiment of the invention the first mutation is E430S and the second mutation is P329D. In one embodiment of the invention the first mutation is E430S and the second mutation is P329E. In one embodiment of the invention the first mutation is E430S and the second mutation is P329M. In one embodiment of the invention the first mutation is E430S and the second mutation is P329F. In one embodiment of the invention the first mutation is E430S and the second mutation is P329G. In one embodiment of the invention the first mutation is E430S and the second mutation is P329I. In one embodiment of the invention the first mutation is E430S and the second mutation is P329L. In one embodiment of the invention the first mutation is E430S and the second mutation is P329N. In one embodiment of the invention the first mutation is E430S and the second mutation is P329Q. In one embodiment of the invention the first mutation is E430S and the second mutation is P329S. In one embodiment of the invention the first mutation is E430S and the second mutation is P329T. In one embodiment of the invention the first mutation is E430S and the second mutation is P329V. In one embodiment of the invention the first mutation is E430S and the second mutation is P329W. In one embodiment of the invention the first mutation is E430S and the second mutation is P329Y. In one embodiment of the invention the first mutation is E430S and the second mutation is P329A.

In one embodiment of the invention, the first mutation is E430F and the second mutation is selected from the group consisting of: K322E, P329R, P329K and P329D.

In one embodiment of the invention, the first mutation is E430F and the second mutation is K322E. In one embodiment of the invention the first mutation is E430F and the second mutation is K322D. In one embodiment of the invention the first mutation is E430F and the second mutation is K322N. In one embodiment of the invention the first mutation is E430F and the second mutation is P329H. In one embodiment of the invention the first mutation is E430F and the second mutation is P329K. In one embodiment of the invention the first mutation is E430F and the second mutation is P329R. In one embodiment of the invention the first mutation is E430F and the second mutation is P329D. In one embodiment of the invention the first mutation is E430F and the second mutation is P329E. In one embodiment of the invention the first mutation is E430F and the second mutation is P329M. In one embodiment of the invention the first mutation is E430F and the second mutation is P329F. In one embodiment of the invention the first mutation is E430F and the second mutation is P329G. In one embodiment of the invention the first mutation is E430F and the second mutation is P329I. In one embodiment of the invention the first mutation is E430F and the second mutation is P329L. In one embodiment of the invention the first mutation is E430F and the second mutation is P329N. In one embodiment of the invention the first mutation is E430F and the second mutation is P329Q. In one embodiment of the invention the first mutation is E430F and the second mutation is P329S. In one embodiment of the invention the first mutation is E430F and the second mutation is P329T. In one embodiment of the invention the first mutation is E430F and the second mutation is P329V. In one embodiment of the invention the first mutation is E430F and the second mutation is P329W. In one embodiment of the invention the first mutation is E430F and the second mutation is P329Y. In one embodiment of the invention the first mutation is E430F and the second mutation is P329A.

In one embodiment of the invention, the first mutation is E430T and the second mutation is selected from the group consisting of: K322E, P329R, P329K and P329D.

In one embodiment of the invention, the first mutation is E430T and the second mutation is K322E. In one embodiment of the invention the first mutation is E430T and the second mutation is K322D. In one embodiment of the invention the first mutation is E430T and the second mutation is K322N. In one embodiment of the invention the first mutation is E430T and the second mutation is P329H. In one embodiment of the invention the first mutation is E430T and the second mutation is P329K. In one embodiment of the invention the first mutation is E430T and the second mutation is P329R. In one embodiment of the invention the first mutation is E430T and the second mutation is P329D. In one embodiment of the invention the first mutation is E430T and the second mutation is P329E. In one embodiment of the invention the first mutation is E430T and the second mutation is P329M. In one embodiment of the invention the first mutation is E430T and the second mutation is P329F. In one embodiment of the invention the first mutation is E430T and the second mutation is P329G. In one embodiment of the invention the first mutation is E430T and the second mutation is P329I. In one embodiment of the invention the first mutation is E430T and the second mutation is P329L. In one embodiment of the invention the first mutation is E430T and the second mutation is P329N. In one embodiment of the invention the first mutation is E430T and the second mutation is P329Q. In one embodiment of the invention the first mutation is E430T and the second mutation is P329S. In one embodiment of the invention the first mutation is E430T and the second mutation is P329T. In one embodiment of the invention the first mutation is E430T and the second mutation is P329V. In one embodiment of the invention the first mutation is E430T and the second mutation is P329W. In one embodiment of the invention the first mutation is E430T and the second mutation is P329Y. In one embodiment of the invention the first mutation is E430T and the second mutation is P329A.

In one embodiment of the invention, the first mutation is E345Q and the second mutation is selected from the group consisting of: K322E, P329R, P329K and P329D.

In one embodiment of the invention, the first mutation is E345Q and the second mutation is K322E. In one embodiment of the invention the first mutation is E345Q and the second mutation is K322D. In one embodiment of the invention the first mutation is E345Q and the second mutation is K322N. In one embodiment of the invention the first mutation is E345Q and the second mutation is P329H. In one embodiment of the invention the first mutation is E345Q and the second mutation is P329K. In one embodiment of the invention the first mutation is E345Q and the second mutation is P329R. In one embodiment of the invention the first mutation is E345Q and the second mutation is P329D. In one embodiment of the invention the first mutation is E345Q and the second mutation is P329E. In one embodiment of the invention the first mutation is E345Q and the second mutation is P329M. In one embodiment of the invention the first mutation is E345Q and the second mutation is P329F. In one embodiment of the invention the first mutation is E345Q and the second mutation is P329G. In one embodiment of the invention the first mutation is E345Q and the second mutation is P329I. In one embodiment of the invention the first mutation is E345Q and the second mutation is P329L. In one embodiment of the invention the first mutation is E345Q and the second mutation is P329N. In one embodiment of the invention the first mutation is E345Q and the second mutation is P329Q. In one embodiment of the invention the first mutation is E345Q and the second mutation is P329S. In one embodiment of the invention the first mutation is E345Q and the second mutation is P329T. In one embodiment of the invention the first mutation is E345Q and the second mutation is P329V. In one embodiment of the invention the first mutation is E345Q and the second mutation is P329W. In one embodiment of the invention the first mutation is E345Q and the second mutation is P329Y. In one embodiment of the invention the first mutation is E345Q and the second mutation is P329A.

In one embodiment of the invention, the first mutation is E345R and the second mutation is selected from the group consisting of: K322E, P329R, P329K and P329D.

In one embodiment of the invention, the first mutation is E345R and the second mutation is K322E. In one embodiment of the invention the first mutation is E345R and the second mutation is K322D. In one embodiment of the invention the first mutation is E345R and the second mutation is K322N. In one embodiment of the invention the first mutation is E345R and the second mutation is P329H. In one embodiment of the invention the first mutation is E345R and the second mutation is P329K. In one embodiment of the invention the first mutation is E345R and the second mutation is P329R. In one embodiment of the invention the first mutation is E345R and the second mutation is P329D. In one embodiment of the invention the first mutation is E345R and the second mutation is P329E. In one embodiment of the invention the first mutation is E345R and the second mutation is P329M. In one embodiment of the invention the first mutation is E345R and the second mutation is P329F. In one embodiment of the invention the first mutation is E345R and the second mutation is P329G. In one embodiment of the invention the first mutation is E345R and the second mutation is P329I. In one embodiment of the invention the first mutation is E345R and the second mutation is P329L. In one embodiment of the invention the first mutation is E345R and the second mutation is P329N. In one embodiment of the invention the first mutation is E345R and the second mutation is P329Q. In one embodiment of the invention the first mutation is E345R and the second mutation is P329S. In one embodiment of the invention the first mutation is E345R and the second mutation is P329T. In one embodiment of the invention the first mutation is E345R and the second mutation is P329V. In one embodiment of the invention the first mutation is E345R and the second mutation is P329W. In one embodiment of the invention the first mutation is E345R and the second mutation is P329Y. In one embodiment of the invention the first mutation is E345R and the second mutation is P329A.

In one embodiment of the invention, the first mutation is E345Y and the second mutation is selected from the group consisting of: K322E, P329R, P329K and P329D.

In one embodiment of the invention, the first mutation is E345Y and the second mutation is K322E. In one embodiment of the invention the first mutation is E345Y and the second mutation is K322D. In one embodiment of the invention the first mutation is E345Y and the second mutation is K322N. In one embodiment of the invention the first mutation is E345Y and the second mutation is P329H. In one embodiment of the invention the first mutation is E345Y and the second mutation is P329K. In one embodiment of the invention the first mutation is E345Y and the second mutation is P329R. In one embodiment of the invention the first mutation is E345Y and the second mutation is P329D. In one embodiment of the invention the first mutation is E345Y and the second mutation is P329E. In one embodiment of the invention the first mutation is E345Y and the second mutation is P329M. In one embodiment of the invention the first mutation is E345Y and the second mutation is P329F. In one embodiment of the invention the first mutation is E345Y and the second mutation is P329G. In one embodiment of the invention the first mutation is E345Y and the second mutation is P329I. In one embodiment of the invention the first mutation is E345Y and the second mutation is P329L. In one embodiment of the invention the first mutation is E345Y and the second mutation is P329N. In one embodiment of the invention the first mutation is E345Y and the second mutation is P329Q. In one embodiment of the invention the first mutation is E345Y and the second mutation is P329S. In one embodiment of the invention the first mutation is E345Y and the second mutation is P329T. In one embodiment of the invention the first mutation is E345Y and the second mutation is P329V. In one embodiment of the invention the first mutation is E345Y and the second mutation is P329W. In one embodiment of the invention the first mutation is E345Y and the second mutation is P329Y. In one embodiment of the invention the first mutation is E345Y and the second mutation is P329A.

In one embodiment of the invention, the first mutation is selected from the group consisting of: S440Y and S440W, and the second mutation is selected from one of the groups consisting of:
(i) K322E, K322D and K322N, or;
(ii) P329A, P329H, P329K, P329R, P329D, P329E, P329F, P329G, P329I, P329L, P329M, P329N, P329Q, P329S, P329T, P329V, P329W, and P329Y.

In one embodiment of the invention, the first mutation is selected from the group consisting of: S440Y and S440W, and the second mutation is selected from one of the groups consisting of:
(i) K322E, K322D and K322N, or;
(ii) P329A, P329H, P329K, P329R, P329D, P329E, P329F, P329G, P329I, P329L, P329M, P329N, P329Q, P329S, P329T, P329V, P329W, and P329Y;
with the proviso that the Fc region comprises L and L in the positions corresponding to 234 and 235.

In one embodiment of the invention, the first mutation is selected from the group consisting of: S440Y and S440W, and the second mutation is selected from one of the groups consisting of:
i. K322E, K322D and K322N, or;
ii. P329A, P329H, P329K, P329R, P329D, P329E, P329F, P329I, P329L, P329M, P329N, P329Q, P329S, P329T, P329V, P329W, and P329Y.

In one embodiment of the invention, the first mutation is selected from the group consisting of: S440Y and S440W and the second mutation is selected from one of the groups consisting of:
(i) K322E, K322D and K322N, or;
(ii) P329H, P329K, P329R, P329D, P329E, P329F, P329I, P329L, P329M, P329N, P329Q, P329S, P329T, P329V, P329W, and P329Y.

In one embodiment of the invention, the first mutation is S440W and the second mutation is selected from the group consisting of: K322E, P329R, P329K and P329D.

In one embodiment of the invention, the first mutation is S440W and the second mutation is K322E. In one embodiment of the invention the first mutation is S440W and the second mutation is K322D. In one embodiment of the invention the first mutation is S440W and the second mutation is K322N. In one embodiment of the invention the first mutation is S440W and the second mutation is P329H. In one embodiment of the invention the first mutation is S440W and the second mutation is P329K. In one embodiment of the invention the first mutation is S440W and the second mutation is P329R. In one embodiment of the invention the first mutation is S440W and the second mutation is P329D. In one embodiment of the invention the first mutation is S440W and the second mutation is P329E. In one embodiment of the invention the first mutation is S440W and the second mutation is P329M. In one embodiment of the invention the first mutation is S440W and the second mutation is P329F. In one embodiment of the invention the first mutation is S440W and the second mutation is P329G. In one embodiment of the invention the first mutation is S440W and the second mutation is P329I. In one embodiment of the invention the first mutation is S440W and the second mutation is P329L. In one embodiment of the invention the first mutation is S440W and the second mutation is P329N. In one embodiment of the invention the first mutation is S440W and the second mutation is P329Q. In one embodiment of the invention the first mutation is S440W and the second mutation is P329S. In one embodiment of the invention the first mutation is S440W and the second mutation is P329T. In one embodiment of the invention the first mutation is S440W and the second mutation is P329V. In one embodiment of the invention the first mutation is S440W and the second mutation is P329W. In one embodiment of the invention the first mutation is S440W and the second mutation is P329Y. In one embodiment of the invention the first mutation is S440W and the second mutation is P329A.

In one embodiment of the invention, the first mutation is S440Y and the second mutation is selected from the group consisting of: K322E, P329R, P329K and P329D.

In one embodiment of the invention, the first mutation is S440Y and the second mutation is K322E. In one embodiment of the invention the first mutation is S440Y and the second mutation is K322D. In one embodiment of the invention the first mutation is S440Y and the second mutation is K322N. In one embodiment of the invention the first mutation is S440Y and the second mutation is P329H. In one embodiment of the invention the first mutation is S440Y and the second mutation is P329K. In one embodiment of the invention the first mutation is S440Y and the second mutation is P329R. In one embodiment of the invention the first mutation is S440Y and the second mutation is P329D. In one embodiment of the invention the first mutation is S440Y and the second mutation is P329E. In one embodiment of the invention the first mutation is S440Y and the second mutation is P329M. In one embodiment of the invention the first mutation is S440Y and the second mutation is P329F. In one embodiment of the invention the first mutation is S440Y and the second mutation is P329G. In one embodiment of the invention the first mutation is S440Y and the second mutation is P329I. In one embodiment of the invention the first mutation is S440Y and the second mutation is P329L. In one embodiment of the invention the first mutation is S440Y and the second mutation is P329N. In one embodiment of the invention the first mutation is S440Y and the second mutation is P329Q. In one embodiment of the invention the first mutation is S440Y and the second mutation is P329S. In one embodiment of the invention the first mutation is S440Y and the second mutation is P329T. In one embodiment of the invention the first mutation is S440Y and the second mutation is P329V. In one embodiment of the invention the first mutation is S440Y and the second mutation is P329W. In one embodiment of the invention the first mutation is S440Y and the second mutation is P329Y. In one embodiment of the invention the first mutation is S440Y and the second mutation is P329A.

In one embodiment of the invention, the Fc region comprises one or more further mutations. The Fc region comprises a CH2 domain, a CH3 domain and optionally a hinge region. In one embodiment of the invention the Fc region comprises one or more further mutations in the CH2 or CH3 domain. In one embodiment the one or more further mutations are in the CH2 domain. In another embodiment the one or more further mutations are in the CH3 domain.

In one embodiment of the invention, the Fc region comprises:
(i) a first mutation, which is an Fc-Fc enhancing mutation;
(ii) a second mutation, which inhibits Fc effector function(s);
(iii) a further mutation, which prevents oligomerization between Fc regions having the identical further mutation.

In one embodiment of the invention, the Fc region comprises a further mutation in the CH3 domain corresponding to K439 or where the first mutation is not at position S440 the further mutation may be at position S440. In one embodiment of the invention the Fc region comprises a further mutation in the CH3 domain corresponding to one of the following position S440 or K439, with the proviso that the first mutation is not in S440. Polypeptides or antibodies comprising a first and a second mutation according to the present invention and a further mutation at position S440, such as S440K, do not form oligomers with polypeptides or antibodies comprising a further mutation at position S440, such as S440K. Polypeptides or antibodies comprising a first and a second mutation according to the present invention and a further mutation at position K439, such as K439E, do not form oligomers with polypeptides or antibodies comprising a mutation at position K439, such as K439E. In one embodiment of the invention the further mutation is selected from S440K or K439E. Polypeptides or antibodies comprising a further mutation that is K439E or S440K do not form oligomers with polypeptides having the same identical mutation. Without being bound by theory K439E and S440K can be viewed as complementary mutations, thus an Fc region comprising a K439E mutation will not form Fc-Fc interactions with another Fc region comprising a K439E mutation. An Fc region comprising a K439E mutation will however form Fc-Fc interactions with another Fc region comprising a S440K mutation. The same situation is found for Fc regions comprising the S440K mutation, which will not form Fc-Fc interactions with another Fc region comprising the S440K mutation. Thus, a polypeptide or an antibody comprising a K439E mutation will form oligomers with a polypeptide or antibody comprising a S440K mutation in an alternating pattern.

In one embodiment of the invention, the Fc region comprises (i) a first mutation, (ii) a second mutation, (iii) a further mutation, wherein the mutations corresponds to the following amino acid positions in human IgG1, according to EU numbering:
(i) a first mutation E430, E345 or S440, with the proviso that the mutation in S440 is S440Y or S440W;
(ii) a second mutation at E322 or P329;
(iii) a further mutation at K439 or S440, with the proviso that if the further mutation is at S440 then the first mutation is not at S440.

In one embodiment of the invention, the Fc region comprises (i) a first mutation, (ii) a second mutation, (iii) a further mutation, wherein the mutations corresponds to the following amino acid positions in human IgG1, according to EU numbering:
(i) a first mutation E430, E345 or S440, with the proviso that the mutation in S440 is S440Y or S440W;
(ii) a second mutation at E322 or P329;
(iii) a further K439E or S440K mutation, with the proviso that if the further mutation is S440K then the first mutation is not at S440; wherein the Fc region comprises the wild type amino acids L and L in the positions corresponding to L234 and L235.

In one embodiment of the invention, the Fc region comprises a (i) first mutation in the amino acid position corresponding to E430 and (ii) a second mutation, and (iii) a further mutation, wherein the second and further mutation is selected from the following groups consisting of:
(ii) K322E, K322D, K322N, P329A, P329H, P329K, P329R, P329D, P329E, P329F, P329G, P329I, P329L, P329M, P329N, P329Q, P329S, P329T, P329V, P329W, and P329Y;
(iii) K439E and S440K.

In one embodiment of the invention, the Fc region comprises a (i) first mutation in the amino acid position corresponding to E430 and (ii) a second mutation, and (iii) a further mutation, wherein the second and further mutation is selected from the following groups consisting of:
(ii) K322E, K322D, K322N, P329A, P329H, P329K, P329R, P329D, P329E, P329F, P329G, P329I, P329L, P329M, P329N, P329Q, P329S, P329T, P329V, P329W, and P329Y;
(iii) K439E and S440K;
   wherein the Fc region comprises the wild type amino acids L and L in the positions corresponding to L234 and L235.

In one embodiment of the invention, the Fc region comprises (i) a first mutation, and (ii) a second mutation, and (ii) a further mutation, wherein the mutations are selected from the following groups consisting of:
(i) E430G, E430S, E430F and E430T;
(ii) K322E, K322D, K322N, P329A, P329H, P329K, P329R, P329D, P329E, P329F, P329G, P329I, P329L, P329M, P329N, P329Q, P329S, P329T, P329V, P329W, and P329Y;
(iii) K439E and S440K.

In one embodiment of the invention, the Fc region comprises a (i) first mutation in the amino acid position corresponding to E345 and (ii) a second mutation, and (iii) a further mutation, wherein the second and further mutation is selected from the following groups consisting of:
(ii) K322E, K322D, K322N, P329A, P329H, P329K, P329R, P329D, P329E, P329F, P329G, P329I, P329L, P329M, P329N, P329Q, P329S, P329T, P329V, P329W, and P329Y;
(iii) K439E and S440K;
   wherein the Fc region comprises the wild type amino acids L and L in the positions corresponding to L234 and L235.

In one embodiment of the invention, the Fc region comprises (i) a first mutation, and (ii) a second mutation, and (iii) a further mutation, wherein the first, second and further mutation are selected from the following groups consisting of:
(i) E345K, E345R and E345Y;
(ii) K322E, K322D, K322N, P329A, P329H, P329K, P329R, P329D, P329E, P329F, P329G, P329I, P329L, P329M, P329N, P329Q, P329S, P329T, P329V, P329W, and P329Y;
(iii) K439E and S440K.

In one embodiment of the invention, the Fc region comprises (i) a first mutation, and (ii) a second mutation, and (iii) a further mutation, wherein the first, second and further mutation are selected from the following groups consisting of:
(i) S440W and S440Y;
(ii) K322E, K322D, K322N, P329A, P329H, P329K, P329R, P329D, P329E, P329F, P329G, P329I, P329L, P329M, P329N, P329Q, P329S, P329T, P329V, P329W, and P329Y;
(iii) K439E.

In one embodiment of the present invention, the Fc region comprises a further mutation which is a hexamerization-inhibiting mutation corresponding to K439E or S440K in human IgG1, according to EU numbering. That is, in one embodiment of the present invention the Fc region comprises a hexamerization enhancing mutation, such as E430G, and a hexamerization-inhibiting mutation, such as K439E. In one embodiment of the present invention the Fc region comprises a hexamerization enhancing mutation such as E345K and a hexamerization-inhibiting mutation, such as K439E. In another embodiment of the present invention the Fc region comprises a hexamerization enhancing mutation such as E430G and a hexamerization-inhibiting mutation, such as S440K. In one embodiment of the present invention the Fc region comprises a hexamerization enhancing mutation such as E345K and a hexamerization-inhibiting mutation, such as S440K. Hereby are embodiments provided that allow for exclusive hexamerization between combinations of antibodies comprising a K439E mutation and antibodies comprising a S440K mutation.

The polypeptide or antibody according to the invention has at least a first and a second mutation, but as described above may also have additional mutations to introduce additional functions into the polypeptide or antibody. In one embodiment the Fc region comprises at most ten mutations, such as nine mutations, such as eight mutations, such as seven mutations, such as six mutations, such as five mutations, such as four mutations, such as three mutations or such as two mutations.

Hereby, embodiments are provided that allow for polypeptides or antibodies of the invention to have additional mutations which introduces additional features into the polypeptide or antibody. Further, the additional mutations also allow for a variation in the Fc region at positions which are not involved in Fc-Fc interaction, as well as in positions not involved in Fc effector functions. Further, additional mutations may also be due to allelic variations.

In one embodiment of the invention, the polypeptide or antibody has an Fc effector function decreased by at least 20% compared to a parent polypeptide or antibody which is identical to the antibody except that it does not comprise the second mutation. That is the polypeptide or antibody having a first and a second mutation where the second mutation is having the effect of decreasing the effector function of the polypeptide or antibody by at least 20% compared to a parent polypeptide or antibody having only the first mutation. In another embodiment of the invention the polypeptide or antibody has an Fc effector function decreased by at least 30%, at least 40%, at least 50% at least 60%, at least 70% at least 80%, at least 90%, at least 95% compared to a parent polypeptide or antibody having only the first mutation.

In one embodiment of the invention, the polypeptide or antibody does not induce an Fc effector function.

In one embodiment according to the invention, a decrease in Fc effector functions or activity of a polypeptide having a first and second mutation is to be understood as when the polypeptide is compared to a parent polypeptide having the identical antigen binding region and an Fc region having the same first mutation, but lacking the second mutation in the Fc region.

In another embodiment according to the invention, a decrease in Fc effector functions or activity of a polypeptide having a first and second mutation is to be understood as when the polypeptide is compared to a parent polypeptide having the identical antigen binding region and an Fc region and not having the first and second mutation in the Fc region, that is, a wild type antibody.

In one embodiment according to the invention, the second mutation decreases at least one effector function. In one embodiment according to the invention the second mutations decrease more than one effector function. In one embodiment according to the invention the second mutation decreases CDC activity. In one embodiment according to the invention the second mutation decreases ADCC activity. In another embodiment the second mutation decreases CDC and ADCC activity. In one embodiment according to the invention the second mutation decreases FcγRIIIa signaling. In a further embodiment according to the invention the second mutation decrease the CDC activity but not ADCC activity or FcγRIIIa signaling. That is in some embodiments according to the invention the second mutation decreases on or more effector functions, while having no decreasing effect on other effector functions. In one embodiment according to the invention the second mutation decreases CDC activity, but still retained considerable ADCC activity.

In one embodiment of the invention, the Fc effector function is selected from the following group; complement-dependent cytotoxicity (CDC), complement-dependent cell-mediated cytotoxicity, complement activation, antibody-dependent cell-mediated cytotoxicity (ADCC), antibody-dependent cell-mediated phagocytosis, C1q binding and FcγR binding. In one embodiment the Fc effector function is FcγRIIIa signaling. That is the second mutation according to the invention is able to decrease at least one Fc effector function.

Some second mutations show a decrease in more than one effector function. Particular mutations which decrease the CDC activity were also characterized by a decreased ADCC activity and decreased FcγRIIIa binding, such mutations include mutations selected from the group comprising: P329H, P329K, P329R, P329D, P329E, P329F, P329G, P329I, P329L, P329M, P329N, P329Q, P329S, P329T, P329V, P329W, and P329Y. Whereas other mutations were found to have retained CDC activity, while decreasing FcγRIIIa binding and decreasing ADCC activity, such mutations include the ones from the group comprising: P329A. Some second mutations showed no FcγRIa binding such as P329R and P329K. Some second mutations showed some decrease binding to FcγRIa binding such as P329G and P329A.

Hereby, novel polypeptide or antibody-based therapeutics having decreased Fc effector functions is provided. The invention also provides for more selective Fc effector function capabilities of Fc-Fc enhanced polypeptides or antibodies.

In one embodiment of the invention, the polypeptide is an antibody, monospecific antibody, bispecific antibody or multispecific antibody. In one embodiment the polypeptide is a monospecific polypeptide, a bispecific polypeptide or a multispecific polypeptide.

The polypeptide of the invention is not limited to antibodies which have a natural, e.g. a human Fc domain but it may also be an antibody having other mutations than those of the present invention, such as e.g. mutations that affect glycosylation or enables the antibody to be a bispecific antibody. By the term “natural antibody” is meant any antibody which does not comprise any genetically introduced mutations. An antibody which comprises naturally occurring modifications, e.g. different allotypes, is thus to be understood as a “natural antibody” in the sense of the present invention, and can thereby be understood as a parent antibody. Such antibodies may serve as a template for the one or more mutations according to the present invention, and thereby providing the variant antibodies of the invention. An example of a parent antibody comprising other mutations than those of the present invention is the bispecific antibody as described in WO2011/131746 (Genmab), utilizing reducing conditions to promote half-molecule exchange of two antibodies comprising IgG4-like CH3 regions, thus forming bispecific antibodies without concomitant formation of aggregates. Other examples of parent antibodies include but are not limited to bispecific antibodies such as heterodimeric bispecifics: Triomabs (Fresenius); bispecific IgG1 and IgG2 (Rinat neurosciences Corporation); FcAAdp (Regeneron); Knobs-into-holes (Genentech); Electrostatic steering (Amgen, Chugai, Oncomed); SEEDbodies (Merck); Azymetric scaffold (Zymeworks); mAb-Fv (Xencor); and LUZ-Y (Genentech). Other exemplary parent antibody formats include, without limitation, a wild type antibody, a full-length antibody or Fc-containing antibody fragment, a human antibody, humanized antibody, chimeric antibody or any combination thereof.

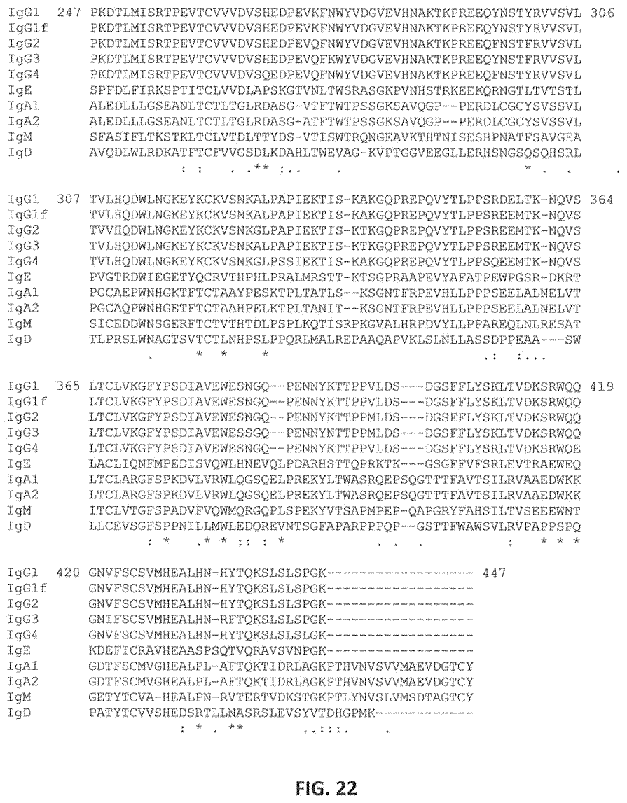

The polypeptide or antibody may be any human antibody of any isotype, e.g. IgG1, IgG2, IgG3, IgG4, IgE, IgD, IgM, or IgA, optionally a human full-length antibody, such as a human full-length IgG1 antibody. In one embodiment of the invention the polypeptide or antibody comprises an Fc region comprising an Fc segment as disclosed in FIG. 22, wherein the Fc segment further comprises a first mutation, a second mutation and/or a further mutation or third mutation as disclosed herein. In one embodiment of the invention the polypeptide or antibody comprises an Fc region comprising an Fc segment in SEQ ID NO: 1, wherein the Fc segment further comprises a first mutation a second mutation and/or a further mutation or a third mutations as disclosed herein. In one embodiment of the invention the polypeptide or antibody comprises an Fc region comprising an Fc segment in SEQ ID NO: 2, wherein the Fc segment further comprises a first mutation a second mutation and/or a further mutation or a third mutations as disclosed herein. In one embodiment of the invention the polypeptide or antibody comprises an Fc region comprising an Fc segment in SEQ ID NO: 3, wherein the Fc segment further comprises a first mutation a second mutation and/or a further mutation or a third mutations as disclosed herein. In one embodiment of the invention the polypeptide or antibody comprises an Fc region comprising an Fc segment in SEQ ID NO: 4, wherein the Fc segment further comprises a first mutation a second mutation and/or a further mutation or a third mutations as disclosed herein. In one embodiment of the invention the polypeptide or antibody comprises an Fc region comprising an Fc segment in SEQ ID NO: 5, wherein the Fc segment further comprises a first mutation a second mutation and/or a further mutation or a third mutations as disclosed herein. In one embodiment of the invention the polypeptide or antibody comprises an Fc region comprising an Fc segment in SEQ ID NO: 6, wherein the Fc segment further comprises a first mutation a second mutation and/or a further mutation or a third mutations as disclosed herein. In one embodiment of the invention the polypeptide or antibody comprises an Fc region comprising an Fc segment in SEQ ID NO: 7, wherein the Fc segment further comprises a first mutation a second mutation and/or a further mutation or a third mutations as disclosed herein. In one embodiment of the invention the polypeptide or antibody comprises an Fc region comprising an Fc segment in SEQ ID NO: 8, wherein the Fc segment further comprises a first mutation a second mutation and/or a further mutation or a third mutations as disclosed herein. In one embodiment of the invention the polypeptide or antibody comprises an Fc region comprising an Fc segment in SEQ ID NO: 9, wherein the Fc segment further comprises a first mutation a second mutation and/or a further mutation or a third mutations as disclosed herein. In one embodiment of the invention the polypeptide or antibody comprises an Fc region comprising an Fc segment in SEQ ID NO: 10, wherein the Fc segment further comprises a first mutation a second mutation and/or a further mutation or a third mutations as disclosed herein.

In one embodiment of the invention, the polypeptide or antibody is a human IgG1 antibody, e.g. the IgG1m(za) or IgG1m(f) allotype.

In one embodiment of the invention, the polypeptide or antibody has an Fc region that is a human IgG1, IgG2, IgG3, IgG4, IgE, IgD, IgM, IgA isotype or a mixed isotype. That is the Fc region of a polypeptide or antibody according to the invention has at least a first and a second mutation introduced into the Fc region corresponding to a human IgG1, IgG2, IgG3, IgG4, IgE, IgD, IgM, IgA isotype or a mixed isotype. In one embodiment of the invention the Fc region is a mixed isotype selected form the following group: IgG1/IgG2, IgG1/IgG3, IgG1/IgG4, IgG2/IgG3, IgG2/IgG4 and IgG3/IgG4. In a mixed isotype the Fc region is comprised of amino acid sequence form more than one isotype.

In one embodiment of the invention, the polypeptide or antibody has an Fc region that is a human IgG1, IgG2, IgG3 or IgG4.

In a preferred embodiment of the invention, the polypeptide or antibody has an Fc region that is a human IgG1 isotype.

In one embodiment of the invention, the polypeptide or antibody has an Fc region that is an IgG1m(f), IgG1m(a), IgG1m(z), IgG1m(x) allotype or mixed allotype.

In one embodiment of the invention, the polypeptide or antibody is a human antibody, humanized antibody or chimeric antibody.

The tumor necrosis factor receptor superfamily (TNFRSF) is a group of cytokine receptors characterized by the ability to bind ligands of the tumor necrosis factor superfamily (TNFSF) via an extracellular cysteine-rich domain. The TNF receptors form trimeric complexes in the plasma membrane. The TNFRSF include the following list of 29 proteins; TNFR1 (Uniprot P19438), FAS (Uniprot P25445), DR3 (Uniprot Q93038), DR4 (Uniprot O00220), DR5 (Uniprot O14763), DR6 (Uniprot O75509), NGFR (Uniprot P08138), EDAR (Uniprot Q9UNE0), DcR1 (Uniprot Q14798), DcR2 (Uniprot Q9UBN6), DcR3 (Uniprot O95407), OPG (Uniprot O00300), TROY (Uniprot Q92956), XEDAR (Uniprot Q9HAV5), LTbR (Uniprot P36941), HVEM (Uniprot Q92956), TWEAKR (Uniprot Q9NP84), CD120b (Uniprot P20333), OX40 (Uniprot P43489), CD40 (Uniprot P25942), CD27 (Uniprot P26842), CD30 (Uniprot P28908), 4-1BB (Uniprot Q07011), RANK (Uniprot Q9Y6Q6), TACI (Uniprot O14836), BLySR (Uniprot Q96RJ3), BCMA (Uniprot Q02223), GITR (Uniprot Q9Y5U5), RELT (Uniprot Q969Z4).

Some TNFRSF are involved in apoptosis and contains an intracellular death domain such as FAS, DR4, DR5, TNFR1, DR6, DR3, EDAR and NGFR. Other TNFRSF are involved in other signal transduction pathways, such as proliferation, survival, and differentiation such as DcR1, DcR2, DcR3, OPG, TROY, XEDAR, LTbR, HVEM, TWEAKR, CD120b, OX40, CD40, CD27, CD30, 4-1BB, RANK, TACI, BLySR, BCMA, GITR, RELT. TNF receptors are expressed in a wide variety of tissues in mammals, especially in leukocytes.

In one embodiment, the antigen binding region binds to a member of the TNFR-SF. In one embodiment the antigen binding region binds to a member of the TNFR-SF which does not comprise an intracellular death domain. In one embodiment the TNFR-SF is selected from the group of: OX40, CD40, CD30, CD27, 4-1BB, RANK, TACI, BLySR, BCMA, RELT and GITR. In one embodiment the TNFR-SF is selected form the group of: FAS, DR4, DR4, TNFR1, DR6, DR3, EDAR, and NGFR.

The polypeptide or antibody according to the invention may bind any target, examples of such targets or antigens according to the invention may be, and is not limited to, directed against are: TNFR1, FAS, DR3, DR4, DR5, DR6, NGFR, EDAR, DcR1, DcR2, DcR3, OPG, TROY, XEDAR, LTbR, HVEM, TWEAKR, CD120b, OX40, CD40, CD27, CD30, 4-1BB, RANK, TACI, BLySR, BCMA, GITR, RELT.

### Multispecific Antibodies

In one aspect, the present invention provides a polypeptide or antibody comprising a first Fc region of a human IgG and a first antigen binding region, a second Fc region of a human IgG and a second antigen binding region, wherein said first and second Fc regions comprises a (i) first mutation and a (ii) second mutation and a (iii) third mutation corresponding to the following positions in human IgG1 according to EU numbering:
(i) first mutation at E430, E345 or S440;
(ii) second mutation at K322 or P329;
(iii) third mutation at F405 or K409;
wherein the third mutation is different from the first Fc region and the second Fc region so that if the first Fc region has a mutation in position F405 then second Fc region has a mutation in K409 and vice versa.

Hereby, embodiments are provided wherein the first Fc region and the second Fc region are not identical due to the (iii) third mutation is not located in the same position in the first and second Fc region.

It is to be understood that any embodiment of the present invention described herein may be used in a multispecific antibody aspect described below.

Thus, in one embodiment the variant of the present invention is an antibody selected from a monospecific antibody, bispecific antibody or multispecific antibody.

In a particular embodiment, the bispecific antibody has the format described in WO 2011/131746.

In another aspect, the invention relates to a polypeptide or antibody which is a bispecific polypeptide or antibody comprising a first antigen-binding region, a second antigen-binding region and an Fc region comprising a first CH2-CH3 heavy chain of an immunoglobulin and a second CH2-CH3 heavy chain of an immunoglobulin, wherein 2 the first and second antigen-binding regions bind different epitopes on the same or on different antigens, and wherein the first and/or second CH2-CH3 heavy chain comprises,
(i) a first mutation selected from the group corresponding to E430G, E430S, E430F, E430T, E345K, E345Q, E345R, E345Y, S440Y, and S440W in the Fc region of a human IgG1 heavy chain,
(ii) (ii) a second mutation selected form the group corresponding E322E, P329R, P329K, P329D, and
   wherein the first CH2-CH3 heavy chain comprises a third mutation in an amino acid residue selected from those corresponding to K409, T366, L368, K370, D399, F405, and Y407 in the Fc region of a human IgG1; and
   the second CH2-CH3 heavy chain comprises a third mutation in an amino acid residue selected from those corresponding to F405, T366, L368, K370, D399, Y407 and K409 in the Fc region of a human IgG1, and wherein the third mutation in the first polypeptide is different from the further mutation in the second polypeptide.

The bispecific antibody of the present invention is not limited to a particular format and it may be any of those described herein.

In one particular embodiment of the present invention, (i) the first CH2-CH3 heavy chain comprises a third mutation in the amino acid residue corresponding to K409, such as K409R; and (ii) the second CH2-CH3 heavy chain comprises a third mutation in the amino acid residue corresponding to F405, such as F405L.

In one embodiment of the present invention, the first and/or second CH2-CH3 heavy chain comprises
(i) a first mutation corresponding to E430G,
(ii) a second mutation selected form the group consisting of E322E, K322D, K322N, P329H, P329K, P329R, P329D, P329E, P329f, P329G, P329I, P329L, P329N, P329Q, P329S, P329T, P329V, P329W, P329Y, and
   wherein the first CH2-CH3 heavy chain comprises a third mutation in an amino acid residue corresponding to K409R; and
   the second CH2-CH3 heavy chain comprises a third mutation in an amino acid residue corresponding to F405L.

In one embodiment of the present invention, the first and/or second CH2-CH3 heavy chain comprises
(iii) a first mutation corresponding to E430G,
(iv) a second mutation corresponding to E322E, and
   wherein the first CH2-CH3 heavy chain comprises a third mutation in an amino acid residue corresponding to K409R; and the second CH2-CH3 heavy chain comprises a third mutation in an amino acid residue corresponding to F405L.

In one embodiment of the present invention, the first and/or second CH2-CH3 heavy chain comprises
(i) a first mutation corresponding to E430G,
(ii) a second mutation corresponding to P329R, and
   wherein the first CH2-CH3 heavy chain comprises a third mutation in an amino acid residue corresponding to K409R; and the second CH2-CH3 heavy chain comprises a third mutation in an amino acid residue corresponding to F405L.

In one embodiment of the present invention, the first and/or second CH2-CH3 heavy chain comprise
(i) a first mutation corresponding to E430G,
(ii) a second mutation corresponding to P329K, and
   wherein the first CH2-CH3 heavy chain comprises a third mutation in an amino acid residue corresponding to K409R; and
   the second CH2-CH3 heavy chain comprises a third mutation in an amino acid residue corresponding to F405L.

In one embodiment of the present invention the first and/or second CH2-CH3 heavy chain comprises
(i) a first mutation corresponding to E430G,
(ii) a second mutation corresponding to P329D, and
   wherein the first CH2-CH3 heavy chain comprises a third mutation in an amino acid residue corresponding to K409R; and
   the second Fc region comprises a third mutation in an amino acid residue corresponding to F405L.

In one embodiment of the present invention, the first and/or second CH2-CH3 heavy chain comprises
(i) a first mutation corresponding to E345K,
(ii) a second mutation selected form the group consisting of E322E, K322D, K322N, P329H, P329K, P329R, P329D, P329E, P329f, P329G, P329I, P329L, P329N, P329Q, P329S, P329T, P329V, P329W, P329Y, and
   wherein the first CH2-CH3 heavy chain comprises a third mutation in an amino acid residue corresponding to K409R; and
   the second CH2-CH3 heavy chain comprises a third mutation in an amino acid residue corresponding to F405L.

In one embodiment of the present invention, the first and/or second CH2-CH3 heavy chain comprises
(iii) a first mutation corresponding to E345K,
(iv) a second mutation corresponding to E322E, and
   wherein the first CH2-CH3 heavy chain comprises a third mutation in an amino acid residue corresponding to K409R; and the second CH2-CH3 heavy chain comprises a third mutation in an amino acid residue corresponding to F405L.

In one embodiment of the present invention, the first and/or second CH2-CH3 heavy chain comprises
(iii) a first mutation corresponding to E345K,
(iv) a second mutation corresponding to P329R, and
   wherein the first CH2-CH3 heavy chain comprises a third mutation in an amino acid residue corresponding to K409R; and
   the second CH2-CH3 heavy chain comprises a third mutation in an amino acid residue corresponding to F405L.

In one embodiment of the present invention the first and/or second CH2-CH3 heavy chain comprises
(iii) a first mutation selected from the group corresponding to E345K,
(iv) a second mutation selected form the group corresponding to P329K, and
   wherein the first CH2-CH3 heavy chain comprises a third mutation in an amino acid residue corresponding to K409R; and
   the second CH2-CH3 heavy chain comprises a third mutation in an amino acid residue corresponding to F405L.

In one embodiment of the present invention, the first and/or second CH2-CH3 heavy chain comprises
(iii) a first mutation selected from the group corresponding to E345K,
(iv) a second mutation selected form the group corresponding to P329D, and
   wherein the first CH2-CH3 heavy chain comprises a third mutation in an amino acid residue corresponding to K409R; and
   the second CH2-CH3 heavy chain comprises a third mutation in an amino acid residue corresponding to F405L.

### Methods of Decreasing Fc Effector Functions of a Polypeptide or Antibody

It is to be understood that the embodiments described below with reference to a polypeptide or antibody refers to a polypeptide or antibody comprising an Fc region having a CH2-CH3 region of an immunoglobulin and an antigen-binding region, a polypeptide or antibody may also be a multispecific polypeptide or antibody having a first CH2-CH3 region of an immunoglobulin and a first antigen-binding region, and a second polypeptide or antibody having a second Fc region comprising a second CH2-CH3 region of an immunoglobulin and a second antigen-binding region.

In one aspect, the present invention relates to a method of decreasing an Fc effector function of a polypeptide or antibody comprising an Fc region of a human immunoglobulin and an antigen binding region, wherein the Fc region comprises a CH2 and CH3 domain, said Fc region comprising a (i) first mutation corresponding to the following positions in human IgG1 according to EU numbering: E430, E345 or S440, which method comprises introducing a (ii) second mutation corresponding to the following positions in human IgG1 according to EU numbering: K322 or P329. The first mutation according to the invention which is in one of the following positions E430, E345 or S440 introduces the effect of enhanced Fc-Fc interactions of the polypeptide or antibody. The second mutation according to the invention which is in one of the following positions K322 or P329 introduces the effect of decreased Fc effector functions in the polypeptide or antibody as also described above.

In one embodiment of the invention, the first mutation is selected from the group consisting of: E430G, E345K, E430S, E430F, E430T, E345Q, E345R, E345Y, S440W and S440Y. Hereby embodiments are provided in which the first mutation enhances Fc-Fc interactions.

In a preferred embodiment of the invention, the first mutation is selected from E430G or E345K.

In one embodiment, the present invention relates to a method of decreasing an Fc effector function or activity of a polypeptide or antibody having a first Fc-Fc enhancing mutation by introducing a second mutation. It is to be understood that the method of decreasing an Fc effector function is determined when the polypeptide or antibody is compared to a parent polypeptide or antibody having the identical antigen binding region and an Fc region having the identical first mutation in the Fc region, but lacking the second mutation in the Fc region. In some embodiments the method for decreasing the Fc effector function or activity reduces the effector functions to a level which is lower or comparable to the level of a parent polypeptide or antibody having the identical antigen binding region and Fc region but not having the first and second mutation in the Fc region.

In one embodiment of the invention, the second mutation is selected from the group consisting of: K322E, K322D, K322N, P329H, P329K, P329R, P329D, P329E, P329F, P329G, P329I, P329L, P329M, P329N, P329Q, P329S, P329T, P329V, P329W and P329Y.

In one embodiment of the invention, the method relates to decreasing an Fc effector function such as CDC, CDCC and/or C1q binding wherein the method comprises introducing a second mutation selected from the following group of K322E, K322D and K322N.

In one embodiment of the invention, the method relates to decreasing an Fc effector function such as ADCC, ADCP, FcγR binding, CDC CDCC and/or C1q binding wherein the method comprises introducing a second mutation selected from the following group of P329H, P329K, P329R, P329D, P329E, P329F, P329G, P329I, P329L, P329M, P329N, P329Q, P329S, P329T, P329V, P329W, and P329Y

In a preferred embodiment of the invention, the second mutation is selected from the group of: K322E, P329R, P329K and P329D.

In one embodiment of the invention, the second mutation is at position P329, with the proviso that the second mutation is not P329A.

In one embodiment, the present invention relates to a method of decreasing an effector function of a polypeptide or antibody wherein the Fc region comprises a first mutation corresponding to E430G, which method comprises introducing a second mutation corresponding to E322E.

In one embodiment, the present invention relates to a method of decreasing an effector function of a polypeptide or antibody wherein the Fc region comprises a first mutation corresponding to E430G, which method comprises introducing a second mutation corresponding to E322D. In one embodiment the present invention relates to a method of decreasing an effector function of a polypeptide or antibody wherein the Fc region comprises a first mutation corresponding to E430G, which method comprises introducing a second mutation corresponding to E322N. In one embodiment the present invention relates to a method of decreasing an effector function of a polypeptide or antibody wherein the Fc region comprises a first mutation corresponding to E430G, which method comprises introducing a second mutation corresponding to P329H.

In one embodiment, the present invention relates to a method of decreasing an effector function of a polypeptide or antibody wherein the Fc region comprises a first mutation corresponding to E430G, which method comprises introducing a second mutation corresponding to P329K. In one embodiment the present invention relates to a method of decreasing an effector function of a polypeptide or antibody wherein the Fc region comprises a first mutation corresponding to E430G, which method comprises introducing a second mutation corresponding to P329R. In one embodiment the present invention relates to a method of decreasing an effector function of a polypeptide or antibody wherein the Fc region comprises a first mutation corresponding to E430G, which method comprises introducing a second mutation corresponding to P329D. In one embodiment the present invention relates to a method of decreasing an effector function of a polypeptide or antibody wherein the Fc region comprises a first mutation corresponding to E430G, which method comprises introducing a second mutation corresponding to P329E. In one embodiment the present invention relates to a method of decreasing an effector function of a polypeptide or antibody wherein the Fc region comprises a first mutation corresponding to E430G, which method comprises introducing a second mutation corresponding to P329M. In one embodiment the present invention relates to a method of decreasing an effector function of a polypeptide or antibody wherein the Fc region comprises a first mutation corresponding to E430G, which method comprises introducing a second mutation corresponding to P329F. In one embodiment the present invention relates to a method of decreasing an effector function of a polypeptide or antibody wherein the Fc region comprises a first mutation corresponding to E430G, which method comprises introducing a second mutation corresponding to P329G. In one embodiment the present invention relates to a method of decreasing an effector function of a polypeptide or antibody wherein the Fc region comprises a first mutation corresponding to E430G, which method comprises introducing a second mutation corresponding to P329I. In one embodiment the present invention relates to a method of decreasing an effector function of a polypeptide or antibody wherein the Fc region comprises a first mutation corresponding to E430G, which method comprises introducing a second mutation corresponding to P329L. In one embodiment the present invention relates to a method of decreasing an effector function of a polypeptide or antibody wherein the Fc region comprises a first mutation corresponding to E430G, which method comprises introducing a second mutation corresponding to P329N. In one embodiment the present invention relates to a method of decreasing an effector function of a polypeptide or antibody wherein the Fc region comprises a first mutation corresponding to E430G, which method comprises introducing a second mutation corresponding to P329Q. In one embodiment the present invention relates to a method of decreasing an effector function of a polypeptide or antibody wherein the Fc region comprises a first mutation corresponding to E430G, which method comprises introducing a second mutation corresponding to P329S. In one embodiment the present invention relates to a method of decreasing an effector function of a polypeptide or antibody wherein the Fc region comprises a first mutation corresponding to E430G, which method comprises introducing a second mutation corresponding to P329T. In one embodiment the present invention relates to a method of decreasing an effector function of a polypeptide or antibody wherein the Fc region comprises a first mutation corresponding to E430G, which method comprises introducing a second mutation corresponding to P329V. In one embodiment the present invention relates to a method of decreasing an effector function of a polypeptide or antibody wherein the Fc region comprises a first mutation corresponding to E430G, which method comprises introducing a second mutation corresponding to P329W. In one embodiment the present invention relates to a method of decreasing an effector function of a polypeptide or antibody wherein the Fc region comprises a first mutation corresponding to E430G, which method comprises introducing a second mutation corresponding to P329Y.

In one embodiment, the present invention relates to a method of decreasing an effector function of a polypeptide or an antibody wherein the Fc region comprises a first mutation corresponding to E430G, which method comprises introducing a second mutation selected from the group consisting of: K322E, P329R, P329K and P329D.

In one embodiment, the present invention relates to a method of decreasing an effector function of a polypeptide or antibody wherein the Fc region comprises a first mutation corresponding to E345K, which method comprises introducing a second mutation corresponding to E322E.

In one embodiment, the present invention relates to a method of decreasing an effector function of a polypeptide or antibody wherein the Fc region comprises a first mutation corresponding to E345K, which method comprises introducing a second mutation corresponding to E322D. In one embodiment the present invention relates to a method of decreasing an effector function of a polypeptide or antibody wherein the Fc region comprises a first mutation corresponding to E345K, which method comprises introducing a second mutation corresponding to E322N. In one embodiment the present invention relates to a method of decreasing an effector function of a polypeptide or antibody wherein the Fc region comprises a first mutation corresponding to E345K, which method comprises introducing a second mutation corresponding to P329H.

In one embodiment, the present invention relates to a method of decreasing an effector function of a polypeptide or antibody wherein the Fc region comprises a first mutation corresponding to E345K, which method comprises introducing a second mutation corresponding to P329K. In one embodiment the present invention relates to a method of decreasing an effector function of a polypeptide or antibody wherein the Fc region comprises a first mutation corresponding to E345K, which method comprises introducing a second mutation corresponding to P329R. In one embodiment the present invention relates to a method of decreasing an effector function of a polypeptide or antibody wherein the Fc region comprises a first mutation corresponding to E345K, which method comprises introducing a second mutation corresponding to P329D. In one embodiment the present invention relates to a method of decreasing an effector function of a polypeptide or antibody wherein the Fc region comprises a first mutation corresponding to E345K, which method comprises introducing a second mutation corresponding to P329E. In one embodiment the present invention relates to a method of decreasing an effector function of a polypeptide or antibody wherein the Fc region comprises a first mutation corresponding to E345K, which method comprises introducing a second mutation corresponding to P329M. In one embodiment the present invention relates to a method of decreasing an effector function of a polypeptide or antibody wherein the Fc region comprises a first mutation corresponding to E345K, which method comprises introducing a second mutation corresponding to P329F. In one embodiment the present invention relates to a method of decreasing an effector function of a polypeptide or antibody wherein the Fc region comprises a first mutation corresponding to E345K, which method comprises introducing a second mutation corresponding to P329G. In one embodiment the present invention relates to a method of decreasing an effector function of a polypeptide or antibody wherein the Fc region comprises a first mutation corresponding to E345K, which method comprises introducing a second mutation corresponding to P329I. In one embodiment the present invention relates to a method of decreasing an effector function of a polypeptide or antibody wherein the Fc region comprises a first mutation corresponding to E345K, which method comprises introducing a second mutation corresponding to P329L. In one embodiment the present invention relates to a method of decreasing an effector function of a polypeptide or antibody wherein the Fc region comprises a first mutation corresponding to E345K, which method comprises introducing a second mutation corresponding to P329N. In one embodiment the present invention relates to a method of decreasing an effector function of a polypeptide or antibody wherein the Fc region comprises a first mutation corresponding to E345K, which method comprises introducing a second mutation corresponding to P329Q. In one embodiment the present invention relates to a method of decreasing an effector function of a polypeptide or antibody wherein the Fc region comprises a first mutation corresponding to E345K, which method comprises introducing a second mutation corresponding to P329S. In one embodiment the present invention relates to a method of decreasing an effector function of a polypeptide or antibody wherein the Fc region comprises a first mutation corresponding to E345K, which method comprises introducing a second mutation corresponding to P329T. In one embodiment the present invention relates to a method of decreasing an effector function of a polypeptide or antibody wherein the Fc region comprises a first mutation corresponding to E345K, which method comprises introducing a second mutation corresponding to P329V. In one embodiment the present invention relates to a method of decreasing an effector function of a polypeptide or antibody wherein the Fc region comprises a first mutation corresponding to E345K, which method comprises introducing a second mutation corresponding to P329W. In one embodiment the present invention relates to a method of decreasing an effector function of a polypeptide or antibody wherein the Fc region comprises a first mutation corresponding to E345K, which method comprises introducing a second mutation corresponding to P329Y.

In one embodiment, the present invention relates to a method of decreasing an effector function of a polypeptide or an antibody wherein the Fc region comprises a first mutation corresponding to E345K, which method comprises introducing a second mutation selected from the group consisting of: K322E, P329R, P329K and P329D.

In one embodiment, the present invention relates to a method wherein the Fc region comprises one or more further mutations in the CH3 domain.

In one embodiment, the present invention relates to a method wherein the Fc region comprises a further mutation in the CH3 domain corresponding to one of the following positions in human IgG1 according to EU numbering: S440 or K439. In one embodiment of the invention the Fc region comprises a further mutation in the CH3 domain corresponding to one of the following position S440 or K439, with the proviso that the further mutation is not in position S440 if the first mutation is in S440. Polypeptides or antibodies comprising a first and a second mutation according to the present invention and a further mutation at position S440 such as S440K do not form oligomers with polypeptides or antibodies comprising a mutation at position S440 such as S440K. Polypeptides or antibodies comprising a first and a second mutation according to the present invention and a further mutation at position K439 such as K439E do not form oligomers with polypeptides or antibodies comprising a mutation at position K439 such as K439E. Hereby a method is provided that allows for the formation of oligomers between polypeptides or antibodies wherein a first polypeptide or antibody comprises a K439E mutation and the second polypeptide or antibody comprises a S440K mutation. In this way oligomers such as e.g. hexamers can be forced to be formed in certain patterns of first and second polypeptides. This may be of interest in methods where the polypeptides bind different targets or epitopes and oligomers should be formed in combinations of these different targets or epitopes.

In one embodiment, the present invention relates to a method wherein the further mutation is selected from S440K or K439E.

In one embodiment, the present invention relates to a method of decreasing an Fc effector function, wherein the Fc effector function is decreased by at least 20% compared to a parent polypeptide or parent antibody which is identical to the polypeptide or with an identical first mutation, but without a second mutation. In another embodiment of the invention the polypeptide or antibody has an Fc effector function decreased by at least 30%, at least 40%, at least 50% at least 60%, at least 70% at least 80%, at least 90%, at least 95% compared to a parent polypeptide or antibody having only the first mutation.

In one embodiment, the present invention relates to a method of decreasing an Fc effector function, wherein the Fc effector function is selected from the following group; complement dependent cytotoxicity (CDC), complement dependent cell-mediated cytotoxicity (CDCC), antibody-dependent cell-mediated cytotoxicity (ADCC), antibody dependent cell-mediated phagocytosis (ADCP), C1q binding and FcγR binding.

In one embodiment, the present invention relates to a method of decreasing ADCC, wherein ADCC is decreased by at least 20%, at least 50%, at least 60%, at least, 70%, at least, 80%, at least, 90%, at least 100% compared to a comparison antibody which is identical to the antibody except that it does not comprise the second mutation.

In one embodiment, the present invention relates to a method of decreasing CDC, wherein CDC is decreased by at least 20%, at least 50%, at least 60%, at least, 70%, at least, 80%, at least, 90%, at least 100% compared to a comparison antibody which is identical to the antibody except that it does not comprise the second mutation.

In one embodiment, the present invention relates to a method of decreasing C1q binding, wherein C1q binding is decreased by at least 20%, at least 50%, at least 60%, at least, 70%, at least, 80%, at least, 90%, at least 100% compared to a comparison antibody which is identical to the antibody except that it does not comprise the second mutation.

In one embodiment, the present invention relates to a method of decreasing Fc-gamma receptor binding, wherein Fc-gamma receptor binding is decreased by at least 20%, at least 50%, at least 60%, at least, 70%, at least, 80%, at least, 90%, at least 100% compared to a comparison antibody which is identical to the antibody except that it does not comprise the second mutation.

In one embodiment, the present invention relates to a method of decreasing Fc-gamma receptor binding, wherein Fc-gamma receptor binding is decreased by at least 20%, at least 50%, at least 60%, at least, 70%, at least, 80%, at least, 90%, at least 100% compared to a comparison antibody which is identical to the antibody except that it does not comprise the first and second mutation.

In a preferred embodiment, the present invention relates to a method of decreasing Fc-gamma receptor I binding, wherein Fc-gamma receptor I binding is decreased by at least 20%, at least 50%, at least 60%, at least, 70%, at least, 80%, at least, 90%, at least 100% compared to a comparison antibody which is identical to the antibody except that it does not comprise the second mutation.

In a preferred embodiment, the present invention relates to a method of decreasing Fc-gamma receptor I binding, wherein Fc-gamma receptor I binding is decreased by at least 20%, at least 50%, at least 60%, at least, 70%, at least, 80%, at least, 90%, at least 100% compared to a comparison antibody which is identical to the antibody except that it does not comprise the first and second mutation.

In a preferred embodiment, the present invention relates to a method of decreasing Fc-gamma receptor I binding, wherein Fc-gamma receptor I binding is decreased by at least, 70%, preferably at least, 80%, more preferably at least, 90% or at least 100% compared to a comparison antibody which is identical to the antibody except that it does not comprise the second mutation.

In a preferred embodiment, the present invention relates to a method of decreasing Fc-gamma receptor I binding, wherein Fc-gamma receptor I binding is decreased by at least, 70%, preferably at least, 80%, more preferably at least, 90% or at least 100% compared to a comparison antibody which is identical to the antibody except that it does not comprise the first and second mutation. Thus, the method comprises decreasing Fc-gamma receptor I binding to a level that is decreased compared to a wild type Fc region.

### Compositions

It is to be understood that the embodiments described below with reference to a polypeptide or antibody refers to a polypeptide or antibody comprising an Fc region having a CH2-CH3 region of an immunoglobulin and an antigen-binding region, a polypeptide or antibody may also be a multispecific polypeptide or antibody comprising a first antigen-binding region, a second antigen-binding region and an Fc region comprising a first CH2-CH3 heavy chain of an immunoglobulin and a second CH2-CH3 heavy chain of an immunoglobulin.

The invention also relates to compositions comprising polypeptides or antibodies described herein and variations hereof. Specific aspects and embodiments will be described below. Furthermore, such polypeptide or antibody may be obtained according to any method described herein.

In one aspect, the present invention relates to a composition comprising at least one polypeptide or antibody described herein.

In one embodiment of the present invention, the composition comprises one or more polypeptides or antibodies according to any aspect or embodiment described herein.

In one embodiment of the present invention, the composition comprises a first polypeptide or antibody and a second polypeptide or antibody as described in any aspect or embodiment herein.

In one embodiment of the invention, the composition comprises a first and a second polypeptide or antibody, wherein the first and the second polypeptide or antibody comprises an Fc region comprising,
(i) a first mutation, which is an Fc-Fc enhancing mutation;
(ii) a second mutation, which inhibits Fc effector function(s);
(iii) a further mutation, which prevents oligomerization between Fc regions having the identical further mutation, wherein the first and the second polypeptide or antibody does not comprise the same further mutation.

In one embodiment of the present invention, the composition comprises a first polypeptide or antibody and a second polypeptide or antibody wherein the first and second polypeptide or antibody comprises a i) first mutation, a ii) second mutation and iii) a further mutation wherein the first and the second polypeptide or antibody does not comprise the same further mutation. Thus, the composition comprises a first polypeptide or antibody comprising a first Fc region and a second polypeptide or antibody comprising a second Fc region.

In one embodiment of the invention, the composition comprises a first polypeptide or antibody comprising a first antigen-binding region and a first Fc region, a second polypeptide or antibody comprising second antigen-binding region and a second Fc region, wherein the first and second Fc region comprises (i) a first mutation, (ii) a second mutation, (iii) a further mutation, wherein the mutations corresponds to the following amino acid positions in human IgG1, according to EU numbering:
(i) a first mutation E430, E345 or S440, with the proviso that the mutation in S440 is S440Y or S440W;
(ii) a second mutation at E322 or P329;
(iii) a further mutation at K439 or S440, with the proviso that if the further mutation is at S440 then the first mutation is not at S440, with the proviso that the first and second Fc region does not comprise a further mutation in the same amino acid position.

In one embodiment of the invention, the composition comprises a first polypeptide or antibody comprising a first antigen-binding region and a first Fc region, a second polypeptide or antibody comprising second antigen-binding region and a second Fc region, wherein the first and second Fc region comprises (i) a first mutation, (ii) a second mutation, (iii) a further mutation, wherein the mutations corresponds to the following amino acid positions in human IgG1, according to EU numbering:
(i) a first mutation E430, E345 or S440, with the proviso that the mutation in S440 is S440Y or S440W;
(ii) a second mutation at E322 or P329;
(iii) a further mutation at K439 in the first Fc region and a further mutation at S440 in the second Fc region, with the proviso that if the further mutation is at S440 then the first mutation is not at S440.

In one embodiment of the invention, the composition comprises a first polypeptide or antibody comprising a first antigen-binding region and a first Fc region, a second polypeptide or antibody comprising second antigen-binding region and a second Fc region, wherein the first and second Fc region comprises (i) a first mutation, (ii) a second mutation, (iii) a further mutation, wherein the mutations corresponds to the following amino acid positions in human IgG1, according to EU numbering:
(i) a first mutation E430, E345 or S440, with the proviso that the mutation in S440 is S440Y or S440W;
(ii) a second mutation at E322 or P329;
(iii) a further mutation at K439 in the second Fc region and a further mutation at S440 in the first Fc region, with the proviso that if the further mutation is at S440 then the first mutation is not at S440.

In one embodiment of the invention, the composition comprises a first polypeptide or antibody comprising a first antigen-binding region and a first Fc region, a second polypeptide or antibody comprising second antigen-binding region and a second Fc region, wherein the first and second Fc region comprises (i) a first mutation, (ii) a second mutation, (iii) a further mutation, wherein the mutations corresponds to the following amino acid positions in human IgG1, according to EU numbering:
(i) a first mutation E430, E345 or S440, with the proviso that the mutation in S440 is S440Y or S440W;
(ii) a second mutation at E322 or P329;
(iii) a further K439E mutation in the first Fc region and a further S440K mutation in the second Fc region.

In one embodiment of the invention, the composition comprises a first polypeptide or antibody comprising a first antigen-binding region and a first Fc region, a second polypeptide or antibody comprising second antigen-binding region and a second Fc region, wherein the first and second Fc region comprises (i) a first mutation, (ii) a second mutation, (iii) a further mutation, wherein the mutations corresponds to the following amino acid positions in human IgG1, according to EU numbering:
(i) a first mutation E430, E345 or S440, with the proviso that the mutation in S440 is S440Y or S440W;
(ii) a second mutation at E322 or P329;
(iii) a further S440K mutation in the first Fc region and a further E439E mutation in the second Fc region.

In one embodiment of the invention, the composition comprises a first polypeptide or antibody comprising a first antigen-binding region and a first Fc-region, a second polypeptide or antibody comprising second antigen-binding region and a second Fc-region, wherein the first and second Fc-region comprises (i) a first mutation, (iii) a further mutation, and the first and/or second Fc region comprises (ii) a second mutation, wherein the mutations corresponds to the following amino acid positions in human IgG1, according to EU numbering:
(i) a first mutation E430, E345 or S440, with the proviso that the mutation in S440 is S440Y or S440W;
(ii) a second mutation at E322 or P329;
(iii) a further K439E mutation in the first Fc region and a further S440K mutation in the second Fc region.

Hereby embodiments are provided wherein either both the first and the second polypeptide or antibody has a decreased Fc effector function, or only the first or the second polypeptide has a decreased Fc effector function.

In one embodiment of the invention, the composition comprises a first polypeptide or antibody comprising a first antigen-binding region and a first Fc region, a second polypeptide or antibody comprising second antigen-binding region and a second Fc region, wherein the first and second Fc region comprises (i) a first mutation in the amino acid position corresponding to E430, and (ii) a second mutation, and (iii) a further mutation, wherein the second and further mutations are selected from the following groups consisting of:
(ii) K322E, K322D, K322N, P329A, P329H, P329K, P329R, P329D, P329E, P329F, P329G, P329I, P329L, P329M, P329N, P329Q, P329S, P329T, P329V, P329W, and P329Y;
(iii) K439E and S440K, wherein the first and the second Fc region do not comprise the same further mutation.

In one embodiment of the invention, the composition comprises a first polypeptide or antibody comprising a first antigen-binding region and a first Fc region, a second polypeptide or antibody comprising second antigen-binding region and a second Fc region, wherein the first and second Fc region comprises (i) a first mutation in the amino acid position corresponding to E345, and (ii) a second mutation, and (iii) a further mutation, wherein the second and further mutations are selected from the following groups consisting of:
(ii) K322E, K322D, K322N, P329A, P329H, P329K, P329R, P329D, P329E, P329F, P329G, P329I, P329L, P329M, P329N, P329Q, P329S, P329T, P329V, P329W, and P329Y;
(iii) K439E and S440K, wherein the first and the second Fc region do not comprise the same further mutation.

In one embodiment of the invention, the composition comprises a first polypeptide or antibody comprising a first antigen-binding region and a first Fc region, a second polypeptide or antibody comprising second antigen-binding region and a second Fc region, wherein the first Fc region comprises (i) a first E430G mutation and (ii) a second mutation, and (iii) a further mutation, wherein the second and further mutations are selected from the following groups consisting of:
(ii) K322E, K322D, K322N, P329A, P329H, P329K, P329R, P329D, P329E, P329F, P329G, P329I, P329L, P329M, P329N, P329Q, P329S, P329T, P329V, P329W, and P329Y;
(iii) K439E and S440K, wherein the first and the second Fc region do not comprise the same further mutation.

In one embodiment of the invention, the composition comprises a first polypeptide or antibody comprising a first antigen-binding region and a first Fc region, a second polypeptide or antibody comprising second antigen-binding region and a second Fc region, wherein the first Fc region comprises (i) a first E430G mutation and (ii) a second mutation, and (iii) a further mutation, wherein the second and further mutations are selected from the following groups consisting of:
(ii) K322E, P329K, P329R, P329D;
(iii) K439E and S440K, wherein the first and the second Fc region do not comprise the same further mutation.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E430G mutation and (ii) a second K322E mutation, and (iii) a further mutation, wherein the further mutations are selected from the group consisting of:
(iii) K439E and S440K, wherein the first and the second Fc region do not comprise the same further mutation.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E430G mutation and (ii) a second P329K mutation, and (iii) a further mutation, wherein the further mutations are selected from the group consisting of:
(iii) K439E and S440K, wherein the first and the second Fc region do not comprise the same further mutation.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E430G mutation and (ii) a second P329R mutation, and (iii) a further mutation, wherein the further mutations are selected from the group consisting of:
(iii) K439E and S440K, wherein the first and the second Fc region do not comprise the same further mutation.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E430G mutation and (ii) a second P329D mutation, and (iii) a further mutation, wherein the further mutations are selected from the group consisting of:
(iii) K439E and S440K, wherein the first and the second Fc region do not comprise the same further mutation.

In one embodiment of the invention, the composition comprises a first polypeptide or antibody comprising a first antigen-binding region and a first Fc region, a second polypeptide or antibody comprising second antigen-binding region and a second Fc region, wherein the first Fc region comprises (i) a first E345K mutation and (ii) a second mutation, and (iii) a further mutation, wherein the second and further mutations are selected from the following groups consisting of:
(ii) K322E, K322D, K322N, P329A, P329H, P329K, P329R, P329D, P329E, P329F, P329G, P329I, P329L, P329M, P329N, P329Q, P329S, P329T, P329V, P329W, and P329Y;
(iii) K439E and S440K, wherein the first and the second Fc region do not comprise the same further mutation.

In one embodiment of the invention, the composition comprises a first polypeptide or antibody comprising a first antigen-binding region and a first Fc region, a second polypeptide or antibody comprising second antigen-binding region and a second Fc region, wherein the first Fc region comprises (i) a first E345K mutation and (ii) a second mutation, and (iii) a further mutation, wherein the second and further mutations are selected from the following groups consisting of:
(ii) K322E, P329K, P329R, P329D;
(iii) K439E and S440K, wherein the first and the second Fc region do not comprise the same further mutation.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345K mutation and (ii) a second K322E mutation, and (iii) a further mutation, wherein the further mutations are selected from the group consisting of:
(iii) K439E and S440K, wherein the first and the second Fc region do not comprise the same further mutation.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345K mutation and (ii) a second P329K mutation, and (iii) a further mutation, wherein the further mutations are selected from the group consisting of:
(iii) K439E and S440K, wherein the first and the second Fc region do not comprise the same further mutation.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345K mutation and (ii) a second P329R mutation, and (iii) a further mutation, wherein the further mutations are selected from the group consisting of:
(iii) K439E and S440K, wherein the first and the second Fc region do not comprise the same further mutation.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345K mutation and (ii) a second P329D mutation, and (iii) a further mutation, wherein the further mutations are selected from the group consisting of:
(iii) K439E and S440K, wherein the first and the second Fc region do not comprise the same further mutation.

In one embodiment of the invention, the composition comprises a first polypeptide or antibody comprising a first antigen-binding region and a first Fc region, a second polypeptide or antibody comprising second antigen-binding region and a second Fc region, wherein the first Fc region comprises (i) a first E345R mutation and (ii) a second mutation, and (iii) a further mutation, wherein the second and further mutations are selected from the following groups consisting of:
(ii) K322E, K322D, K322N, P329A, P329H, P329K, P329R, P329D, P329E, P329F, P329G, P329I, P329L, P329M, P329N, P329Q, P329S, P329T, P329V, P329W, and P329Y;
(iii) K439E and S440K, wherein the first and the second Fc region do not comprise the same further mutation.

In one embodiment of the invention, the composition comprises a first polypeptide or antibody comprising a first antigen-binding region and a first Fc region, a second polypeptide or antibody comprising second antigen-binding region and a second Fc region, wherein the first Fc region comprises (i) a first E345R mutation and (ii) a second mutation, and (iii) a further mutation, wherein the second and further mutations are selected from the following groups consisting of:
(ii) K322E, P329K, P329R, P329D;
(iii) K439E and S440K, wherein the first and the second Fc region do not comprise the same further mutation.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345R mutation and (ii) a second K322E mutation, and (iii) a further mutation, wherein the further mutations are selected from the group consisting of:
(iii) K439E and S440K, wherein the first and the second Fc region do not comprise the same further mutation.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345R mutation and (ii) a second P329K mutation, and (iii) a further mutation, wherein the further mutations are selected from the group consisting of:
(iii) K439E and S440K, wherein the first and the second Fc region do not comprise the same further mutation.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345R mutation and (ii) a second P329R mutation, and (iii) a further mutation, wherein the further mutations are selected from the group consisting of:
(iii) K439E and S440K, wherein the first and the second Fc region do not comprise the same further mutation.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345R mutation and (ii) a second P329D mutation, and (iii) a further mutation, wherein the further mutations are selected from the group consisting of:
(iii) K439E and S440K, wherein the first and the second Fc region do not comprise the same further mutation.

In another embodiment of the invention, the composition comprises a first and a second polypeptide or antibody, wherein the first and the second polypeptide or antibody comprises an Fc region comprising,
(i) a first mutation, which is an Fc-Fc enhancing mutation;
(ii) a further mutation, which prevents oligomerization between Fc regions having the identical further mutation, wherein the first and the second polypeptide or antibody does not comprise the same further mutation,
(iii) and either the first or the second Fc region comprises a second mutation.

Thus, in some embodiments only first or the second polypeptide or antibody comprises a second mutation that decreases Fc effector functions.

In one embodiment of the invention, the composition comprises a first polypeptide or antibody comprising a first antigen-binding region and a first Fc region, a second polypeptide or antibody comprising a second antigen-binding region and a second Fc region, wherein the first and second Fc region comprises (i) a first mutation in an amino acid position selected from the group consisting of: E430, E345 or S440, with the proviso that the mutation in S440 is S440Y or S440W, (ii) a second mutation, (iii) a further mutation E, wherein the mutations corresponds to the following amino acid positions in human IgG1, according to EU numbering:
(iii) a further K439E or S440K mutation, wherein the first and second Fc region does not comprise the same further mutation, and wherein if the first mutation is S440Y or S440W then the further mutation is not S440K;
(ii) and either the first or the second Fc region comprises a second mutation at E322 or P329, but not both.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first mutation in an amino acid position selected from the group consisting of: E430, E345 or S440, with the proviso that the mutation in S440 is S440Y or S440W, and ii) a second mutation in an amino acid positon selected from the group of: E322 and P329, and a iii) further K439E mutation; and the second Fc-region comprises i) a first mutation in an amino acid position selected from the group consisting of: E430 and E345, and a further S440K mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first mutation in an amino acid position selected from the group consisting of: E430 or E345, and ii) a second mutation in an amino acid positon selected from the group of: E322 and P329, and a iii) further S440K mutation; and the second Fc-region comprises i) a first mutation in an amino acid position selected from the group consisting of: E430, E345 or S440, with the proviso that the mutation in S440 is S440Y or S440W, and a further K439E mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E430G and ii) a second E322E mutation, and iii) a further K439E mutation; and the second Fc-region comprises i) a first E430G mutation, and a further S440K mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E430G and ii) a second E322E mutation, and iii) a further S440K mutation; and the second Fc-region comprises i) a first E430G mutation, and a further K322E mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E430G and ii) a second P329R mutation, and iii) a further K439E mutation; and the second Fc-region comprises i) a first E430G mutation, and a further S440K mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E430G and ii) a second P329R mutation, and iii) a further S440K mutation; and the second Fc-region comprises i) a first E430G mutation, and a further K322E mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E430G and ii) a second P329K mutation, and iii) a further K439E mutation; and the second Fc-region comprises i) a first E430G mutation, and a further S440K mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E430G and ii) a second P329K mutation, and iii) a further S440K mutation; and the second Fc-region comprises i) a first E430G mutation, and a further K322E mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E430G and ii) a second P329D mutation, and iii) a further K439E mutation; and the second Fc-region comprises i) a first E430G mutation, and a further S440K mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E430G and ii) a second P329D mutation, and iii) a further S440K mutation; and the second Fc-region comprises i) a first E430G mutation, and a further K322E mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345K and ii) a second E322E mutation, and iii) a further K439E mutation; and the second Fc-region comprises i) a first E345K mutation, and a further S440K mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345K and ii) a second E322E mutation, and iii) a further S440K mutation; and the second Fc-region comprises i) a first E345K mutation, and a further K322E mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345K and ii) a second P329R mutation, and iii) a further K439E mutation; and the second Fc-region comprises i) a first E345K mutation, and a further S440K mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345K and ii) a second P329R mutation, and iii) a further S440K mutation; and the second Fc-region comprises i) a first E345K mutation, and a further K322E mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345K and ii) a second P329K mutation, and iii) a further K439E mutation; and the second Fc-region comprises i) a first E345K mutation, and a further S440K mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345K and ii) a second P329K mutation, and iii) a further S440K mutation; and the second Fc-region comprises i) a first E345K mutation, and a further K322E mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345K and ii) a second P329D mutation, and iii) a further K439E mutation; and the second Fc-region comprises i) a first E345K mutation, and a further S440K mutation. Hereby embodiments are provided where only the first polypeptide or antibody has a decreased Fc effector function.

In one embodiment of the invention, the composition comprises a first polypeptide or antibody comprising a first antigen-binding region and a first Fc-region, a second polypeptide or antibody comprising second antigen-binding region and a second Fc-region, wherein the first Fc-region comprises (i) a first E345K and ii) a second P329D mutation, and iii) a further S440K mutation; and the second Fc-region comprises i) a first E345K mutation, and a further K322E mutation. Hereby embodiments are provided where only the first polypeptide or antibody has a decreased Fc effector function.

In one embodiment of the present invention, the composition comprises a polypeptide or antibody capable of binding to a member of the Tumor Necrosis Factor Receptor Superfamily (TNFR-SF).

In one embodiment of the present invention, the composition comprises a polypeptide or antibody capable of binding to a member of the TNFR-SF with an intracellular death domain selected from the following group consisting of: TNFR1, FAS, DR3, DR4, DR5, DR6, NGFR and EDAR.

In one embodiment of the present invention, the composition comprises a polypeptide or antibody capable of binding to a member of the TNFR-SF without an intracellular death domain selected form the following group consisting of: DcR1, DcR2, DcR3, OPG, TROY, XEDAR, LTbR, HVEM, TWEAKR, CD120b, OX40, CD40, CD27, CD30, 4-1BB, RANK, TACI, BLySR, BCMA, GITR, RELT.

In one embodiment of the present invention, the composition comprises a polypeptide or antibody capable of binding to a member of the TNFR-SF belonging to the group of immune activators consisting of: OX40, CD40, CD27, CD30, 4-1BB, RANK, TACI, BLySR, BCMA, GITR and RELT.

In one embodiment of the present invention, the composition comprises a polypeptide or antibody wherein a first polypeptide and a second polypeptide bind different epitopes on one or more members of the TNFR-SF without an intracellular death domain, selected from the following group consisting of: OX40, CD40, CD27, CD30, 4-1BB, RANK, TACI, BLySR, BCMA, GITR and RELT.

In one embodiment of the present invention, the composition comprises a polypeptide or antibody wherein a first polypeptide binding to one member of the TNFR-SF without an intracellular death domain selected form the following group consisting of: OX40, CD40, CD27, CD30, 4-1BB, RANK, TACI, BLySR, BCMA, GITR and RELT does not block binding of said second antibody binding to one member of the TNFR-SF without an intracellular death domain selected from the following group consisting of: OX40, CD40, CD27, CD30, 4-1BB, RANK, TACI, BLySR, BCMA, GITR and RELT.

In one embodiment of the present invention, the composition comprising a first polypeptide or antibody and a second polypeptide or antibody are present in the composition at a 1:49 to 49:1 molar ratio, such as a 1:1 molar ratio, a 1:2 molar ratio, a 1:3 molar ratio, a 1:4 molar ratio, a 1:5 molar ratio, a 1:6 molar ratio, a 1:7 molar ratio, a 1:8 molar ratio, a 1:9 molar ratio, a 1:10 molar ratio, a 1:15 molar ratio, a 1:20 molar ratio, a 1:25 molar ratio, a 1:30 molar ratio, a 1:35 molar ratio, a 1:40 molar ratio, a 1:45 molar ratio, a 1:50 molar ratio, a 50:1 molar ratio, a 45:1 molar ratio, a 40:1 molar ratio, a 35:1 molar ratio, a 30:1 molar ratio, a 25:1 molar ratio, a 20:1 molar ratio, a 15:1 molar ratio, a 10:1 molar ratio, a 9:1 molar ratio, a 8:1 molar ratio, a 7:1 molar ratio, a 6:1 molar ratio, a 5:1 molar ratio, a 4:1 molar ratio, a 3:1 molar ratio, a 2:1 molar ratio.

In one embodiment of the present invention, the composition comprising a first polypeptide and a second polypeptide and/or any additional polypeptide are present in the composition at an equimolar ratio.

In one embodiment of the present invention, the composition according to any aspect or embodiment is a pharmaceutical composition.

Therapeutic Applications

The polypeptides, antibodies, bispecific antibodies or compositions according to any aspect or embodiment of the present invention may be used as a medicament, i.e. for therapeutic applications.

In one aspect, the present invention provides a polypeptide, antibody or a composition according to any aspect or embodiment disclosed herein for use as a medicament.

In another aspect, the present invention provides a polypeptide, antibody or a composition according to any aspect or embodiment disclosed herein for use in the treatment of cancer, autoimmune disease, inflammatory disease or infectious disease.

In another aspect, the present invention relates to a method of treating an individual having a disease comprising administering to the individual an effective amount of a polypeptide, antibody or composition according to any aspect or embodiment disclosed herein.

In one embodiment of the invention, the disease is selected from the group of: cancer, autoimmune disease, inflammatory disease and infectious disease.

In one embodiment of the invention, the method according to any aspect or embodiment disclosed herein relates to further administering an additional therapeutic agent.

In one embodiment of the invention, the additional therapeutic agent is one or more anti-cancer agent(s) selected from the group consisting of chemotherapeutics (including but not limited to paclitaxel, temozolomide, cisplatin, carboplatin, oxaliplatin, irinotecan, doxorubicin, gemcitabine, 5-fluorouracil, pemetrexed), kinase inhibitors (including but not limited to sorafenib, sunitinib or everolimus), apoptosis-modulating agents (including but not limited to recombinant human TRAIL or birinapant), RAS inhibitors, proteasome inhibitors (including but not limited to bortezomib), histon deacetylase inhibitors (including but not limited to vorinostat), nutraceuticals, cytokines (including but not limited to IFN-γ), antibodies or antibody mimetics (including but not limited to anti-EGFR, anti-IGF-1R, anti-VEGF, anti-CD20, anti-CD38, anti-HER2, anti-PD-1, anti-PD-L1, anti-CTLA4, anti-CD40, anti-CD137, anti-GITR antibodies and antibody mimetics), antibody-drug conjugates.

Kit-of-Parts

It is to be understood that the embodiments described below with reference to a polypeptide or antibody refers to a polypeptide or antibody comprising an Fc region having a CH2-CH3 region of an immunoglobulin and an antigen-binding region, a polypeptide or antibody may also be a multispecific polypeptide or antibody having a first CH2-CH3 region of an immunoglobulin and a first antigen-binding region, and a second polypeptide or antibody having a second Fc region comprising a second CH2-CH3 region of an immunoglobulin and a second antigen-binding region.

The invention also relates to kit-of-parts for simultaneous, separate or sequential use in therapy comprising polypeptides or antibodies described herein. Furthermore, such variants may be obtained according to any method described herein.

In one aspect, the present invention relates to a kit of parts comprising a polypeptide, antibody or composition according to any aspect or embodiment described herein, wherein said polypeptide, antibody or composition is in one or more containers such as vials.

In one embodiment of the present invention, the kit of parts comprises a polypeptide, antibody or a composition according to any aspect or embodiment described herein, for simultaneous, separate or sequential use in therapy.

In another aspect, the present invention relates to use of a polypeptide, an antibody, a composition or kit-of-parts according to any of the embodiments herein described for use in a diagnostic method.

In another aspect, the present invention relates to a diagnostic method comprising administering a polypeptide, antibody, a composition or a kit-of-parts according to any embodiments herein described to at least a part of the body of a human or other mammal.

In another aspect, the present invention relates to use of a polypeptide, an antibody, a composition or kit-of-parts according to any of the embodiments herein described in imaging at least a part of the body of a human or other mammal.

In another aspect, the present invention relates to a method for imaging of at least a part of the body of a human or other mammal, comprising administering a variant, a composition or a kit-of-parts according to any embodiments herein described.

Combinations

Additionally, the invention provides for a preparation of any polypeptide or antibody according to any aspect or embodiment described above, i.e., preparations comprising multiple copies of the polypeptide or antibody. The invention also provides for a composition comprising a polypeptide or antibody according to any aspect or embodiment described above, e.g., a pharmaceutical composition. The invention also provides for the use of any such polypeptide or antibody, preparation, or composition as a medicament.

The invention also provides for combinations of polypeptides or antibodies wherein one polypeptide or antibody comprises at least a first and a second mutation according to the invention, as well as preparations and pharmaceutical compositions of such variant combinations and their use as a medicament. Preferably, the two polypeptides or antibodies bind the same antigen or to different antigens typically expressed on the surface of the same cell, cell membrane, virion and/or other particle.

Conjugates

It is to be understood that the embodiments described below with reference to a polypeptide or antibody refers to a polypeptide or antibody comprising an Fc region having a CH2-CH3 region of an immunoglobulin and an antigen-binding region, a polypeptide or antibody may also be a multispecific polypeptide or antibody having a first CH2-CH3 region of an immunoglobulin and a first antigen-binding region, and a second polypeptide or antibody having a second Fc region comprising a second CH2-CH3 region of an immunoglobulin and a second antigen-binding region.

In one aspect, the present invention relates to a polypeptide or antibody, wherein said variant is conjugated to a drug, toxin or radiolabel, such as wherein the variant is conjugated to a toxin via a linker.

In one embodiment, said variant is part of a fusion protein.

In another aspect, the polypeptide or antibody of the invention is not conjugated at the C-terminus to another molecule, such as a toxin or label. In one embodiment, the variant is conjugated to another molecule at another site, typically at a site which does not interfere with oligomer formation. For example, the antibody variant may, at the other site, be linked to a compound selected from the group consisting of a toxin (including a radioisotope) a prodrug or a drug. Such a compound may make killing of target cells more effective, e.g. in cancer therapy. The resulting variant is thus an immunoconjugate.

Thus, in a further aspect, the present invention provides an antibody linked or conjugated to one or more therapeutic moieties, such as a cytotoxin, a chemotherapeutic drug, a cytokine, an immunosuppressant, and/or a radioisotope. Such conjugates are referred to herein as “immunoconjugates” or “drug conjugates”. Immunoconjugates which include one or more cytotoxins are referred to as “immunotoxins”.

A cytotoxin or cytotoxic agent includes any agent that is detrimental to (e.g., kills) cells. Suitable therapeutic agents for forming immunoconjugates of the present invention include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, maytansine or an analog or derivative thereof, enediyene antitumor antibiotics including neocarzinostatin, calicheamycins, esperamicins, dynemicins, lidamycin, kedarcidin or analogs or derivatives thereof, anthracyclins, mitoxantrone, mithramycin, actinomycin D, 1-dehydrotestosterone, glucocorticoids, procaine, tetracaine, lidocaine, propranolol, and puromycin, antimetabolites (such as methotrexate, 6-mercaptopurine, 6-thioguanine, cytarabine, fludarabin, 5-fluorouracil, decarbazine, hydroxyurea, asparaginase, gemcitabine, cladribine), alkylating agents (such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin; as well as duocarmycin A, duocarmycin SA, CC-1065 (a.k.a. rachelmycin), or analogs or derivatives of CC-1065), dolastatin, pyrrolo[2,1-c][1,4] benzodiazepins (PDBs) or analogues thereof, antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)), anti-mitotic agents (e.g., tubulin-inhibitors) such as monomethyl auristatin E, monomethyl auristatin F, or other analogs or derivatives of dolastatin 10; Histone deacetylase inhibitors such as the hydroxamic acids trichostatin A, vorinostat (SAHA), belinostat, LAQ824, and panobinostat as well as the benzamides, entinostat, CI994, mocetinostat and aliphatic acid compounds such as phenylbutyrate and valproic acid, proteasome inhibitors such as Danoprevir, bortezomib, amatoxins such as α-amantin, diphtheria toxin and related molecules (such as diphtheria A chain and active fragments thereof and hybrid molecules); ricin toxin (such as ricin A or a deglycosylated ricin A chain toxin), cholera toxin, a Shiga-like toxin (SLT-I, SLT-II, SLT-IIV), LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins (PAPI, PAPII, and PAP-S), Momordica charantia inhibitor, curcin, crotin, Sapaonaria officinalis inhibitor, gelonin, mitogellin, restrictocin, phenomycin, and enomycin toxins. Other suitable conjugated molecules include antimicrobial/lytic peptides such as CLIP, Magainin 2, mellitin, Cecropin, and P18; ribonuclease (RNase), DNase I, Staphylococcal enterotoxin-A, pokeweed antiviral protein, diphtherin toxin, and Pseudomonas endotoxin. See, for example, Pastan et al., Cell 47, 641 (1986) and Goldenberg, Calif. A Cancer Journal for Clinicians 44, 43 (1994). Therapeutic agents that may be administered in combination with an antibody of the present invention as described elsewhere herein, such as, e.g., anti-cancer cytokines or chemokines, are also candidates for therapeutic moieties useful for conjugation to an antibody of the present invention.

In one embodiment, the drug conjugates of the present invention comprise an antibody as disclosed herein conjugated to auristatins or auristatin peptide analogs and derivates (U.S. Pat. No. 5,635,483; 5,780,588). Auristatins have been shown to interfere with microtubule dynamics, GTP hydrolysis and nuclear and cellular division (Woyke et al (2001) Antimicrob. Agents and Chemother. 45(12): 3580-3584) and have anti-cancer (U.S. Pat. No. 5,663,149) and anti-fungal activity (Pettit et al., (1998) Antimicrob. Agents and Chemother. 42:2961-2965. The auristatin drug moiety may be attached to the antibody via a linker, through the N (amino) terminus or the C (terminus) of the peptidic drug moiety.

Exemplary auristatin embodiments include the N-terminus-linked monomethyl auristatin drug moieties DE and DF, disclosed in Senter et al., Proceedings of the American Association for Cancer Research. Volume 45, abstract number 623, presented Mar. 28, 2004 and described in US 2005/0238649).

An exemplary auristatin embodiment is MMAE (monomethyl auristatin E). Another exemplary auristatin embodiment is MMAF (monomethyl auristatin F).

In one embodiment, an antibody of the present invention comprises a conjugated nucleic acid or nucleic acid-associated molecule. In one such embodiment, the conjugated nucleic acid is a cytotoxic ribonuclease, an antisense nucleic acid, an inhibitory RNA molecule (e.g., a siRNA molecule) or an immunostimulatory nucleic acid (e.g., an immunostimulatory CpG motif-containing DNA molecule). In another embodiment, an antibody of the present invention is conjugated to an aptamer or a ribozyme.

In one embodiment, antibodies comprising one or more radiolabeled amino acids are provided. A radiolabeled variant may be used for both diagnostic and therapeutic purposes (conjugation to radiolabeled molecules is another possible feature). Non-limiting examples of labels for polypeptides include 3H, 14C, 15N, 35S, 90Y, 99Tc, and 125I, 131I, and 186Re. Methods for preparing radiolabeled amino acids and related peptide derivatives are known in the art, (see, for instance Junghans et al., in Cancer Chemotherapy and Biotherapy 655-686 (2^{nd} Ed., Chafner and Longo, eds., Lippincott Raven (1996)) and U.S. Pat. Nos. 4,681,581, 4,735,210, 5,101,827, 5,102,990 (U.S. RE35,500), U.S. Pat. Nos. 5,648,471 and 5,697,902. For example, a radioisotope may be conjugated by the chloramine-T method.

In one embodiment, the polypeptide or antibody of the present invention is conjugated to a radioisotope or to a radioisotope-containing chelate. For example, the variant can be conjugated to a chelator linker, e.g. DOTA, DTPA or tiuxetan, which allows for the antibody to be complexed with a radioisotope. The variant may also or alternatively comprise or be conjugated to one or more radiolabeled amino acids or other radiolabeled molecule. A radiolabeled variant may be used for both diagnostic and therapeutic purposes. In one embodiment the variant of the present invention is conjugated to an alpha-emitter. Non-limiting examples of radioisotopes include ³H, ¹⁴C, ¹⁵N, ³⁵S, ⁹⁰Y, ⁹⁹Tc, ¹²⁵I, ¹¹¹In, ¹³¹I, ¹⁸⁶Re, ²¹³Bs, ²²⁵Ac and ²²⁷Th.

In one embodiment, the polypeptide or antibody of the present invention may be conjugated to a cytokine selected from the group consisting of IL-2, IL-4, IL-6, IL-7, IL-10, IL-12, IL-13, IL-15, IL-18, IL-23, IL-24, IL-27, IL-28a, IL-28b, IL-29, KGF, IFNα, IFNβ, IFNγ, GM-CSF, CD40L, Flt3 ligand, stem cell factor, ancestim, and TNFα.

Polypeptides or antibodies of the present invention may also be chemically modified by covalent conjugation to a polymer to for instance increase their circulating half-life. Exemplary polymers, and methods to attach them to peptides, are illustrated in for instance U.S. Pat. Nos. 4,766,106, 4,179,337, 4,495,285 and 4,609,546. Additional polymers include polyoxyethylated polyols and polyethylene glycol (PEG) (e.g., a PEG with a molecular weight of between about 1,000 and about 40,000, such as between about 2,000 and about 20,000).

Any method known in the art for conjugating the polypeptide or antibody of the present invention to the conjugated molecule(s), such as those described above, may be employed, including the methods described by Hunter et al., Nature 144, 945 (1962), David et al., Biochemistry 13, 1014 (1974), Pain et al., J. Immunol. Meth. 40, 219 (1981) and Nygren, J. Histochem. and Cytochem. 30, 407 (1982). Such variants may be produced by chemically conjugating the other moiety to the N-terminal side or C-terminal side of the variant or fragment thereof (e.g., an antibody H or L chain) (see, e.g., Antibody Engineering Handbook, edited by Osamu Kanemitsu, published by Chijin Shokan (1994)). Such conjugated variant derivatives may also be generated by conjugation at internal residues or sugars, where appropriate.

The agents may be coupled either directly or indirectly to a polypeptide or antibody of the present invention. One example of indirect coupling of a second agent is coupling via a spacer or linker moiety to cysteine or lysine residues in the bispecific antibody. In one embodiment, a polypeptide or antibody is conjugated to a prodrug molecule that can be activated in vivo to a therapeutic drug via a spacer or linker. In some embodiments, the linker is cleavable under intracellular conditions, such that the cleavage of the linker releases the drug unit from the antibody in the intracellular environment. In some embodiments, the linker is cleavable by a cleavable agent that is present in the intracellular environment (e. g. within a lysosome or endosome or caveola). For example, the spacers or linkers may be cleavable by tumor-cell associated enzymes or other tumor-specific conditions, by which the active drug is formed. Examples of such prodrug technologies and linkers are described in WO02083180, WO2004043493, WO2007018431, WO2007089149, WO2009017394 and WO201062171 by Syntarga B V, et al. Suitable antibody-prodrug technology and duocarmycin analogs can also be found in U.S. Pat. No. 6,989,452 (Medarex), incorporated herein by reference. The linker can also or alternatively be, e.g. a peptidyl linker that is cleaved by an intracellular peptidase or protease enzyme, including but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside the target cells (see e. g. Dubowchik and Walker, 1999, Pharm. Therapeutics 83:67-123). In a specific embodiment, the peptidyl linker cleavable by an intracellular protease is a Val-Cit (valine-citrulline) linker or a Phe-Lys (phenylalanine-lysine) linker (see e.g. U.S. Pat. No. 6,214,345, which describes the synthesis of doxorubicin with the Val-Cit linker and different examples of Phe-Lys linkers). Examples of the structures of a Val-Cit and a Phe-Lys linker include but are not limited to MC-vc-PAB described below, MC-vc-GABA, MC-Phe-Lys-PAB or MC-Phe-Lys-GABA, wherein MC is an abbreviation for maleimido caproyl, vc is an abbreviation for Val-Cit, PAB is an abbreviation for p-aminobenzylcarbamate and GABA is an abbreviation for γ-aminobutyric acid. An advantage of using intracellular proteolytic release of the therapeutic agent is that the agent is typically attenuated when conjugated and the serum stabilities of the conjugates are typically high.

In yet another embodiment, the linker unit is not cleavable and the drug is released by antibody degradation (see US 2005/0238649). Typically, such a linker is not substantially sensitive to the extracellular environment. As used herein, “not substantially sensitive to the extracellular environment” in the context of a linker means that no more than 20%, typically no more than about 15%, more typically no more than about 10%, and even more typically no more than about 5%, no more than about 3%, or no more than about 1% of the linkers, in a sample of variant antibody drug conjugate compound, are cleaved when the variant antibody drug conjugate compound presents in an extracellular environment (e.g. plasma). Whether a linker is not substantially sensitive to the extracellular environment can be determined for example by incubating the variant antibody drug conjugate compound with plasma for a predetermined time period (e.g. 2, 4, 8, 16 or 24 hours) and then quantitating the amount of free drug present in the plasma. Exemplary embodiments comprising MMAE or MMAF and various linker components have the following structures (wherein Ab means antibody and p, representing the drug-loading (or average number of cytostatic or cytotoxic drugs per antibody molecule), is 1 to about 8, e.g. p may be from 4-6, such as from 3-5, or p may be 1, 2, 3, 4, 5, 6, 7 or 8).

Examples where a cleavable linker is combined with an auristatin include MC-vc-PAB-MMAF (also designated as vcMMAF) and MC-vc-PAB-MMAF (also designated as vcMMAE), wherein MC is an abbreviation for maleimido caproyl, vc is an abbreviation for the Val-Cit (valine-citruline) based linker, and PAB is an abbreviation for p-aminobenzylcarbamate.

Other examples include auristatins combined with a non-cleavable linker, such as mcMMAF (mc (MC is the same as mc in this context) is an abbreviation of maleimido caproyl).

In one embodiment, the drug linker moiety is vcMMAE. The vcMMAE drug linker moiety and conjugation methods are disclosed in WO2004010957, U.S. Pat. Nos. 7,659,241, 7,829,531, 7,851,437 and U.S. Ser. No. 11/833,028 (Seattle Genetics, Inc.), (which are incorporated herein by reference), and the vcMMAE drug linker moiety is bound to the antibodies at the cysteines using a method similar to those disclosed in therein.

In one embodiment, the drug linker moiety is mcMMAF. The mcMMAF drug linker moiety and conjugation methods are disclosed in U.S. Pat. No. 7,498,298, U.S. Ser. No. 11/833,954, and WO2005081711 (Seattle Genetics, Inc.), (which are incorporated herein by reference), and the mcMMAF drug linker moiety is bound to the variants at the cysteines using a method similar to those disclosed in therein.

In one embodiment, the polypeptide or antibody of the present invention is attached to a chelator linker, e.g. tiuxetan, which allows for the bispecific antibody to be conjugated to a radioisotope.

In one embodiment, each arm (or Fab-arm) of the polypeptide or antibody is coupled directly or indirectly to the same one or more therapeutic moieties.

In one embodiment, only one arm of the antibody is coupled directly or indirectly to one or more therapeutic moieties.

In one embodiment, each arm of the antibody is coupled directly or indirectly to different therapeutic moieties. For example, in embodiments where the variant is a bispecific antibody and is prepared by controlled Fab-arm exchange of two different monospecific antibodies, e.g. a first and second antibody, as described herein, such bispecific antibodies can be obtained by using monospecific antibodies which are conjugated or associated with different therapeutic moieties.

### Further Uses

It is to be understood that the embodiments described below with reference to a polypeptide or antibody refers to a polypeptide or antibody comprising an Fc region having a CH2-CH3 region of an immunoglobulin and an antigen-binding region, a polypeptide or antibody may also be a multispecific polypeptide or antibody having a first CH2-CH3 region of an immunoglobulin and a first antigen-binding region, and a second polypeptide or antibody having a second Fc region comprising a second CH2-CH3 region of an immunoglobulin and a second antigen-binding region.

In a further aspect, the invention relates to a polypeptide, antibody of the invention as described above for use as a medicament, in particular for use as a medicament for the treatment of diseases or disorders. Examples of such diseases and disorders include, without limitation, cancer and bacterial, viral or fungal infections.

In another aspect, the present invention relates to the polypeptide, antibody, bispecific antibodies, compositions and kit-of-parts described herein, for treatment of a disease, such as cancer.

In another aspect, the present invention relates to a method for treatment of a human disease, comprising administration of a variant, a composition or a kit-of-parts described herein.

In another aspect, the present invention relates to a method for treatment of cancer in a human comprising administration of a variant, a composition or a kit-of-parts.

“Treatment” refers to the administration of an effective amount of a therapeutically active compound of the present invention with the purpose of easing, ameliorating, arresting or eradicating (curing) symptoms or disease states.

An “effective amount” or “therapeutically effective amount” refers to an amount effective, at dosages and for periods of time necessary, to achieve a desired therapeutic result. A therapeutically effective amount of an antibody may vary according to factors such as the disease state, age, sex, and weight of the individual, and the ability of the antibody to elicit a desired response in the individual. A therapeutically effective amount is also one in which any toxic or detrimental effects of the antibody or antibody portion are outweighed by the therapeutically beneficial effects.

### Dosages

It is to be understood that the embodiments described below with reference to a polypeptide or antibody refers to a polypeptide or antibody comprising an Fc region having a CH2-CH3 region of an immunoglobulin and an antigen-binding region, a polypeptide or antibody may also be a multispecific polypeptide or antibody having a first CH2-CH3 region of an immunoglobulin and a first antigen-binding region, and a second polypeptide or antibody having a second Fc region comprising a second CH2-CH3 region of an immunoglobulin and a second antigen-binding region.

Efficient dosages and the dosage regimens for the antibody depend on the disease or condition to be treated and may be determined by the persons skilled in the art. An exemplary, non-limiting range for a therapeutically effective amount of an antibody of the present invention is about 0.1 to 100 mg/kg, such as about 0.1 to 50 mg/kg, for example about 0.1 to 20 mg/kg, such as about 0.1 to 10 mg/kg, for instance about 0.5, about such as 0.3, about 1, about 3, about 5, or about 8 mg/kg.

Polypeptides or antibodies of the present invention may also be administered in combination therapy, i.e., combined with other therapeutic agents relevant for the disease or condition to be treated. Accordingly, in one embodiment, the antibody-containing medicament is for combination with one or more further therapeutic agents, such as a cytotoxic, chemotherapeutic or anti-angiogenic agents. Such combined administration may be simultaneous, separate or sequential.

In a further embodiment, the present invention provides a method for treating or preventing disease, such as cancer, which method comprises administration to a subject in need thereof of a therapeutically effective amount of a variant or pharmaceutical composition of the present invention, in combination with radiotherapy and/or surgery.

### Method of Preparation

It is to be understood that the embodiments described below with reference to a polypeptide or antibody refers to a polypeptide or antibody comprising an Fc region having a CH2-CH3 region of an immunoglobulin and an antigen-binding region, a polypeptide or antibody may also be a multispecific polypeptide or antibody having a first CH2-CH3 region of an immunoglobulin and a first antigen-binding region, and a second polypeptide or antibody having a second Fc region comprising a second CH2-CH3 region of an immunoglobulin and a second antigen-binding region.

The invention also provides isolated nucleic acids and vectors encoding a variant according to any one of the aspects described above, as well as vectors and expression systems encoding the variants. Suitable nucleic acid constructs, vectors and expression systems for antibodies and variants thereof are known in the art, and described in the Examples. In embodiments where the variant comprises not only a heavy chain (or Fc-containing fragment thereof) but also a light chain, the nucleotide sequences encoding the heavy and light chain portions may be present on the same or different nucleic acids or vectors.

The invention also provides a method for producing, in a host cell, a polypeptide or antibody according to any one of the aspects described above, wherein said polypeptide or antibody comprises at least the Fc region of a heavy chain, said method comprising the following steps:
a) providing a nucleotide construct encoding said Fc region of said variant,
b) expressing said nucleotide construct in a host cell, and
c) recovering said antibody variant from a cell culture of said host cell.

In some embodiments, the antibody is a heavy-chain antibody. In most embodiments, however, the antibody will also contain a light chain and thus said host cell further expresses a light-chain-encoding construct, either on the same or a different vector.

Host cells suitable for the recombinant expression of antibodies are well-known in the art, and include CHO, HEK-293, Expi293T, PER-C6, NS/0 and Sp2/0 cells. In one embodiment, said host cell is a cell which is capable of Asn-linked glycosylation of proteins, e.g. a eukaryotic cell, such as a mammalian cell, e.g. a human cell. In a further embodiment, said host cell is a non-human cell which is genetically engineered to produce glycoproteins having human-like or human glycosylation. Examples of such cells are genetically-modified Pichia pastoris (Hamilton et al., Science 301 (2003) 1244-1246; Potgieter et al., J. Biotechnology 139 (2009) 318-325) and genetically-modified Lemna minor (Cox et al., Nature Biotechnology 12 (2006) 1591-1597).

In one embodiment, said host cell is a host cell which is not capable of efficiently removing C-terminal lysine K447 residues from antibody heavy chains. For example, Table 2 in Liu et al. (2008) J Pharm Sci 97: 2426 (incorporated herein by reference) lists a number of such antibody production systems, e.g. Sp2/0, NS/0 or transgenic mammary gland (goat), wherein only partial removal of C-terminal lysines is obtained. In one embodiment, the host cell is a host cell with altered glycosylation machinery. Such cells have been described in the art and can be used as host cells in which to express variants of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as EP1176195; WO03/035835; and WO99/54342. Additional methods for generating engineered glycoforms are known in the art, and include but are not limited to those described in Davies et al., 2001, Biotechnol Bioeng 74:288-294; Shields et al, 2002, J Biol Chem 277:26733-26740; Shinkawa et al., 2003, J Biol Chem 278:3466-3473), U.S. Pat. No. 6,602,684, WO00/61739A1; WO01/292246A1; WO02/311140A1; WO 02/30954A1; Potelligent™ technology (Biowa, Inc. Princeton, N.J.); GlycoMAb™ glycosylation engineering technology (GLYCART biotechnology AG, Zurich, Switzerland); US 20030115614; Okazaki et al., 2004, JMB, 336: 1239-49.

The invention also relates to an antibody obtained or obtainable by the method of the invention described above.

In a further aspect, the invention relates to a host cell capable of producing a polypeptide or antibody of the invention. In one embodiment, the host cell has been transformed or transfected with a nucleotide construct of the invention.

The present invention is further illustrated by the following examples which should not be construed as further limiting.

Sequence Table

| | Immunoglobulin | | UniProtKB |
|---|---|---|---|
| SEQ ID no | subclass (Fc) | Amino acid sequence | Reference |
| SEQ ID 1 | IgG1 | PKDTLMISRTPEVTCVVVDVSHEDPEVKFNWYVD | P01857 |
| | | GVEVHNAKTKPREEQYNSTYRVVSVLTVLHQDWL | (aa 130-330) |
| | | NGKEYKCKVSNKALPAPIEKTISKAKGQPREPQV | |
| | | YTLPPSRDELTKNQVSLTCLVKGFYPSDIAVEWE | |
| | | SNGQPENNYKTTPPVLDSDGSFFLYSKLTVDKSR | |
| | | WQQGNVFSCSVMHEALHNHYTQKSLSLSPGK | |
| SEQ ID 2 | IgG1f | PKDTLMISRTPEVTCVVVDVSHEDPEVKFNWYVD | IgGl |
| | | GVEVHNAKTKPREEQYNSTYRVVSVLTVLHQDWL | allotypic |
| | | NGKEYKCKVSNKALPAPIEKTISKAKGQPREPQV | variant |
| | | YTLPPSREEMTKNQVSLTCLVKGFYPSDIAVEWE | “f” |
| | | SNGQPENNYKTTPPVLDSDGSFFLYSKLTVDKSR | |
| | | WQQGNVFSCSVMHEALHNHYTQKSLSLSPGK | |
| SEQ ID 3 | IgG2 | PKDTLMISRTPEVTCVVVDVSHEDPEVQFNWYVD | P01859 |
| | | GVEVHNAKTKPREEQFNSTFRVVSVLTVVHQDWL | (aa 126-326) |
| | | NGKEYKCKVSNKGLPAPIEKTISKTKGQPREPQV | |
| | | YTLPPSREEMTKNQVSLTCLVKGFYPSDIAVEWE | |
| | | SNGQPENNYKTTPPMLDSDGSFFLYSKLTVDKSR | |
| | | WQQGNVFSCSVMHEALHNHYTQKSLSLSPGK | |
| SEQ ID 4 | IgG3 | PKDTLMISRTPEVTCVVVDVSHEDPEVQFKWYVD | P01860 |
| | | GVEVHNAKTKPREEQYNSTFRVVSVLTVLHQDWL | (aa 177-377) |
| | | NGKEYKCKVSNKALPAPIEKTISKTKGQPREPQV | |
| | | YTLPPSREEMTKNQVSLTCLVKGFYPSDIAVEWE | |
| | | SSGQPENNYNTTPPMLDSDGSFFLYSKLTVDKSR | |
| | | WQQGNIFSCSVMHEALHNRFTQKSLSLSPGK | |
| SEQ ID 5 | IgG4 | PKDTLMISRTPEVTCVVVDVSQEDPEVQFNWYVD | P01861 |
| | | GVEVHNAKTKPREEQFNSTYRVVSVLTVLHQDWL | (aa 127-327) |
| | | NGKEYKCKVSNKGLPSSIEKTISKAKGQPREPQV | |
| | | YTLPPSQEEMTKNQVSLTCLVKGFYPSDIAVEWE | |
| | | SNGQPENNYKTTPPVLDSDGSFFLYSRLTVDKSR | |
| | | WQEGNVFSCSVMHEALHNHYTQKSLSLSLGK | |
| SEQ ID 6 | IgE | SPFDLFIRKSPTITCLVVDLAPSKGTVNLTWSRA | P01854 |
| | | SGKPVNHSTRKEEKQRNGTLTVTSTLPVGTRDWI | (aa 225-428) |
| | | EGETYQCRVTHPHLPRALMRSTTKTSGPRAAPEV | |
| | | YAFATPEWPGSRDKRTLACLIQNFMPEDISVQWL | |
| | | HNEVQLPDARHSTTQPRKTKGSGFFVFSRLEVTR | |
| | | AEWEQKDEFICRAVHEAASPSQTVQRAVSVNPGK | |
| SEQ ID 7 | IgA1 | ALEDLLLGSEANLTCTLTGLRDASGVTFTWTPSS | P01876 |
| | | GKSAVQGPPERDLCGCYSVSSVLPGCAEPWNHGK | (aa 133-353) |
| | | TFTCTAAYPESKTPLTATLSKSGNTFRPEVHLLP | |
| | | PPSEELALNELVTLTCLARGFSPKDVLVRWLQGS | |
| | | QELPREKYLTWASRQEPSQGTTTFAVTSILRVAA | |
| | | EDWKKGDTFSCMVGHEALPLAFTQKTIDRLAGKP | |
| | | THVNVSVVMAEVDGTCY | |
| SEQ ID 8 | IgA2 | ALEDLLLGSEANLTCTLTGLRDASGATFTWTPSS | P01877 |
| | | GKSAVQGPPERDLCGCYSVSSVLPGCAQPWNHGE | (aa 120-340) |
| | | TFTCTAAHPELKTPLTANITKSGNTFRPEVHLLP | |
| | | PPSEELALNELVTLTCLARGFSPKDVLVRWLQGS | |
| | | QELPREKYLTWASRQEPSQGTTTFAVTSILRVAA | |
| | | EDWKKGDTFSCMVGHEALPLAFTQKTIDRLAGKP | |
| | | THVNVSVVMAEVDGTCY | |
| SEQ ID 9 | IgM | SFASIFLTKSTKLTCLVTDLTTYDSVTISWTRQN | P01871 |
| | | GEAVKTHTNISESHPNATFSAVGEASICEDDWNS | (aa 230-452) |
| | | GERFTCTVTHTDLPSPLKQTISRPKGVALHRPDV | |
| | | YLLPPAREQLNLRESATITCLVTGFSPADVFVQW | |
| | | MQRGQPLSPEKYVTSAPMPEPQAPGRYFAHSILT | |
| | | VSEEEWNTGETYTCVAHEALPNRVTERTVDKSTG | |
| | | KPTLYNVSLVMSDTAGTCY | |
| SEQ ID 10 | IgD | AVQDLWLRDKATFTCFVVGSDLKDAHLTWEVAGK | P01880 |
| | | VPTGGVEEGLLERHSNGSQSQHSRLTLPRSLWNA | (aa 176-384) |
| | | GTSVTCTLNHPSLPPQRLMALREPAAQAPVKLSL | |
| | | NLLASSDPPEAASWLLCEVSGFSPPNILLMWLED | |
| | | QREVNTSGFAPARPPPQPGSTTFWAWSVLRVPAP | |
| | | PSPQPATYTCVVSHEDSRTLLNASRSLEVSYVTD | |
| | | HGPMK | |

### EXAMPLES

### Example 1

### Antibody Generation, Production and Purification

### Expression Constructs for Antibodies

For antibody expression, variable heavy (VH) chain and variable light (VL) chain sequences were prepared by gene synthesis (GeneArt Gene Synthesis; ThermoFisher Scientific, Germany) and cloned in pcDNA3.3 expression vectors (ThermoFisher Scientific, US) containing IgG1 heavy chain (HC) and light chain (LC) constant regions. Desired mutations were introduced either by gene synthesis or site directed mutagenesis. Antibodies mentioned in this application have VH and VL sequences derived from previously described CD38 antibody HuMAB 005 (WO2006/099875), DR5 antibodies hDR5-01, hDR5-05 (WO2014/009358), CD52 antibody IgG1-Campath (alemtuzumab, Crowe et al., Clin Exp Immunol. 1992, 87(1):105-110), and CD20 antibodies IgG1-7D8 and IgG1-11B8 (WO2004/035607). In some of the examples the human IgG1 antibody b12, a gp120-specific antibody was used as a negative control (Barbas et al., J Mol Biol. 1993 Apr. 5; 230(3):812-23).

### Transient Expression

Antibodies were expressed as IgG1,κ. Plasmid DNA mixtures encoding both heavy and light chains of antibodies were transiently transfected in Expi293T cells (Life/Thermo Scientific, USA) using 293fectin (Invitrogen, US) essentially as described by Vink et al. (Vink et al., Methods, 65 (1), 5-10 2014).

### Purification and Analysis of Proteins

Antibodies were purified by protein A affinity chromatography. Culture supernatants were filtered over a 0.20 μM dead-end filter and loaded on 5 mL MabSelect SuRe columns (GE Healthcare), washed and eluted with 0.02 M sodium citrate-NaOH, pH 3. The eluates were loaded on a HiPrep Desalting column (GE Healthcare) immediately after purification and the antibodies were buffer exchanged into 12.6 mM NaH₂PO₄, 140 mM NaCl, pH 7.4 buffer (B. Braun or Thermo Fisher). After buffer exchange, samples were sterile filtered over 0.2 μm dead-end filters. Purified proteins were analyzed by a number of bioanalytical assays including capillary electrophoresis on sodium dodecyl sulfate-polyacrylamide gels (CE-SDS) and high-performance size exclusion chromatography (HP-SEC). Concentration was measured by absorbance at 280 nm. Purified antibodies were stored at 2-8° C.

### Example 2

Analysis of the Effect of Mutations that were Previously Shown to Inhibit C1q Binding and CDC in Wild Type Antibodies on the In Vitro CDC Efficacy of IgG-005 Variants with Enhanced Fc-Fc Interactions

The C1q binding center in the CH2 domain of human IgG1 was mapped by alanine substitutions to residues D270, K322, P329 and P331 (Idusogie et al., 2000 J. Immunol.). Mutants D270A, K322A and P329A were able to decrease C1q binding and complement activation by rituximab significantly in a complement concentration-dependent manner (Idusogie et al., 2000 J. Immunol).

IgG hexamerization upon target binding on the cell surface has been shown to support efficient binding of the hexameric structure of C1q resulting in avid C1q binding (Diebolder et al., Science 2014). IgG hexamerization on the cell surface is mediated through intermolecular non-covalent Fc-Fc interactions, and can be enhanced by point mutations in the CH2 domain, including E345R and E430G (Diebolder et al., 2014 Science; De Jong et al., 2015 PloS Biology). Fc-Fc enhancing mutations increase C1q binding avidity on the hexameric antibody structure on the cell surface, while C1q binding affinity is not affected. Therefore, it is unpredictable whether mutations that are described to decrease C1q binding affinity can block CDC by IgG1 antibody variants with a mutation for enhanced Fc-Fc interactions.

Here, we analyzed the effect of introducing a D270A/K322A (AA) double mutation in IgG1-005 variants with stabilized Fc-Fc interactions that are known to enhance complement activation: IgG1-005-E430G and IgG1-005-E345R (WO2013/004842, WO2014/108198) and IgG1-005-E345R/E430G/S440Y (WO2014/006217).

For the CDC assay, 0.1×10⁶ Daudi cells (ATCC #CCL-213™) were pre-incubated in polystyrene round-bottom 96-well plates (Greiner bio-one Cat #650101) with concentration series of purified antibodies in a total volume of 80 μL for 15 min on a shaker at RT. Next, 20 μL normal human serum (NHS; Cat #M0008 Sanquin, Amsterdam, The Netherlands) was added as a source of complement and incubated in a 37° C. incubator for 45 min (20% final NHS concentration; 0.001-10.0 μg/mL final antibody concentrations in 3-fold dilutions). The reaction was stopped by putting the plates on ice before pelleting the cells by centrifugation and replacing the supernatant replaced by 20 μL of 2 μg/mL propidium iodide solution (PI; Sigma Aldrich, Zwijnaarde, The Netherlands). The number of PI-positive cells was determined by FACS analysis on an Intellicyt iQue™ screener (Westburg). The data were analyzed using best-fit values of a non-linear dose-response fit using log-transformed concentrations in GraphPad PRISM 5. The percentage lysis was calculated as (number of PI-positive cells/total number of cells)×100%.

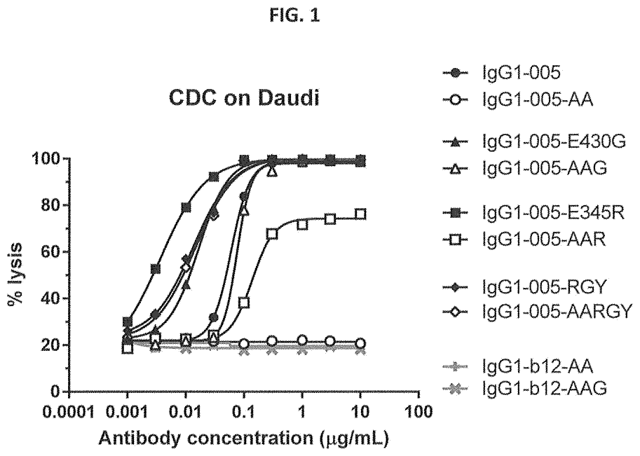

Introduction of the D270A/K322A (AA) double mutation in wild type (WT) IgG1-005 resulted in complete inhibition of CDC on Daudi cells (FIG. 1). In contrast, in the presence of the Fc-Fc interaction enhancing mutations E430G or E345R, introduction of D270A/K322A only had a minor effect on CDC efficacy (FIG. 1): same maximal kill of 100% with EC50 0.01±0.01 (μg/mL±SD) and 0.06±0.02 μg/mL for IgG1-005-E430G and IgG1-005-AA-E430G respectively; maximal kill 100% and 74.3% with EC50 0.01 μg/mL and 0.14 μg/mL for IgG1-005-E345R and IgG1-005-AA-E345R respectively. In the presence of the triple mutation E345R/E430G/S440Y, which results in antibody hexamerization in solution (Diebolder et al., 2014 Science; Wang et al., 2016 Mol. Cell), D270A/K322A did not have any effect on CDC (FIG. 1).

These data show that mutations that inhibited CDC activity of a WT IgG1 antibody were not able to block CDC activity of antibody variants with mutations for enhanced Fc-Fc interactions.

### Example 3

Analysis of the Effect of a Selection of Mutations at Positions D270, K322 and P329 of the C1q Binding Core on the In Vitro CDC Efficacy of IgG1-005 Variants with Enhanced Fc-Fc Interactions

Mutations at positions D270, K322 and P329 of the human IgG1 C1q binding site were designed with the aim to interfere with the protein-protein interactions that are established when C1q is bound to IgG1. Therefore, WT amino acids were substituted by charged amino acids with novel or opposite charges: D270R, K322E, P329D and P329R. These additional mutants were tested for their effect on the CDC efficacy of IgG1-005 variants with the E430G mutation for enhanced Fc-Fc interactions. Concentration series of purified antibodies (0.001-10.0 μg/mL final antibody concentrations in 3-fold dilutions) were tested in an in vitro CDC assay on Daudi cells with 20% NHS as described in Example 2.

| Mutation | Amino acid charge |
|---|---|
| D270A | − → neutral (non-polar) |
| D270R | − → + |
| K322A | + → neutral (non-polar) |
| K322E | + → − |
| P329D | neutral (non-polar) → − |
| P329R | neutral (non-polar) → + |

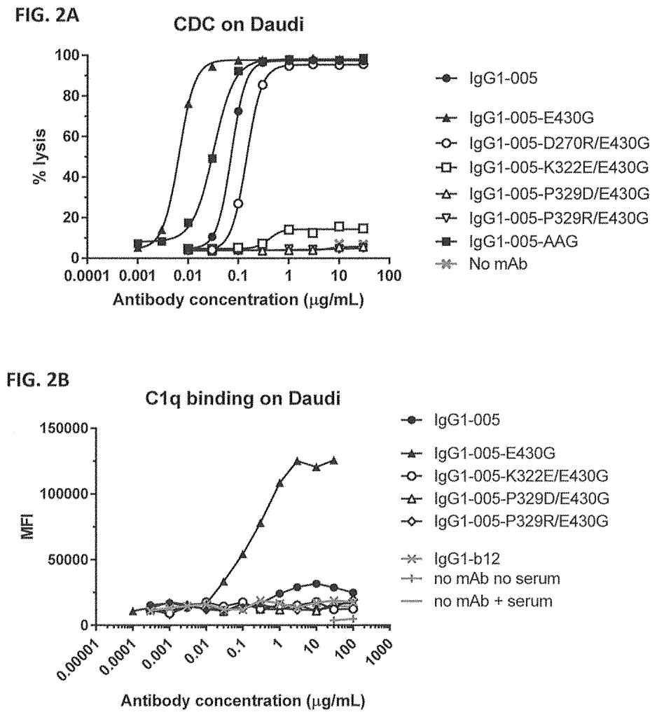

Introduction of the K322E, P329D or P329R mutation strongly inhibited CDC-mediated killing of Daudi cells by IgG1-005-E430G (FIG. 2A). In contrast, introduction of D270R resulted in a decrease in potency and increase in EC50 value (0.005 μg/mL and 0.15 μg/mL for IgG1-005-E430G and IgG1-005-D270R/E430G respectively), but did not decrease maximal kill of Daudi cells by IgG1-005-E430G. Data for D270A/K322A was included as a reference for showing only a minor effect on CDC efficacy by IgG1-005-E430G as described in Example 2.

For the K322E, P329D and P329R mutations that inhibited CDC efficacy of IgG1-005-E430G, the effect on C1q binding to antibodies bound to Daudi cells was measured by FACS analysis. 0.1×10⁶ Daudi cells were incubated for 30 min at 4° C. in 100 μL reactions in polystyrene round-bottom 96-well plates with a concentration series of purified antibodies (0.0003-100.0 μg/mL final antibody concentrations in 3.33-fold dilutions) and 20% C4-depleted serum as a source of C1q. 100 μL FACS buffer (PBS/0.1% BSA/0.01% Na-Azide) was added and cells were pelleted by centrifugation. Cells were washed with 150 μL FACS buffer and incubated for 30 minutes at 4° C. with 50 μL FITC-labeled rabbit anti-HuC1q antibody (DAKO, Cat #F0254; 20 μg/mL final concentration). Cells were washed twice with FACS buffer and resuspended in 30 μL FACS buffer to determine mean fluorescence intensities on an Intellicyt iQue™ screener.

Introduction of the K322E, P329D or P329R mutation inhibited C1q binding to IgG1-005-E430G bound to Daudi cells (FIG. 2B).

Together, these data show that introduction of the K322E, P329D or P329R mutation in IgG1-005-E430G resulted in inhibition of C1q binding and concomitant CDC-mediated killing of Daudi cells.

### Example 4

The Effect of K322X Mutations on the In Vitro CDC Efficacy of IgG1-005 Variants with Enhanced Fc-Fc Interactions

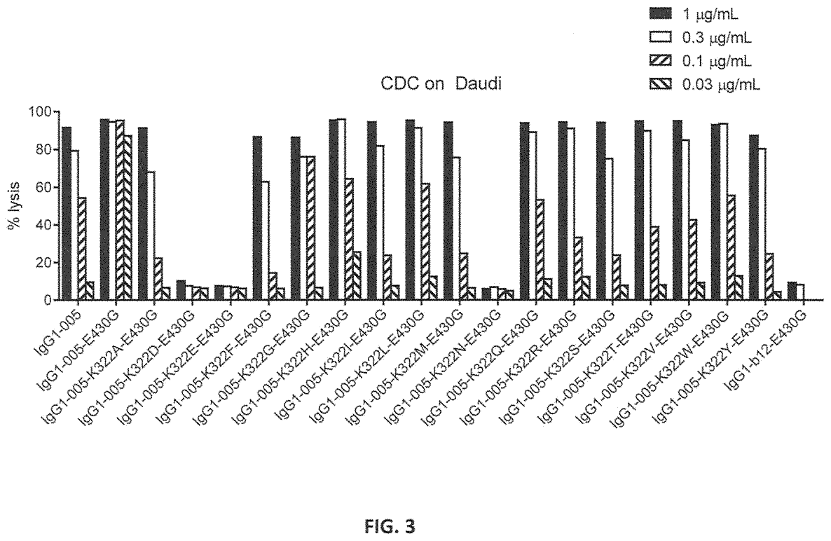

Antibodies were collected by taking the supernatants of transient transfections as described in Example 1. Antibody concentration series (0.001-30.0 μg/mL final concentrations in 3-fold dilutions) were tested in an in vitro CDC assay on Daudi cells with 20% NHS, essentially as described in Example 2. Substitution of the lysine (K) at position 322 to alanine (A), phenylalanine (F), glycine (G), histidine (H), isoleucine (I), leucine (L), methionine (M), glutamine (Q), arginine (R), serine (S), threonine (T), valine (V), tryptophan (W), tyrosine (Y), aspartate (D), glutamate (E) or asparagine (N) in combination with the E430G mutation were tested (FIG. 3).

In this experiment the specific mutations K322D, K322E and K322N were able to block complement activation and CDC by the IgG1-005-E430G with enhanced Fc-Fc interactions.

### Example 5

### Biophysical Characterization of IgG1-005-E430G Variants Containing the Mutation K322D, K322E or K322N

Purified antibody batches of IgG1-005-E430G variants with the K322E, K322D or K322N mutation were analyzed by CE-SDS and HP-SEC.

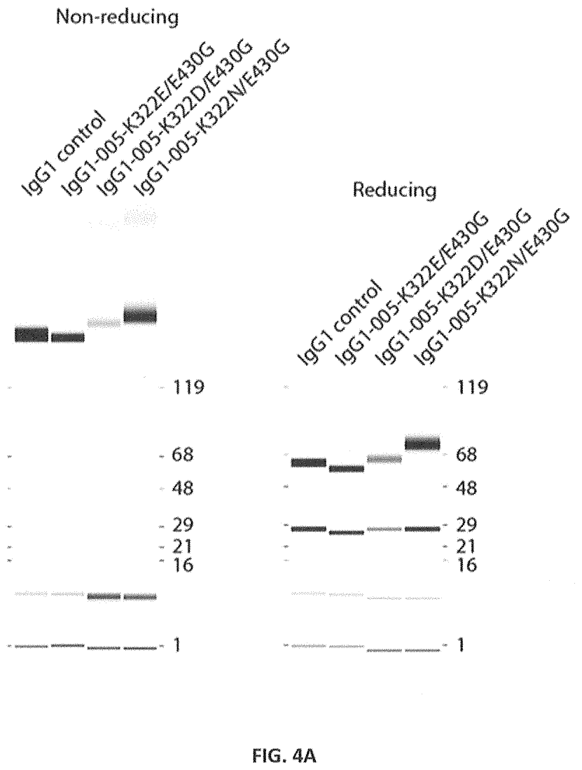

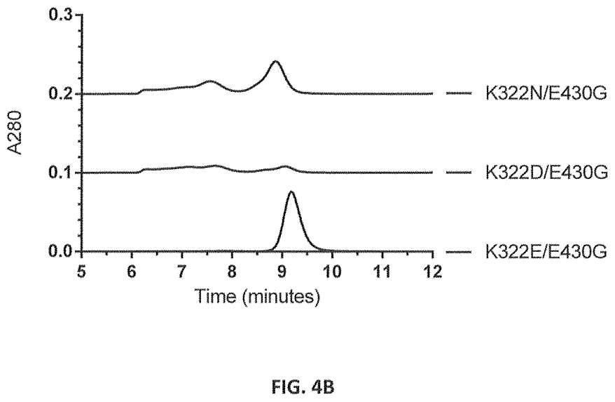

CE-SDS was performed under reducing and non-reducing conditions. Purity and fragmentation of the samples were analyzed using CE-SDS (Caliper Labchip GXII, PerkinElmer) on the Labchip GXII (High Sensitivity protocol) with few modifications. Both nonreduced and reduced samples (addition of DTT) were prepared using the HT Protein Express Reagent Kit (CLS960008) and denatured by incubation at 70° C. for 10 min. Samples were run with the HT antibody analysis 200 high sensitivity settings. Data were analyzed for molecular weight and purity (fraction of total) with Labchip GXII software. FIG. 4A shows that IgG1-005-K322E/E430G displayed behavior comparable to the wild type IgG1 assay control with disulfide-bridged heavy and light chains. A single molecular species with apparent MW of approximately 150 kDa was visible under non-reducing conditions, while under reducing conditions a heavy chain with apparent MW of 50 kDa and light chain of 26 kDa were visible. Antibody variants IgG1-005-K322D/E430G and IgG1-005-K322N/E430G contained higher-molecular weight aggregates under non-reducing conditions that appeared to be resolved after reduction.

HP-SEC fractionation was performed using a Waters Alliance 2975 separation unit (Waters, Etten-Leur, The Netherlands) connected to a TSK HP-SEC column (G3000SWₓₗ; Toso Biosciences, via Omnilabo, Breda, The Netherlands) and a Waters 2487 dual λ absorbance detector (Waters). 50 μL samples containing 1.25 μg/mL protein were separated at 1 mL/min in 0.1 M Na₂SO₄/0.1 M sodium phosphate buffered at pH 6.8. Results were processed using Empower software version 3 and expressed per peak as percentage of total peak area. FIG. 4B shows that while antibody IgG1-005-K322E/E430G eluted overwhelmingly at the elution time expected for a monomeric species (98% monomeric), variants IgG1-005-K322D/E430G (70% aggregated) and IgG1-005-K322N/E430G (43% aggregated) showed considerable amounts of higher molecular weight species. HP-SEC analysis therefore suggested that the double mutant K322E/E430G was more homogeneous in solution than double mutants K322D/E430G and K322N/E430G.

### Example 6

The Effect of P329X Mutations on the In Vitro CDC Efficacy of IgG1-005 Variants with Enhanced Fc-Fc Interactions

The effect of P329X mutations on the in vitro CDC efficacy was tested here on the antibody IgG1-005-E430G which has enhanced CDC. Different concentrations of purified antibodies (range 0.001-30.0 μg/mL final concentrations) were tested in an in vitro CDC assay on Daudi cells with 20% NHS essentially as described in Example 2.

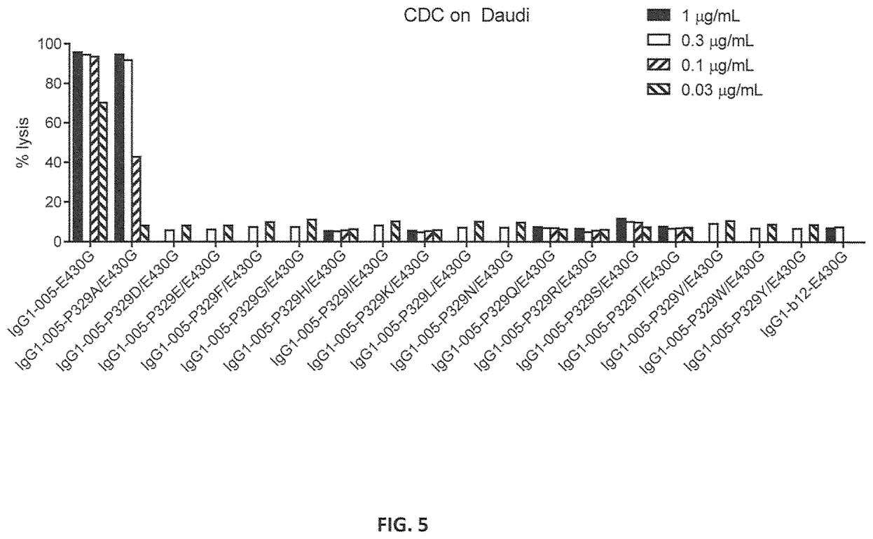

CDC efficacy of IgG1-005-E430G on Daudi cells was completely inhibited by substituting the proline at position P329 to aspartate (D), glutamate (E), phenylalainine (F), glycine (G), histidine (H), isoleucine (I), lysine (K), leucine (L), asparagine (N), glutamine (Q), arginine (R), serine (S), threonine (T), valine (V), tryptophan (W) or tyrosine (Y) (FIG. 5). In contrast, substitution of the proline at position 329 into alanine (A) only partially reduced CDC efficacy with a shift of the EC50 from 0.01 μg/mL for IgG1-005-E430G to 0.11 μg/mL for IgG1-005-P329A/E430G, but with no effect on the maximal kill. These data illustrate that substituting proline at position 329 into another amino acid resulted in either inhibition (in the case of P329D/E/F/G/H/I/K/L/N/Q/R/S/T/V/W/Y) or no inhibition (in the case of P329A) of CDC efficacy by IgG1-005-E430G.

### Example 7

### Biophysical Characterization of IgG1-005-E430G Variants Containing Mutations at Position P329

Purified antibody batches of IgG1-005-E430G variants in which the Proline at position 329 was substituted for any other amino acid except cysteine were analyzed by CE-SDS and HP-SEC.

CE-SDS was performed under reducing and non-reducing conditions as described in Example 5. All tested IgG1-005-E430G antibody variants containing an additional mutation of amino acid P329 displayed behavior similar to the wild type IgG1 assay control antibody, with disulfide-bridged heavy and light chains: a single molecular species with apparent MW of approximately 150 kDa was visible under non-reducing conditions, while under reducing conditions a heavy chain with apparent MW of 50 kDa and light chain of 26 kDa were visible (summarized in Table 1). These data suggest that under denaturing conditions, a monomeric molecule is formed displaying behavior typical of wild type IgG1 antibodies.

HP-SEC fractionation was performed as described in Example 5. The tested IgG1-005-E430G antibody variants that were additionally mutated at amino acid P329 contained variable amounts of higher molecular weight species (Table 1). Variants IgG1-005-P329R/E430G, IgG1-005-P329D/E430G, and IgG1-005-P329T/E430G were essentially homogeneous in solution.

TABLE 1
Biophysical characterization by CE-SDS and HP-SEC
of IgG1-005-P329X/E430G antibody variants, in
which X stand for any amino acid except P or C.

| IgG1-005 | SEC HMW | SEC | IgG | intact HC + |
|---|---|---|---|---|
| variant | (%)¹ | Degradation (%)² | (%)³ | LC (%)⁴ |
| P329Y/E430G | <1 | <1 | 97 | 100 |
| P329T/E430G | 1 | <1 | 96 | 99 |
| P329W/E430G | 1.1 | <1 | 96 | 99 |
| P329V/E430G | 1.2 | <1 | 96 | 99 |
| P329R/E430G | 1.3 | <1 | 95 | 99 |
| P329D/E430G | 1.5 | <1 | 94 | 99 |
| P329L/E430G | 2.5 | <1 | 96 | 99 |
| P329F/E430G | 2.6 | <1 | 97 | 100 |
| P329S/E430G | 2.7 | <1 | 97 | 99 |
| P329N/E430G | 2.9 | <1 | 97 | 99 |
| P329G/E430G | 3.1 | <1 | 97 | 99 |
| P329A/E430G | 3.2 | <1 | 96 | 100 |
| P329I/E430G | 3.5 | <1 | 97 | 99 |
| P329E/E430G | 3.7 | <1 | 96 | 99 |
| P329Q/E430G | 3.7 | <1 | 97 | 99 |
| P329K/E430G | 3.8 | <1 | 97 | 99 |
| P329H/E430G | 3.9 | <1 | 97 | 99 |

### Example 8

### Analysis of the Thermal Stability of IgG1-005-E430G Variants Containing Mutations at Position P329

Purified antibody batches of IgG1-005-E430G variants in which the proline at position 329 was substituted for any other amino acid except cysteine, were analyzed by differential scanning fluorimetry (DSF).

DSF was performed in an iQ5 96-well RT-PCR machine (Bio-Rad) capable of detecting changes in fluorescence intensity caused by binding of the extrinsic dye Sypro-Orange (ThermoFisher-Scientific, S6651) to hydrophobic regions exposed by denatured IgG. A thermal melt curve can be derived from measuring the increasing fluorescence during controlled, stepwise thermal denaturation of the analyzed IgG. Therefore, samples of 5 μL of 0.6 mg/mL IgG protein, mixed with 20 μL of 75 mM Sypro-Orange in either PBS pH 7.4 (B. Braun, Netherlands) or 30 mM NaAc pH 4, were prepared in duplicate. Fluorescence was recorded at temperatures ranging from 25° C. to 95° C., in stepwise increments of 0.5° C. per increment and 15 second duration plus the time necessary to record the fluorescence of all wells.

For each analyzed antibody, the midpoints of the first thermal transition (Tm) observed as a steep increase in fluorescence intensity upon increasing temperature, averaged over both duplicates, are summarized in Table 2. Introduction of P329R or P329K in IgG1-005 and IgG1-005-E430G resulted in a modest increase in the Tm temperature of the antibodies, while introduction of P329D decreased the Tm temperature of both WT IgG1-005 and IgG1-005-E430G. These data suggest that introduction of P329R or P329GK increased the thermal stability of IgG1-005 and IgG1-005-E430G, while P329D decreased the thermal stability of these antibodies.

TABLE 2
DSF analysis of IgG1-005-P329X/E430G antibody variants.

| IgG1-005 | Tm (° C.)² | Tm (° C.)² |
|---|---|---|
| variant¹ | PBS pH 7.4 | Acetate pH 4.0 |
| WT | 70.0 | 57.0 |
| P329D | 66.0 | 52.8 |
| P329K/E430G | 60.8 | 47.5 |
| P329R/E430G | 60.5 | 47.3 |
| E430G | 60.0 | 45.5 |
| P329A/E430G | 59.5 | 45.3 |
| P329S/E430G | 59.0 | 45.0 |
| P329H/E430G | 58.5 | 45.5 |
| P329Q/E430G | 58.5 | 44.0 |
| P329T/E430G | 58.5 | 44.0 |
| P329V/E430G | 58.3 | 44.0 |
| P329L/E430G | 58.0 | 44.0 |
| P329I/E430G | 58.0 | 43.5 |
| P329N/E430G | 58.0 | 43.5 |
| P329G/E430G | 58.0 | 43.0 |
| P329F/E430G | 57.0 | 42.8 |
| P329Y/E430G | 57.0 | 42.0 |
| P329W/E430G | 57.0 | 41.5 |
| P329E/E430G | 57.0 | 41.5 |
| P329D/E430G | 57.0 | 41.0 |

¹IgG1-005 antibody variants were ranked according to decreasing Tm
²Midpoint of the first thermal transition observed upon increasing temperature. Each value represents the average of duplicate measurements.

### Example 9

Effect of Mutations at Position P329 on FcγRIIIa Activation by IgG1-005 Variants with Enhanced Fc-Fc Interactions

The effect on the induction of ADCC was tested for IgG1-005-E430G variants in which proline at position 329 was substituted to any amino acid except cysteine, aspartate, methionine or arginine. Activation of FcγRIIIa-mediated signaling by the IgG1-005-E430G variants containing a mutation at position P329 (P329A/E/F/G/H/I/K/L/N/Q/S/T/V/W/Y) was quantified using the Luminescent ADCC Reporter BioAssay (Promega, Cat #G7015) on Daudi cells, according to the manufacturer's recommendations (Promega, #TM383). As effector cells, the kit contains Jurkat human T cells that are engineered to stably express high affinity FcγRIIIa (V158) and a nuclear factor of activated T cells (NFAT)-response element driving expression of firefly luciferase. Briefly, Daudi cells (5.000 cells/well) were seeded in 384-Wells white OptiPlates (Perkin Elmer Cat #6007290) in ADCC Assay Buffer [RPMI-1640 medium (Lonza, Cat #BE12-115F) supplemented with 3.5% Low IgG Serum] and incubated for 6 hours at 37° C./5% CO2 in a total volume of 30 μL containing antibody concentration series (0.128-2.000 ng/mL final concentrations in 5-fold dilutions) and thawed ADCC Bioassay Effector Cells. After incubating the plates for 15 minutes at room temperature (RT), 30 μL Bio Glo Assay Luciferase Reagent was added and incubated for 5 minutes at RT. Luciferase production was quantified by luminescence readout on an EnVision Multilabel Reader (Perkin Elmer). Luminescence signals were normalized by subtracting with background luminescence signal determined from medium-only samples (no Daudi cells, no antibody, no effector cells).

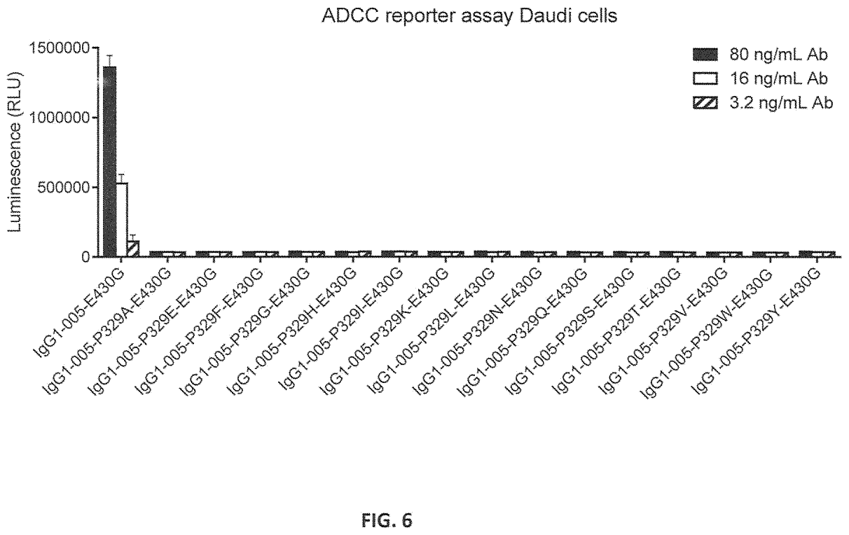

The dose-responsive FcγRIIIa activation by IgG1-005-E430G was completely inhibited by all tested concentrations of all P329X variants (not shown) as illustrated in FIG. 6 for the antibody concentration series 3.2 ng/nL-16 ng/mL-80 ng/mL. These data illustrate that proline at position 329 is essential for binding and activation of FcγRIIIa.

### Example 10

Analysis of the Effect of Mutations at Positions K322 and P329 on the ADCC Efficacy of IgG1-005 Variants with Enhanced Fc-Fc Interactions

CDC-inhibiting mutants of IgG1-005-E430G (described in Example 4 and Example 6) showing favorable biophysical characteristics (described in Example 5, Example 7 and Example 8) were tested for their ADCC efficacy. IgG1-005-E430G variants containing the K322E, P329A, P329D, P329K or P329R mutation were applied in an in vitro ADCC assay on Daudi cells with freshly isolated peripheral blood mononuclear cells (PBMC) from three different healthy donors as effector cells. PBMC were isolated from buffy coats (Sanquin, Amsterdam, The Netherlands) using Lymphocyte Separation Medium (Lonza, Cat #17-829E) for standard Ficoll density centrifugation, according to the manufacturer's instructions. After resuspension of cells in RPMI-1640 medium (Lonza, Cat #BE12-115F) supplemented with 10% Donor Bovine Serum with Iron (DBSI, ThermoFischer, Cat #10371029) and Pen/Strep (Lonza, Cat #DE17-603E), cells were counted by trypan blue exclusion and concentrated to 1×10⁷ cells/mL.

Daudi cells were harvested (5×10⁶ cells/mL), washed (twice in PBS, 1200 rpm, 5 min) and collected in 1 mL RPMI-1640 medium supplemented with 10% DBSI and Pen/Strep, to which 100 μCi ⁵¹Cr (Chromium-51; PerkinElmer, Cat #NEZ030002MC) was added. The mixture was incubated in a shaking water bath for 1 hour at 37° C. After washing of the cells (twice in 50 mL PBS, 1200 rpm, 5 min), the cells were resuspended in RPMI-1640 medium supplemented with 10% DBSI and and Pen/Strep, counted by trypan blue exclusion and diluted to a concentration of 1×10⁵ cells/mL.

For the ADCC experiment, 50 μL ⁵¹Cr-labeled Daudi cells (5.000 cells/well) were pre-incubated with a concentration series (0.3-1.000 ng/mL final concentrations in 3-fold dilutions) of IgG1-005-E430G antibody variants in a total volume of 100 μL RPMI-1640 medium supplemented with 10% DBSI and Pen/Strep in 96-well round-bottom microtiter plates (Greiner Bio-One; Cat #650101). After 20 min at RT, 50 μL PBMC (500.000 cells) were added, resulting in an effector to target ratio of 100:1, and incubated for 4 hours at 37° C./5% CO₂. To determine the maximum amount of cell lysis, 50 μL ⁵¹Cr-labeled Daudi cells (5.000 cells) were incubated with 100 μL 5% Triton-X100. To determine the amount of spontaneous lysis, 5.000 ⁵¹Cr-labeled Daudi cells were incubated in 150 μL medium without any antibody or effector cells. The level of antibody-independent cell lysis was determined by incubating 5.000 Daudi cells with 500.000 PBMCs without antibody. To count the amount of released ⁵¹Cr, plates were centrifuged (1200 rpm, 10 min) and 25 μL of supernatant was transferred to 100 μL Microscint-40 solution (Packard, Cat #6013641) in 96-Wells plates. Plates were sealed and shaken for 15 minutes at 800 rpm and released ⁵¹Cr was counted using a gamma counter. The measured counts per minute (cpm) were used to calculate the percentage of antibody-mediated lysis as follows: (cpm sample−cpm Ab-independent lysis)/(cpm max. lysis−cpm spontaneous lysis)×100%.

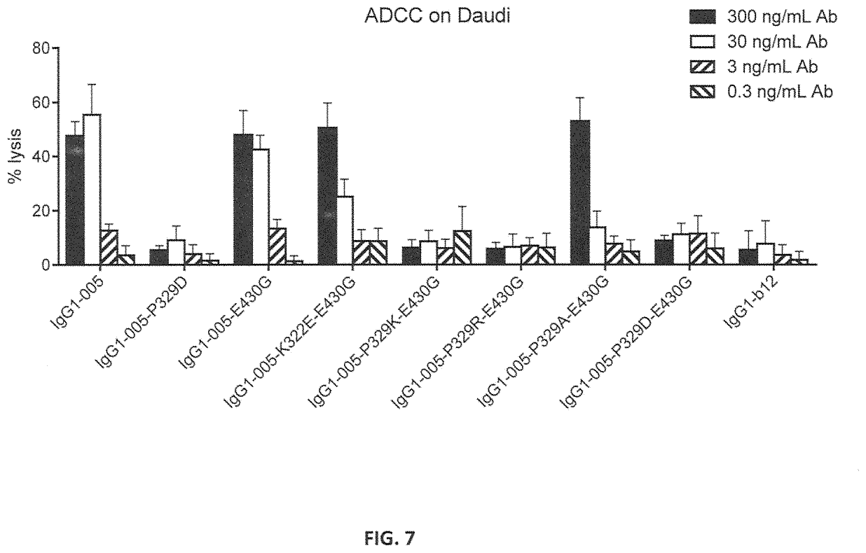

The dose-responsive ADCC-mediated killing of Daudi cells by IgG1-005-E430G was completely inhibited by introducing the P329D, P329K or P329R mutation as illustrated for the antibody concentration series 0.3 ng/mL-3 ng/mL-30 ng/mL-300 ng/mL in FIG. 7. In contrast, the IgG1-005-E430G variants with the K322E or P329A mutation retained considerable ADCC efficacy on Daudi cells.

In summary of the CDC data described in Example 4 and Example 6, the ADCC reporter data described in Example 9 and the in vitro ADCC data described in this Example, introduction of the P329D, P329K or P329R mutation resulted in inhibition of both CDC and ADCC activity of IgG1-005-E430G, despite the enhancing effect of the E430G mutation on Fc-Fc interactions and hexamerization upon target binding on the cell surface. In contrast, the K322E and P329A mutations resulted in CDC inhibition, while retaining ADCC efficacy by IgG1-005-E430G.

### Example 11

The P329D Mutation is Generally Applicable to Inhibit Complement Activation and CDC by IgG1 Antibodies with Mutations for Enhanced Fc-Fc Interactions

Example 3 and Example 6 describe that introduction of the P329D mutation in an anti-CD38 mAb IgG1-005 variant containing the E430G mutation for enhanced Fc-Fc interactions, resulted in complete inhibition of CDC activity on Daudi cells. Next, it was tested if introduction of the P329D mutation had the same effect on IgG1-005 variants containing other Fc-Fc-enhancing mutations. Therefore, the P329D mutation was introduced in IgG1-005 variants with the E345R, E345K or E345R/E430G/S440Y (RGY) mutation(s) and tested on Daudi cells for C1q binding and in an in vitro CDC assay.

C1q binding to antibodies bound to Daudi cells was measured by FACS analysis as described in Example 3. For the CDC assay, antibody concentration series (0.0003-100.0 μg/mL final concentrations in 3.33-fold dilutions) were tested on Daudi cells with 20% NHS as described in Example 2.

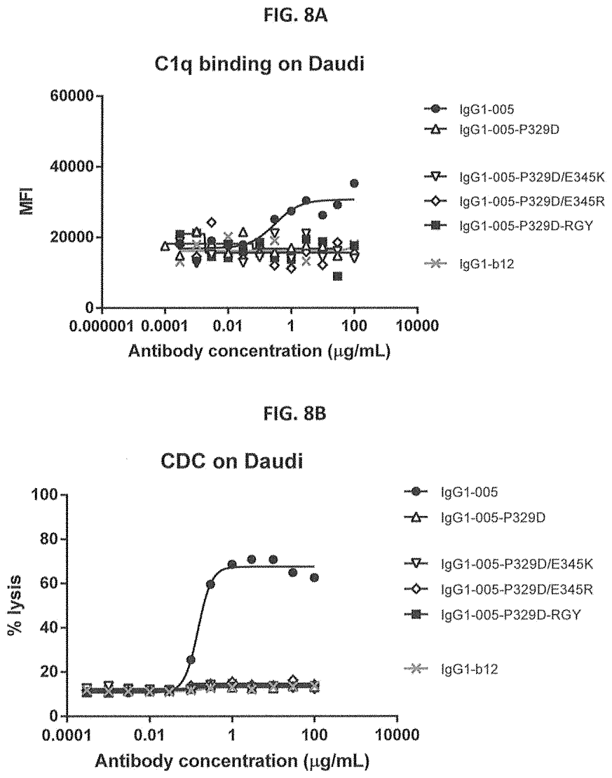

Introduction of the P329D mutation resulted in complete inhibition of C1q binding (FIG. 8A) and CDC efficacy (FIG. 8B) on Daudi cells by IgG1-005 variants with either the E345K, E345R or E345R/E430G/S440Y mutations for enhanced Fc-Fc interactions.

The data with the E345K, E345R and RGY mutations presented in this example, together with the E430G data described in Example 3, illustrates that C1q binding and CDC efficacy by IgG1-005 antibodies with a mutation for enhanced Fc-Fc interactions can be generally inhibited by introduction of the P329D mutation.

### Example 12

### Biophysical Characterization of Hexameric IgG1-005-E345R/E430G/S440Y Variants Containing the K322E or P329D Mutation

To study the effect of K322E and P329D on IgG1 hexamerization, we made use of the triple mutant IgG1-005-E345R/E430G/S440Y, in which the three Fc-Fc interaction-enhancing mutations E345R, E430G and S440Y (RGY) are combined and for which it was shown that it forms antibody hexamers in solution (Diebolder et al., Science 2014). K322E or P329D was introduced in IgG1-005-RGY generating IgG1-005-K322E/E345R/E430G/S440Y (IgG1-005-ERGY) and IgG1-005-P329D/E345R/E430G/S440Y (IgG1-005-DRGY) and the effect on antibody hexamerization was analyzed by CE-SDS, HP-SEC and native mass spectrometry.

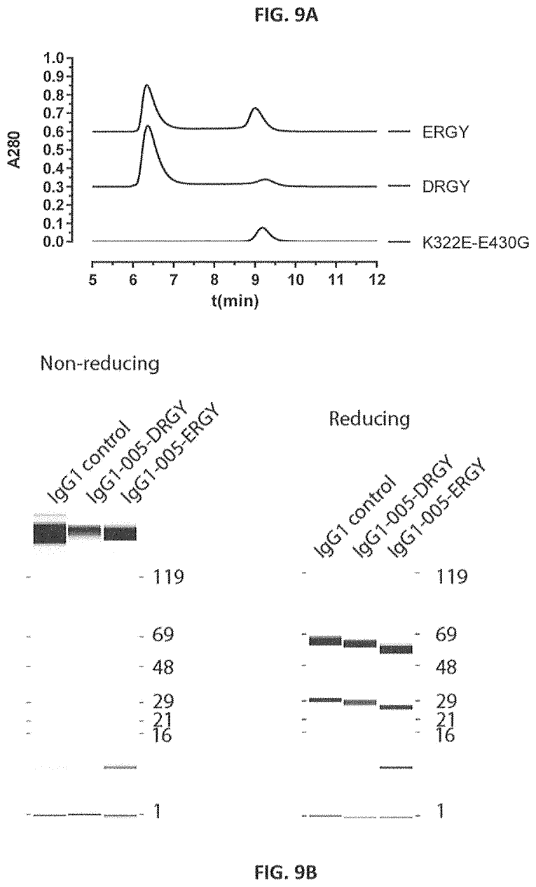

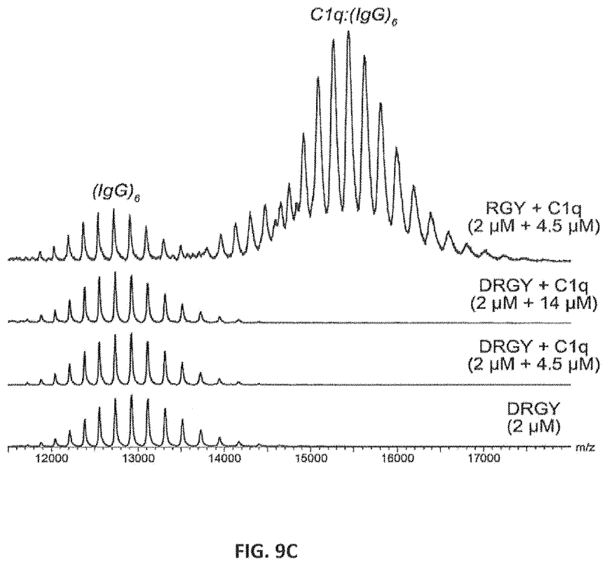

HP-SEC fractionation was performed as described in Example 5. Consistent with the behavior observed for IgG1-005-RGY (Diebolder et al., Science 2014), both IgG1-005-ERGY and IgG1-005-DRGY retained their capability to oligomerize in solution (FIG. 9A). Two peaks were observed corresponding to oligomer (elution time ˜6.3 minutes) and monomer (elution time ˜9-9.3 minutes), with intermediate intensity probably caused by dynamic exchange between the oligomeric and monomeric state.

The fraction oligomer in IgG1-005-ERGY was determined at 58.4%, while 31.4% was monomeric; 10.2% eluted as intermediate species. The fraction oligomer in IgG1-005-DRGY was determined at 78.8%, while the fraction monomer was 12.5%; 8.7% intermediate species were observed.

CE-SDS was performed under reducing and non-reducing conditions. In accordance with the results observed for IgG1-005-RGY (Diebolder et al., Science 2014), both IgG1-005-ERGY and IgG1-005-DRGY displayed a single molecular species with apparent MW of approximately 150 kDa under non-reducing conditions, while under reducing conditions, a heavy chain with apparent MW of 50 kDa and light chain of 26 kDa were visible (FIG. 9B). These data showed that IgG1-005-ERGY and IgG1-005-DRGY behaved similar to the WT monomeric IgG1 assay control antibody, and indicate that hexamerization is disturbed under denaturing CE-SDS conditions, consistent with non-covalent Fc-Fc interactions.

Native mass spectrometry analysis of 2 μM IgG1-005-DRGY, in the absence or presence of excess C1q, buffered in 150 mM ammonium acetate pH 7.5 was conducted with a modified LCT time-of-flight (Waters, UK) mass spectrometer adjusted for optimal performance in high mass detection. Samples were sprayed from borosilicate glass capillaries mounted to a standard static nanospray source. Data analysis was conducted with MassLynx (Waters, UK) and Origin Pro (Origin Lab, USA) software. IgG1-005-DRGY formed hexamers, as observed for IgG1-005-RGY (FIG. 9). Addition of C1q to IgG1-005-DRGY hexamers did not yield detectable C1q binding, while IgG1-005-RGY readily bound C1q under an equivalent condition (FIG. 9C).

In summary, the biophysical analyses described in this example indicate that introduction of the C1q binding inhibiting mutation P329D or K322E did not block hexamerization of IgG1-005-RGY in solution (HP-SEC, native MS), but completely abolished C1q binding (native MS). Moreover, the oligomers that were formed by antibody variants IgG1-005-ERGY and IgG1-005-DRGY in solution were formed by non-covalent interactions (CE-SDS) in agreement with the Fc-Fc interactions described for IgG1-005-RGY (Diebolder et al., Science 2014).

### Example 13

Analysis of the Efficacy to Induce Killing by Agonistic DR5 Antibodies with Enhanced Fc-Fc Interactions in the Presence of the P329D Mutation

Agonistic death receptor 5 (DR5) antibodies can induce killing of DR5-positive tumor cells by activation of the extrinsic apoptosis pathway through DR5 hyperclustering, resulting in recruitment of the adaptor protein Fas-associated protein with death domain (FADD) to the intracellular DR5 death domain, which in turn leads to binding and activation caspase-8 and formation of the DISC (death-inducing signaling complex) that initiates apoptosis. To show that Fc-Fc interactions are involved in the killing by a combination of DR5 antibodies containing E430G mutation for enhanced Fc-Fc interactions (IgG1-hDR5-01-G56T-E430G+IgG1-hDR5-05-E430G), we made use of the 13-residue peptide DCAWHLGELVWCT (DeLano et al., Science 2000 Feb. 18; 287(5456):1279-83) that binds the Fc in a region containing the core amino acids in the hydrophobic patch that are involved in Fc-Fc interactions (Diebolder et al., Science. 2014 Mar. 14; 343(6176):1260-3). A viability assay on BxPC-3 cells was performed in presence or absence of the DCAWHLGELVWCT peptide. Adherent BxPC-3 (ATCC, CRL-1687) cells were harvested by trypsinization and passed through a cell strainer. Cells were pelleted by centrifugation for 5 minutes at 1,200 rpm and resuspended in culture medium at a concentration of 0.5×10⁵ cells/mL [RPMI 1640 with 25 mM Hepes and L-Glutamine (Lonza Cat nr BE12-115F)+10% DBSI (Life Technologies Cat nr 10371-029)+Pen/Strep (Lonza Cat nr DE17-603E)]. 100 μL of the single cell suspensions (5,000 cells per well) were seeded in polystyrene 96-well flat-bottom plates (Greiner Bio-One, Cat nr 655182) and incubated overnight at 37° C. Culture medium was removed and replaced by 100 μL culture medium containing 100 μg/mL of the Fc-binding DCAWHLGELVWCT peptide, a non-specific control peptide GWTVFQKRLDGSV, or no peptide. Next, 50 μL of the antibody combination IgG1-hDR5-01-G56T-E430G+IgG1-hDR5-05-E430G (833 ng/mL final concentration) was added and incubated for 3 days at 37° C. To determine maximal killing, a sample was incubated with 5 μM staurosporine (Sigma Aldrich, Cat nr S6942). The percentage viable cells was determined in a CellTiter-Glo luminescent cell viability assay (Promega, Cat nr G7571) that quantifies the ATP present, which is an indicator of metabolically active cells. From the kit, 20 μL luciferin solution reagent was added per well and mixed by shaking the plate for 2 minutes at 500 rpm. Next, plates were incubated for 1.5 hours at 37° C. 100 μL supernatant was transferred to a white OptiPlate-96 (Perkin Elmer, Cat nr 6005299) and luminescence was measured on an EnVision Multilabel Reader (PerkinElmer). Data were analyzed and plotted using non-linear regression (sigmoidal dose-response with variable slope) using GraphPad Prism software. The percentage viable cells was calculated using the following formula: % viable cells=[(luminescence antibody sample−luminescence staurosporine sample)/(luminescence no antibody sample−luminescence staurosporine sample)]*100.

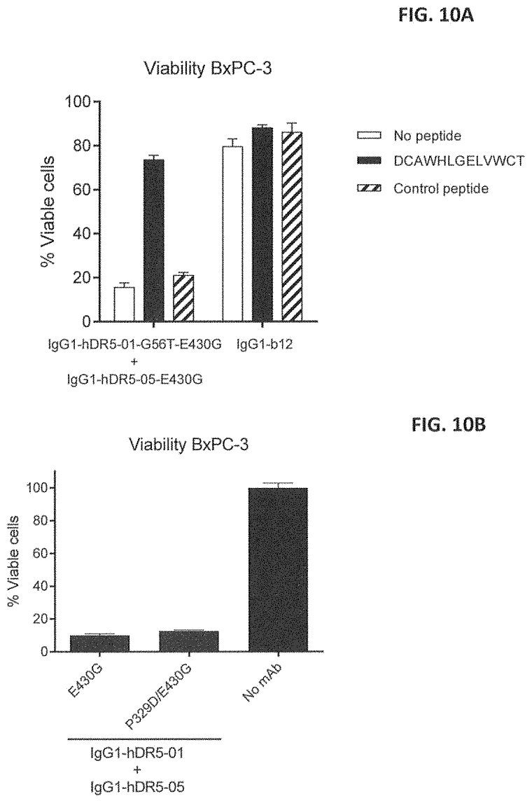

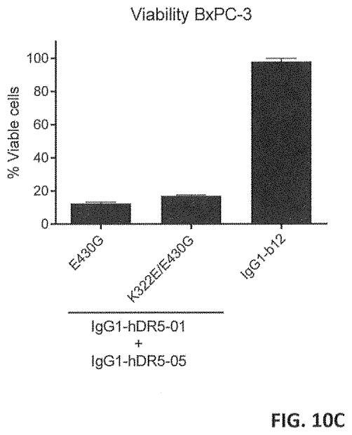

The capacity of the antibody combination IgG1-hDR5-01-G56T-E430G+IgG1-hDR5-05-E430G to induce killing of BxPC-3 cells was strongly inhibited by 100 μg/mL Fc-binding DCAWHLGELVWCT peptide (FIG. 10A). These data indicate that Fc-Fc interactions are required for the antibody combination IgG1-hDR5-01-G56T-E430G+IgG1-hDR5-05-E430G with Fc-Fc-enhancing mutation to induce DR5 clustering on the cell surface of cancer cells and induction of apoptosis.

Next, a viability assay was performed to study the effect of introducing the mutation P329D on the DR5 clustering and induction of apoptosis by agonistic DR5 antibodies with an E430G mutation for enhanced Fc-Fc interactions. A viability assay on BxPC-3 cells was performed, essentially as described above. Briefly, BxPC-3 cells that were allowed to adhere overnight (5,000 cells per well) were incubated for 3 days at 37° C. with 5 μg/mL or 10 μg/mL final antibody concentration in a total volume of 150 μL. The percentage viable cells were determined in a CellTiter-Glo luminescent cell viability assay.

After introduction of the P329D (FIG. 10B) or K322E (FIG. 10C) mutation, saturating antibody concentrations of the combination IgG1-hDR5-01-E430G+IgG1-hDR5-05-E430G with the E430G mutation for enhanced Fc-Fc interactions, were still able to induce killing of BxPC-3 cells.

Together, these data illustrate that the P329D and K322E mutations did not block Fc-Fc interactions that are required for clustering and induction of apoptosis upon DR5 binding on the target cells by saturating concentrations of the agonistic DR5 antibodies with the E430G mutation for enhanced Fc-Fc interactions.

### Example 14

Glycosylation Profiling of IgG1-005 Variants with Enhanced Fc-Fc Interactions Containing the K322E, P329D or P329R Mutation

N-linked glycans of purified antibodies IgG1-005-K322E/E430G, IgG1 P329D/E430G and IgG1-005-P329R/E430G were analyzed by Mass Spectrometry.

IgG samples were incubated with DTT for 1 h at 37° C. Next, samples were desalted on an Ultimate 3000 UPLC system (Dionex) by using a 10 min block gradient on a Proswift RP-4H 1×250 mm column (Thermo Scientific) at 60° C. with MilliQ water (Eluent A) and LC-MS grade acetonitrile (eluent B), both with 0.05% formic acid (Fluke). The UPLC system was coupled to a Q-Exactive Plus Orbitrap MS system (Thermo Scientific) equipped with an electrospray ionization HESI source. Prior to analysis, an 800-3000 m/z scale was calibrated using LTQ Velos ESI positive calibration mix. Recorded mass spectra were deconvoluted with Protein Deconvolution software (Thermo Scientific), and used for quantitation of the relative abundance of individual N-linked glycans.

Antibody variants IgG1-005-K322E/E430G, IgG1-005-P329D/E430G and IgG1-005-P329R/E430G all displayed glycosylation profiles similar to those commonly observed for IgG1 antibodies expressed in EXPI293 cells, with low levels of mannose-5 or charged species, a high level of fucosylation, and between 10% and 30% of galactosylated species (Table 3). These data suggest that mutations K322E, P329D and P329R did not materially impact the glycosylation profile of IgG1-005-E430G.

TABLE 3
N-linked glycan distribution of IgG1-005-E430G variants

| | IgG1-005- | IgG1-005- | IgG1-005- | |
|---|---|---|---|---|
| | P329R/ | P329D/ | K322E/ | IgG1-005- |
| | E430G | E430G | E430G | E430G |
| Neutral peaks | 100% | 100% | 100% | 100% |
| Charged | ND¹ | ND | ND | ND |
| peaks | | | | |
| G0F | 70.7% | 61.0% | 55.9% | 70.7% |
| G1F | 24.9% | 32.8% | 35.0% | 24.0% |
| G2F | ND | 3.0% | 4.3% | ND |
| Man5 | ND | ND | ND | ND |
| Fucosylation | 100% | 100% | 100% | 100% |
| Galactosylation | 13.0% | 20.1% | 22.9% | 12.7% |
| Peaks | 100% | 100% | 97.9% | 100% |
| identified | | | | |

### Example 15

Pharmacokinetic (PK) Analysis of IgG-005 Variants with Enhanced Fc-Fc Interactions Containing the K322E, P329D or P329R Mutation

The effect of the K322E, P329D and P329R mutation on the clearance rate of IgG1-005-E430G was studied in a PK experiment in SCID mice. The clearance rate of IgG1-005-K322E/E430G, IgG1-005-P329D/E430G and IgG1-005-P329R/E430G was compared to that of IgG1-005-E430G without CDC-inhibiting mutation and WT IgG1-005 without the E430G mutation for enhanced Fc-Fc interactions.

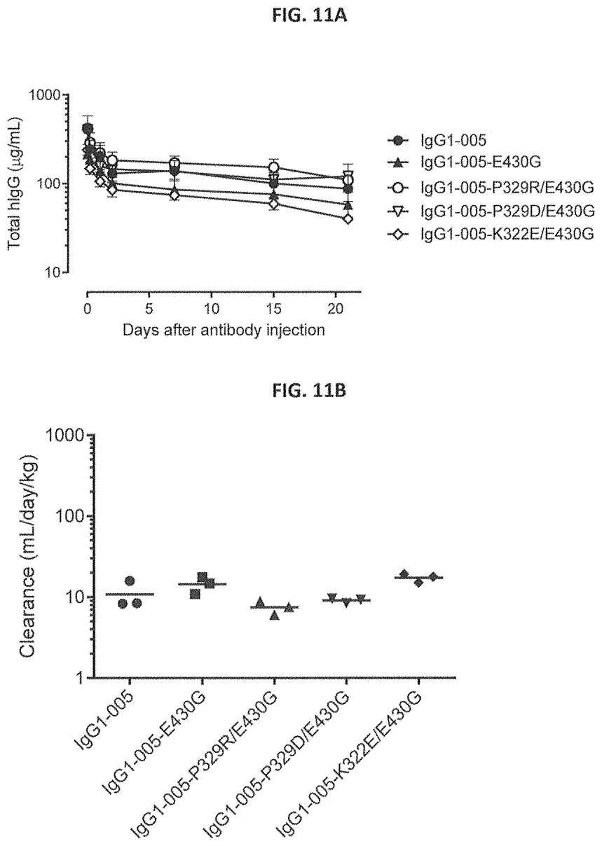

The mice in this study were housed in the Central Laboratory Animal Facility (Utrecht, The Netherlands) and handled in accordance with good animal practice as defined by FELASA, in an AAALAC and ISO 9001:2000 accredited animal facility (GDL). All experiments were performed in compliance with the Dutch animal protection law (WoD) translated from the directives (2010/63/EU) and approved by the Utrecht University animal ethics committee. 11-12 weeks old female SCID (C.B-17/IcrHan@Hsd-Prkdc<scid, Envigo) mice (3 mice per group) were injected intravenously with 500 μg antibody (25 mg/kg) in a 210 μL (for IgG1-005-K322E/E430G) or 200 μL (for the other batches) injection volume. 50-100 μL blood samples were collected from the saphenous vein at 10 minutes, 4 hours, 1 day, 2 days, 7 days, 14 days and 21 days after antibody administration. Blood was collected into heparin-containing vials and centrifuged for 10 minutes at 14,000 g. 20 μL plasma samples were diluted with 980 μL PBST (PBS supplemented with 0.05% Tween 20) supplemented with 0.2% bovine serum albumin (BSA) and stored at −20° C. until determination of antibody concentrations. Total human IgG concentrations were determined using a sandwich ELISA. Mouse anti-human IgG-kappa mAb clone MH16 (CLB Sanquin, Cat #M1268) was used as capturing antibody and coated in 100 μL overnight at 4° C. to 96-well Microlon ELISA plates (Greiner, Germany) at a concentration of 2 μg/mL in PBS. Plates were blocked by incubating on a plate shaker for 1h at RT with PBS supplemented with 0.2% BSA. After washing, 100 μL of the diluted plasma samples were added and incubated on a plate shaker for 1h at RT. Plates were washed three times with 300 μL PBST and subsequently incubated on a plate shaker for 1h at RT with 100 μL peroxidase-labeled goat anti-human IgG immunoglobulin (#109-035-098, Jackson, West Grace, PA; 1:10.000 in PBST supplemented with 0.2% BSA). Plates were washed again three times with 300 μL PBST before incubation for 15 minutes at RT with 100 μL substrate 2,2′-azino-bis (3-ethylbenzthiazoline-6-sulfonic acid) [ABTS; Roche, Cat #11112 422001; 1 tablet in 50 mL ABTS buffer (Roche, Cat #11112 597001)] protected from light. The reaction was stopped by adding 100 μL 2% oxalic acid and incubation for 10 minutes at RT. Absorbance was measured in a microplate reader (Biotek, Winooski, Vt.) at 405 nm. Concentration was calculated by using the injected material as a reference curve. As a plate control human myeloma protein containing IgG, (The binding site, UK) was included. Human IgG concentrations (in μg/mL) were plotted (FIG. 11A) and Area under the curve (AUC) was calculated using Graphpad prism 6.0. Clearance until the last day of blood sampling (day 21) was determined by the formula D*1.000/AUC, in which D is the dose of injection (25 mg/kg) (FIG. 11B).

The CDC-inhibited mutants IgG1-005-K322E/E430G, IgG1-005-P329D/E430G and IgG1-005-P329R/E430G all showed clearance rates in the same range as IgG1-005-E430G and WT IgG1-005 (FIG. 11). These data indicate that the clearance rate of the IgG1-005-E430G antibody with enhanced Fc-Fc interactions was not affected by the K322E (inhibiting CDC but retaining ADCC efficacy) or P329D and P329R (inhibiting both CDC and ADCC efficacy).

### Example 16

The Effect of P329X Mutations on the In Vitro CDC Efficacy of IgG1-005 Variants with Enhanced Fc-Fc Interactions

The effect of P329X mutations on the in vitro CDC efficacy was tested here on the antibody IgG1-005-E430G which has enhanced CDC compared to IgG1-005. Different concentrations of purified antibodies (range 0.001-30.0 μg/mL final concentrations) were tested in an in vitro CDC assay on Daudi cells with 20% NHS essentially as described in Example 2.

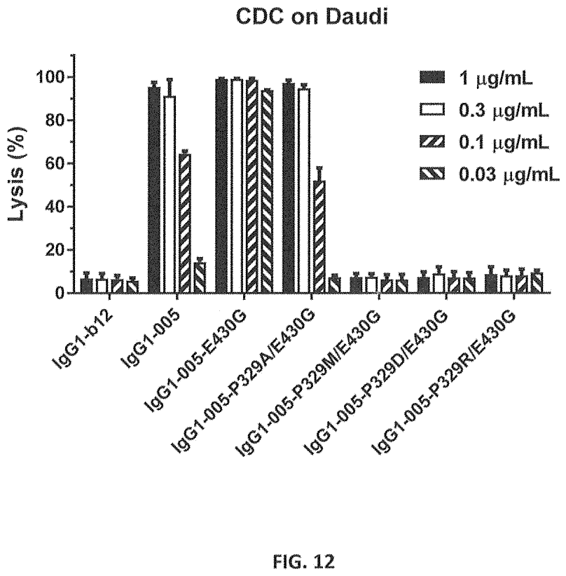

CDC efficacy of IgG1-005-E430G on Daudi cells was completely inhibited by substituting the proline at position P329 to methionine (M), aspartate (D), or arginine (R) (FIG. 12). In contrast, substitution of the proline at position 329 into alanine (A) only partially reduced CDC efficacy with a shift of the EC50 from 0.01 μg/mL for IgG1-005-E430G to 0.10 μg/mL for IgG1-005-P329A/E430G, but with no effect on the maximal kill. These data illustrate that substituting proline at position 329 into another amino acid resulted in either inhibition (in the case of P329M/D/R) or no inhibition (in the case of P329A) of CDC efficacy by IgG1-005-E430G

### Example 17

The Effect of P329R and P329D Mutations on the In Vitro CDC Efficacy of Campath IgG Isotype Variants with Enhanced Fc-Fc Interactions

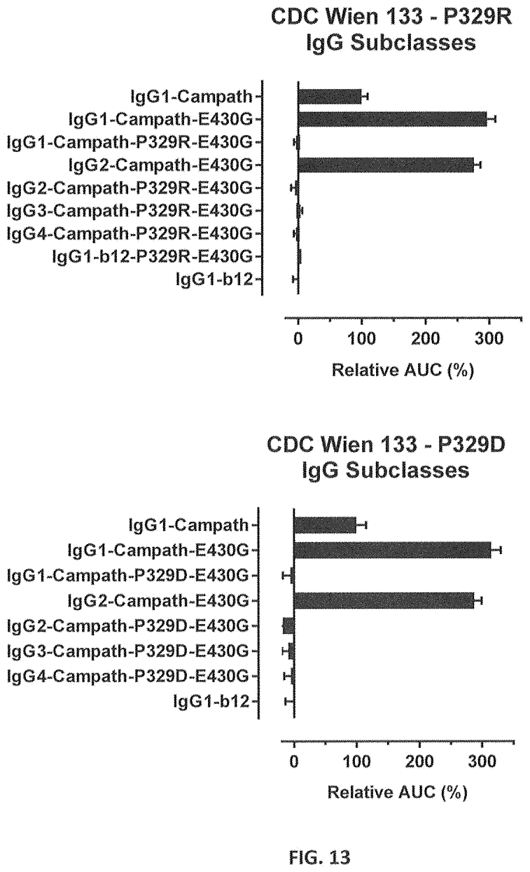

The effect of P329R and P329D mutations on in vitro CDC efficacy was tested using different IgG isotype variants of the antibody IgG1-Campath-E430G, which has enhanced CDC compared to IgG1-Campath (FIG. 13). Different concentrations of purified antibodies (range 0.001-30.0 μg/mL final concentrations) were tested in an in vitro CDC assay on Wien 133 cells with 20% NHS essentially as described in Example 2. The area under the dose-response curves of three experimental replicates was calculated using a log transformed concentration axis with GraphPad Prism 7.02 and normalized relative to cell lysis measured for isotype control antibody IgG1-b12 (0%) and IgG1-Campath (100%).

While the area under the CDC dose response curve on Wien 133 cells of IgG1-Campath-E430G increased approximately 3-fold compared to WT, CDC activity was reduced to background levels by substituting the proline at position 329 to arginine (R) or to aspartic acid (D) (FIG. 13). Similarly, CDC by IgG2-Campath-E430G was reduced to background levels upon introduction of mutations P329R or P329D. Also IgG3 and IgG4 isotype variants containing both an E430G and either a P329R or P329D mutation failed to show CDC lysis above background levels.

These data illustrate that substituting proline at position 329 into arginine or aspartic acid resulted in efficient inhibition of CDC efficacy by IgG1, IgG2, IgG3, and IgG4 isotype variants of IgG1-Campath-E430G.

### Example 18

The Effect of Mutation K322E on the In Vitro CDC Efficacy of Campath IgG Isotype Variants with Enhanced Fc-Fc Interactions

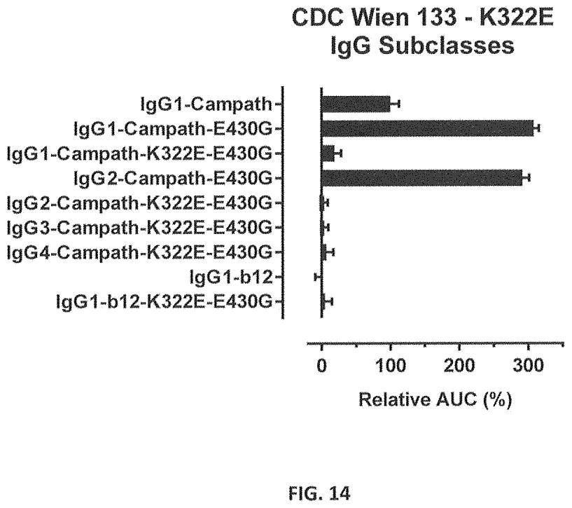

The effect of mutation K322E on in vitro CDC efficacy was tested using different IgG isotype variants of the antibody IgG1-Campath-E430G, which has enhanced CDC compared to IgG1-Campath (FIG. 14). Different concentrations of purified antibodies (range 0.001-30.0 μg/mL final concentrations) were tested in an in vitro CDC assay on Wien 133 cells with 20% NHS essentially as described in Example 2. The area under the dose-response curves of three experimental replicates was calculated using a log transformed concentration axis with GraphPad Prism 7.02 and normalized relative to cell lysis measured for isotype control antibody IgG1-b12 (0%) and IgG1-Campath (100%).

While the area under the CDC dose response curve on Wien 133 cells of IgG1-Campath-E430G increased approximately 3-fold compared to WT, it was reduced to ˜18% by substituting the lysine at position 322 to glutamatic acid (E) (FIG. 14). Similarly, CDC by IgG2-Campath-E430G was reduced to background levels upon introduction of mutation K322E. Also IgG3 and IgG4 isotype variants containing both an E430G and a K322E mutation failed to show CDC lysis above background levels.

These data illustrate that substituting lysine at position 322 into glutamic acid resulted in efficient inhibition of CDC efficacy by IgG1, IgG2, IgG3, and IgG4 isotype variants of IgG1-Campath-E430G.

### Example 19

The Effect of Mutations P329R and K322E on the In Vitro CDC Efficacy of Campath Variants with Different Mutations Inducing Enhanced Fc-Fc Interactions

The effect of mutations P329R and K322E on in vitro CDC efficacy was tested using different Fc-Fc interaction promoting variants of the antibody IgG1-Campath. Different concentrations of purified antibodies (range 0.001-30.0 μg/mL final concentrations) were tested in an in vitro CDC assay on Wien 133 cells with 20% NHS essentially as described in Example 2. The area under the dose-response curves of three experimental replicates was calculated using a log transformed concentration axis with GraphPad Prism 7.02 and normalized relative to cell lysis measured for isotype control antibody IgG1-b12 (0%) and IgG1-Campath (100%).

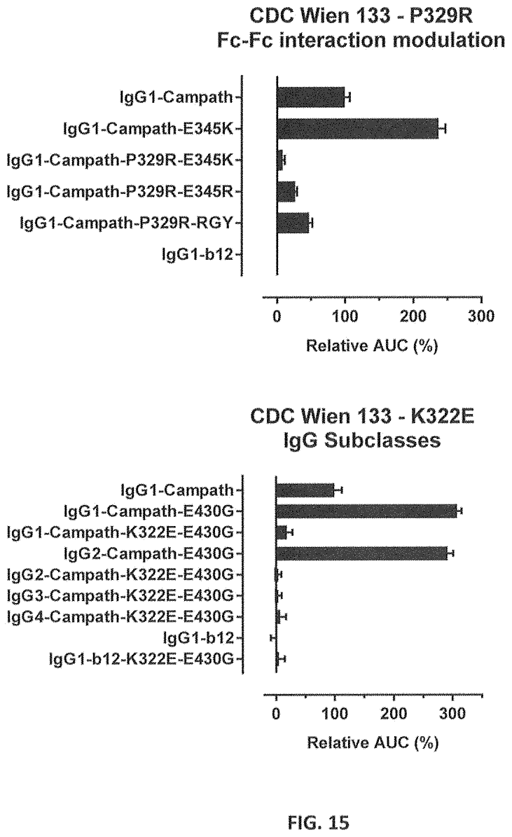

The area under the CDC dose response curve on Wien 133 cells of IgG1-Campath-E345K, containing the Fc-Fc interaction promoting mutation E345K, increased approximately 2.4-fold compared to WT. Substituting the proline at position 329 to arginine (R) limited the CDC to approximately 8%. Furthermore, the introduction of P329R into two other variants E345R and E345R/E430G/S440Y (RGY) with increased Fc-Fc interactions limited CDC activity to levels below that observed for the parental IgG1-Campath antibody (FIG. 15).

Substituting the lysine at position 322 to glutamic acid (E) in antibody IgG1-Campath-E345K decreased the area under the CDC dose response curve from approximately 240% to 24% of that of the parental IgG1-Campath antibody. Introduction of K322E into variant E345R with increased Fc-Fc interactions limited the CDC activity of this variant to 60% of that observed for the parental IgG1-Campath antibody (FIG. 15). However, K322E could not limit CDC of variant RGY to levels below that of IgG1-Campath, in contrast to mutation P329R.

These data suggest that the inhibition of direct C1q binding via mutations P329R or K322E in the C1q binding site can be partially compensated by mutations promoting enhanced Fc-Fc interactions such as E345R and RGY, which promote the formation of multi-valent C1q binding sites in IgG hexamers at the cell surface. Since IgG1-Campath-P329R-RGY showed lower CDC activity than IgG1-Campath-K322E-RGY, P329R appears to be a more potent inhibitor of direct C1q binding than K322E.

In summary, these data illustrate that substituting proline at position 329 into arginine, or lysine at position 322 into glutamic acid, could inhibit the CDC efficacy of IgG1-Campath variants with different Fc-Fc interaction strengths.

### Example 20

The Effect of Mutations P329R, P329D and K322E on the In Vitro CDC Efficacy of Anti-CD20 Antibodies with Enhanced Fc-Fc Interactions

The effect of mutations P329R, P329D and K322E on in vitro CDC efficacy was tested using variants of anti-CD20 antibodies IgG1-11B8 (type II) and IgG1-7D8 (type I) (WO2004/035607). Different concentrations of purified antibodies (range 0.001-30.0 μg/mL final concentrations) were tested in an in vitro CDC assay on Wien 133 cells with 20% NHS essentially as described in Example 2.

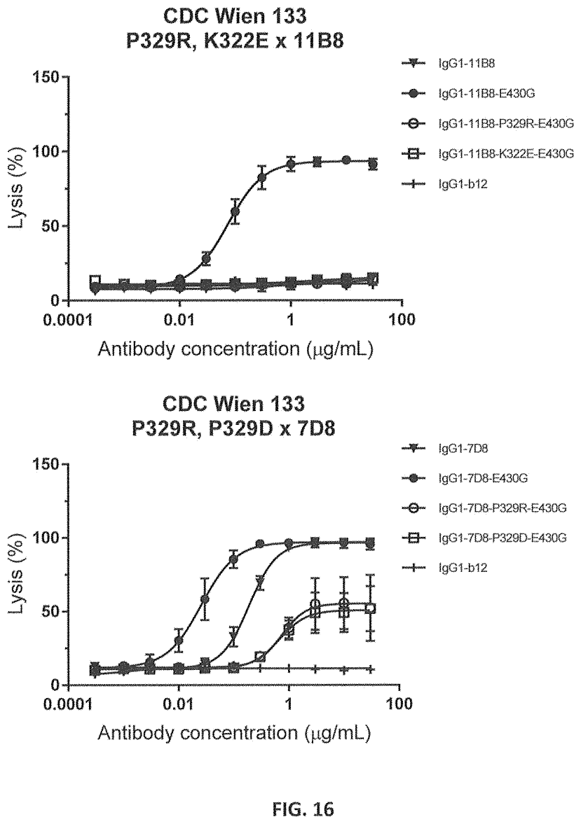

While IgG1-11B8 did not show detectable CDC, introduction of the mutation E430G that induces enhanced Fc-Fc interactions, promoted efficient cell lysis (IgG1-11B8-E430G, FIG. 16). Both mutations P329R and K322E limited the CDC activity of IgG1-11B8-E430G to the background lysis levels observed for non-binding isotype control antibody IgG1 b12.

IgG1-7D8 was capable of inducing CDC of Wien 133 cells, but the CDC efficacy was stimulated by introduction of Fc-Fc interaction enhancing mutation E430G. Introduction of mutation P329R or P329D suppressed CDC activity to levels below that of the wild type parental antibody IgG1-7D8. Without being limited by theory, Fc-region independent accessory CDC mediated through B-cell receptor association, may contribute to the residual CDC detected for IgG1-7D8-P329R-E430G and IgG1-7D8-P329D-E430G, which is typical for type I antibodies against CD20.

These data illustrate that substituting lysine at position 322 into glutamic acid, or proline at position 329 to arginine or aspartic acid, resulted in the inhibition of CDC efficacy of two different anti-CD20 antibodies.

### Example 21

The Effect of K322E or P329X Mutations on the In Vitro FcγR Binding of Anti-CD38 Antibodies with Enhanced Fc-Fc Interactions

Binding of IgG1-005 antibody variants to a monomeric extracellular domain (ECD) of FcγRI and dimeric variants of ECD's of FcγRIIA allotype 131H, FcγRIIA allotype 131R, FcγRIIB, FcγRIIIA allotype 158F, and FcγRIIIA allotype 158V was tested in ELISA assays using purified antibodies.

For detection of binding to FcγRI, 96-well Microlon ELISA plates (Greiner, Germany) were coated overnight at 4° C. with His-tagged FcγRI ECD (1 μg/ml) in PBS, washed and blocked with 200 μL/well PBS/0.2% BSA for 1 h at room temperature (RT). With washings in between incubations, plates were sequentially incubated with 100 μL/well of a dilution series of IgG1-005 antibody variants (0.0013-20 μg/mL in five-fold steps) in PBST/0.2% BSA for 1 h at RT and 100 μL/well of anti-human-KappaLC-HRP (Sigma-Aldrich, A-7164, 1:5.000) in PBST/0.2% BSA for 30 min at RT as detecting antibody for 30 min at RT. Development was performed for circa 15 min with 1 mg/mL 2,2′-azino-bis (3-ethylbenzothiazoline-6-sulfonic acid) (ABTS; Roche, Mannheim, Germany). Reactions were stopped by the addition of 100 μL 2% oxalic acid.

For detection of binding to dimeric FcγR variants, 96-well Microlon ELISA plates (Greiner, Germany) were coated overnight at 4° C. with goat F(ab′)2-anti-human-IgG-F(ab′)2 (Jackson Laboratory, 109-006-097, 1 μg/ml) in PBS, washed and blocked with 200 μL/well PBS/0.2% BSA for 1 h at room temperature (RT). With washings in between incubations, plates were sequentially incubated with 100 μL/well of a dilution series of IgG1-005 antibody variants (0.0013-20 μg/mL in five-fold steps) in PBST/0.2% BSA for 1 h at RT, 100 μL/well of dimeric, His-tagged, C-terminally biotinylated FcγR ECD variants (1 μg/mL) in PBST/0.2% BSA for 1 h at RT, and with 100 μL/well Streptavidin-polyHRP (CLB, M2032, 1:10.000) in PBST/0.2% BSA as detecting antibody for 30 min at RT. Development was performed for circa 10 (IIA-131H, IIA-131R, IIIA-158V), 20 (IIIA-158F), or 30 min (IIB) with 1 mg/mL ABTS (Roche, Mannheim, Germany). Reactions were stopped by the addition of 100 μL 2% oxalic acid.

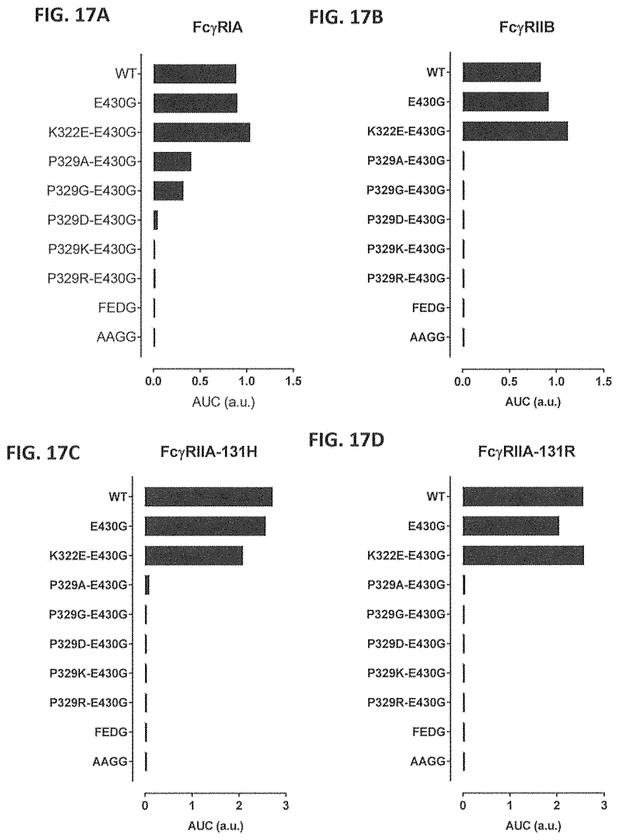

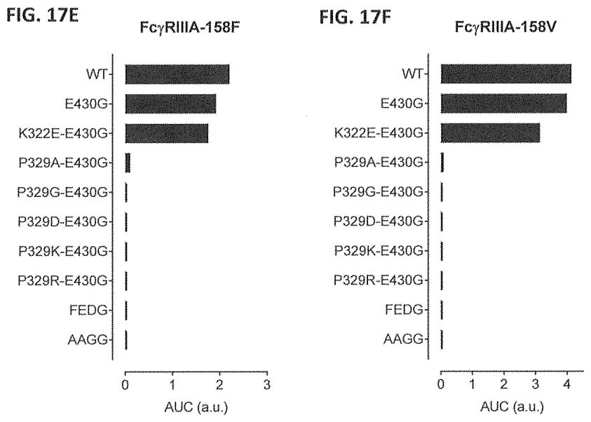

Absorbances were measured at 405 nm in a microplate reader (BioTek, Winooski, Vt.). Log transformed data were analyzed by fitting sigmoidal dose-response curves with variable slope using GraphPad Prism 7.02 software. The area under the dose-response curve was calculated using a log transformed concentration axis.

Whereas K322E potently inhibited CDC by anti-CD38 antibodies containing Fc-Fc enhancing mutations (Examples 3 and 4), this mutation had limited effect on antibody binding to different FcγR variants (FIG. 17), consistent with the preservation of ADCC activity observed in Example 10. In contrast, introducing mutations of P329 (P329A/G/D/K/R) in Fc-Fc enhanced antibody variants reduced binding to FcγRIIA-131H (FIG. 17C), FcγRIIA-131R (FIG. 17D), FcγRIIB (FIG. 17B), FcγRIIIA-158F (FIG. 17E), and FcγRIIIA-158V (FIG. 17F) essentially to background levels, comparable to L234A/L235A/P329G/E430G (AAGG) and L234F/L235E/P329D/E430G (PEDG). Interestingly, antibodies containing different substitutions of P329 differed in their binding to FcγRI (FIG. 17A). While mutations P329A and P329G retained considerable binding to FcγRI, substitutions P329D, P329K and P329R reduced also FcγRI essentially to background levels, comparable to L234A/L235A/P329G/E430G (AAGG) and L234F/L235E/P329D/E430G (PEDG).

In conclusion, variant K322E could potently suppress CDC (Examples 3, 4), but retained binding to all tested FcγR variants. Substitutions P329D, P329K and P329R potently inhibited CDC (Examples 3, 6, 11), but in addition also blocked the binding of Fc-Fc enhanced antibodies to all tested FcγR (FIG. 17). In contrast, Fc-Fc enhanced antibodies containing mutations P329A or P329G retained considerable binding to FcγRI.

### Example 22

The Effect of Mutation P329R on the In Vitro CDC Efficacy of Anti-CD20+Anti-CD52 Antibody Mixtures with Enhanced Fc-Fc Interactions

The effect of mutation P329R on in vitro CDC efficacy was tested using mixtures of variants of anti-CD20 antibody IgG1-11B8 and anti-CD52 antibody IgG1-Campath. Different concentrations of purified antibodies (range 0.001-60.0 μg/mL final concentrations) were tested in an in vitro CDC assay on Wien 133 cells with 20% NHS essentially as described in Example 2. Different mutations were introduced in antibodies IgG1-11B8 and IgG1-Campath: E430G, which induces enhanced Fc-Fc interactions; P329R, which inhibits direct C1q binding to antibodies; and either of the mutations K439E or S440K, which inhibit self Fc-Fc interactions and promote the formation of hetero-hexameric antibody complexes through cross-complementary Fc-Fc interactions. As controls, single antibodies were also mixed 1:1 with non-binding isotype control antibodies IgG1-b12 or IgG1-b12-E430G to enable direct comparison of the concentrations of individual components and mixtures composed thereof. The area under the dose-response curves of three experimental replicates was calculated using a log transformed concentration axis with GraphPad Prism 7.02 and normalized relative to cell lysis measured for isotype control antibody IgG1-b12 (0%) and for the mixture of IgG1-Campath-E430G+IgG1-11B8-E430G (100%).

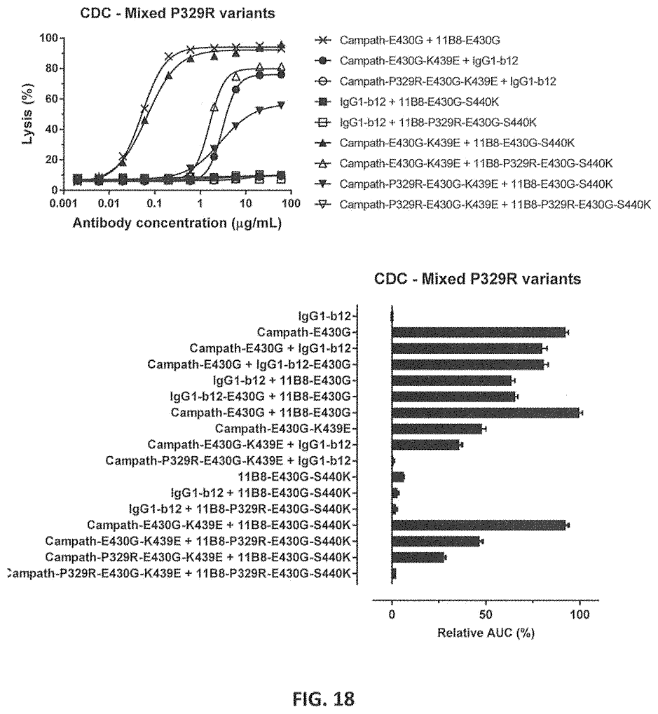

A 1:1 mixture of IgG1-Campath-E430G and IgG1-11B8-E430G promoted efficient cell lysis (FIG. 18). Introduction of mutation K439E decreased the CDC efficacy of IgG1-Campath-E430G, while introducing additional mutation P329R to create IgG1-Campath-P329R-E430G-K439E reduced CDC activity to background level, as defined by the lysis observed for isotype controls IgG1-b12 and IgG1-b12-E430G (FIG. 18). Both single mutation S440K and the double mutation S440K-P329R limited the CDC efficacy of IgG1-11B8-E430G to background level.

Adding IgG1-11B8-E430G-S440K to partially active antibody IgG1-Campath-E430G-K439E restored CDC activity to a level similar to that of IgG1-Campath-E430G+IgG1-11B8-E430G, while adding IgG1-11B8-P329R-E430G-S440K to IgG1-Campath-E430G-K439E resulted in a partial recovery of CDC activity when compared to IgG1-Campath-E430G-K439E. Adding IgG1-11B8-E430G-S440K to IgG1-Campath-P329R-E430G-K439E, both of which failed to show detectable CDC activity, recovered approximately 56% cell lysis at saturating target binding. In contrast, adding IgG1-11B8-P329R-E430G-S440K to IgG1-Campath-P329R-E430G-K439E did not yield CDC activity above background level.

These data show that the P329R mutation improved the selectivity of an IgG-E430G-K439E+IgG-E430G-S440K antibody mixture, by suppressing the single agent activity of one of the two components. Surprisingly, even if both individual components did not show detectable CDC activity, CDC activity was still partially restored for mixtures in which only one of the two antibodies contained the P329R mutation. Without being limited by theory, the avidity of C1q for three unmutated, non P329R-containing binding sites in hetero-hexameric IgG assemblies may be sufficiently high to recover partial CDC activity. In contrast, the loss of all six C1q binding sites, e.g. in mixtures of Abs that both contain P329R mutations, reduced CDC activity to background level.

### Example 23

The Effect of Mutation K322E on the In Vitro CDC Efficacy of Anti-CD20+Anti-CD52 Antibody Mixtures with Enhanced Fc-Fc Interactions

The effect of mutation K322E on in vitro CDC efficacy was tested using mixtures of variants of anti-CD20 antibody IgG1-11B8 and anti-CD52 antibody IgG1-Campath. Different concentrations of purified antibodies (range 0.001-30.0 μg/mL final concentrations) were tested in an in vitro CDC assay on Wien 133 cells with 20% NHS essentially as described in Example 2. Different mutations were introduced in antibodies IgG1-11B8 and IgG1-Campath: E430G, which induces enhanced Fc-Fc interactions; K322E, which inhibits direct C1q binding to antibodies; and either of the mutations K439E or S440K, which inhibit self Fc-Fc interactions and promote the formation of hetero-hexameric antibody complexes through cross-complementary Fc-Fc interactions. The area under the dose-response curves of three experimental replicates was calculated using a log transformed concentration axis with GraphPad Prism 7.02 and normalized relative to cell lysis measured for isotype control antibody IgG1-b12 (0%) and for the mixture of IgG1-Campath-E430G+IgG1-11B8-E430G (100%).

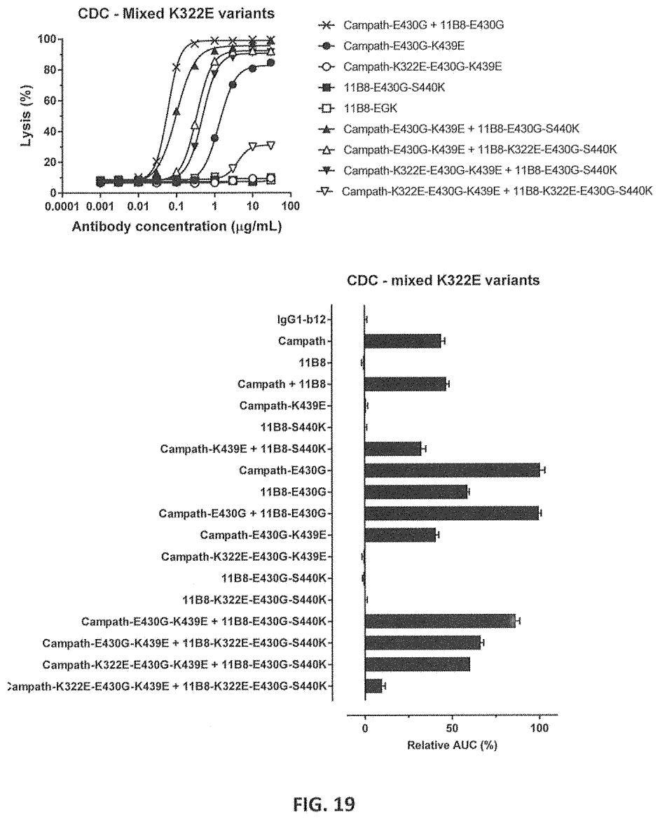

A 1:1 mixture of IgG1-Campath-E430G and IgG1-11B8-E430G promoted efficient cell lysis (FIG. 19). Introduction of mutation K439E decreased the CDC efficacy of IgG1-Campath-E430G, while introducing additional mutation K322E to create IgG1-Campath-K322E-E430G-K439E reduced CDC activity to the background level observed for non-binding control IgG1-b12 (FIG. 19). Both single mutation S440K and the double mutation S440K-K322E limited the CDC efficacy of IgG1-11B8-E430G to background level.

Adding IgG1-11B8-E430G-S440K to partially active antibody IgG1-Campath-E430G-K439E restored CDC activity to a level similar to the maximal level observed for IgG1-Campath-E430G+IgG1-11B8-E430G, while also the combination of IgG1-11B8-K322E-E430G-S440K with IgG1-Campath-E430G-K439E recovered approximately maximal CDC activity. A mixture of IgG1-11B8-E430G-S440K and IgG1-Campath-K322E-E430G-K439E, that both failed to show detectable CDC activity as single agents, recovered cell lysis to approximately 90%. In contrast, adding IgG1-11B8-K322E-E430G-S440K to IgG1-Campath-K322E-E430G-K439E yielded a maximal cell lyis of approximately 31%.

These data illustrate that the introduction of mutation K322E, which inhibits direct C1q binding, could further suppress the CDC activity of individual components in K439E+S440K antibody mixtures. Surprisingly, even if both individual components failed to show detectable CDC activity as single agents, near-maximal cell lysis by CDC could still be restored for mixtures in which only one of the two antibodies contained the K322E mutation.

### Example 24

Selective Killing of Different Cell Lines by Anti-CD20+Anti-CD52 Antibody Mixtures with Enhanced Fc-Fc Interactions

Example 23 demonstrated that specific combinations of Fc-Fc enhanced CD20- and CD52-directed antibodies could selectively lyse Wien 133 target cells at appreciable levels only if both components were simultaneously present, provided each of the antibodies contained either a K439E or an S440K mutation blocking self-oligomerization via Fc-Fc interactions. The selective activity of the mixture compared to its individual components was improved, if direct C1q binding of the anti-CD52 antibody was suppressed by introducing a further K322E mutation (Example 23) or P329R mutation (Example 22). The selective CDC-mediated cell lysis for mixtures of anti-CD20+anti-CD52 antibodies, when compared to their individual components, was tested for seven different cell lines using in vitro CDC assays with 20% NHS essentially as described in Example 2. Different mutations were introduced in antibodies IgG1-11B8 and IgG1-Campath: E430G, which induces enhanced Fc-Fc interactions; K322E, which inhibits direct C1q binding to antibodies; and either of the mutations K439E or S440K, which inhibit self Fc-Fc interactions and promote the formation of hetero-hexameric antibody complexes through cross-complementary Fc-Fc interactions.

In vitro CDC efficacy was tested using mixtures of variants of anti-CD20 antibody IgG1-11B8 and anti-CD52 antibody IgG1-Campath. Final concentrations of 30.0 μg/mL purified antibodies were tested in an in vitro CDC assay with 20% NHS essentially as described in Example 2, on seven human cancer cell lines: Daudi (ATCC #CCL-213), Raji (ATCC #CCL-86), Ramos (ATCC #CRL-1596), REH (DSMZ #ACC22), U266B1 (ATCC #TIB-196), U-698-M (DSMZ #ACC4), and Wien 133 (kindly provided by Dr. Geoff Hale (BioAnaLab Limited, Oxford, UK). Cell lysis was averaged over of three experimental replicates and normalized per cell line relative to the cell lysis measured for isotype control antibody IgG1-b12 (0%) and for IgG1-Campath-E430G (100%, for REH, U266B1, and Wien 133 cells) or IgG1-11B8-E430G (100%, for Daudi, Raji, Ramos, and U-698-M cells), depending on which antibody induced the highest lysis.

The CD52 and CD20 expression at the cell surface of the seven cell lines was determined by indirect immunofluorescence using QIFIKIT (Biocytex, Cat nr CP010). 100,000 cells per well were seeded in polystyrene 96-well round-bottom plates (Greiner Bio-One, Cat nr 650101). The next steps were performed at 4° C. Cells were pelleted by centrifugation for 3 minutes at 300×g and resuspended in 50 μL PBS containing saturating concentrations of 10 μg/mL human monoclonal anti-CD52 antibody IgG1-Campath or anti-CD20 antibody IgG1-11B8. After an incubation of 30 minutes at 4° C., cells were pelleted by centrifugation at 300 g for 3 min and resuspended in 150 μL FACS buffer (PBS+0.1% (w/v) bovine serum albumin (BSA)+0.02% (w/v) sodium azide). Set-up and calibration beads were added to the plate according to the manufacturer's instructions. Cells and beads in parallel were washed two more times with 150 μL FACS buffer and resuspended in 50 μL FITC-conjugated mouse-IgG absorbed goat anti-human IgG (BioCytex). Secondary antibody was incubated for 30 minutes at 4° C. Cells and beads were washed twice with 150 μL FACS buffer and resuspended in 150 μL FACS buffer. Cells were resuspended in fixative (BioCytex) and incubated between 5 and 60 min at 4° C. protected from light. Immunofluorescence was measured on a FACS Canto II (BD Biosciences) by recording 10,000 events within the population of viable cells. The Geometric mean of fluorescence intensity of the calibration beads was used to calculate the calibration curve that was forced to go through zero intensity and zero concentration using GraphPad Prism software (GraphPad Software 7, San Diego, Calif., USA). For each cell line, the antibody binding capacity (ABC), an estimate for the number of antigen molecules expressed on the plasma membrane, was calculated using the Geometric mean fluorescence intensity of the human antibody-stained cells, based on the equation of the calibration curve (interpolation of unknowns from the standard curve, using GraphPad Software), followed by subtraction of the background determined for wells incubated without primary antibody. The number of molecules expressed as ABC was averaged over two independent experiments and is summarized in table 4, ordered by CD52 expression.

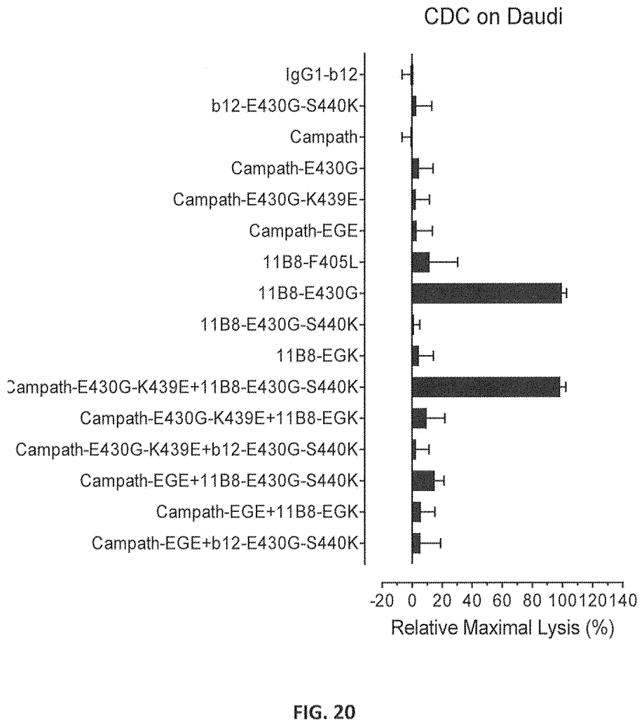

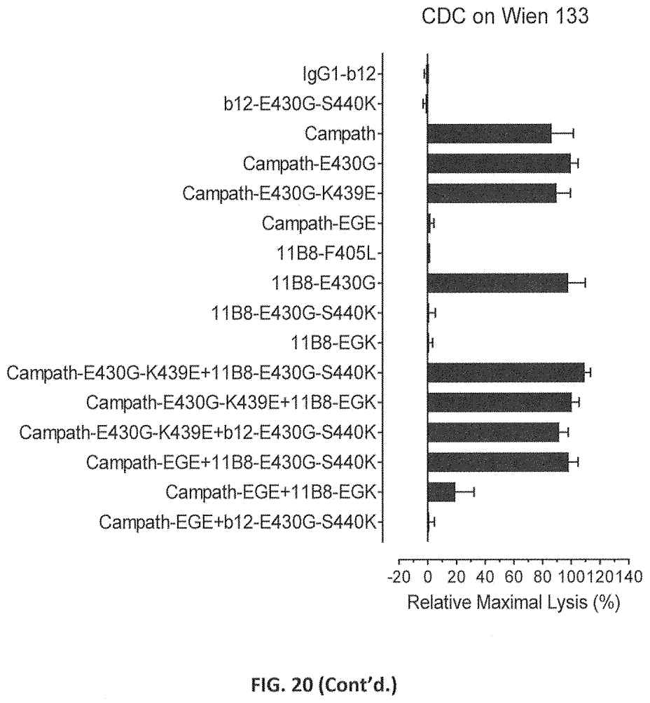

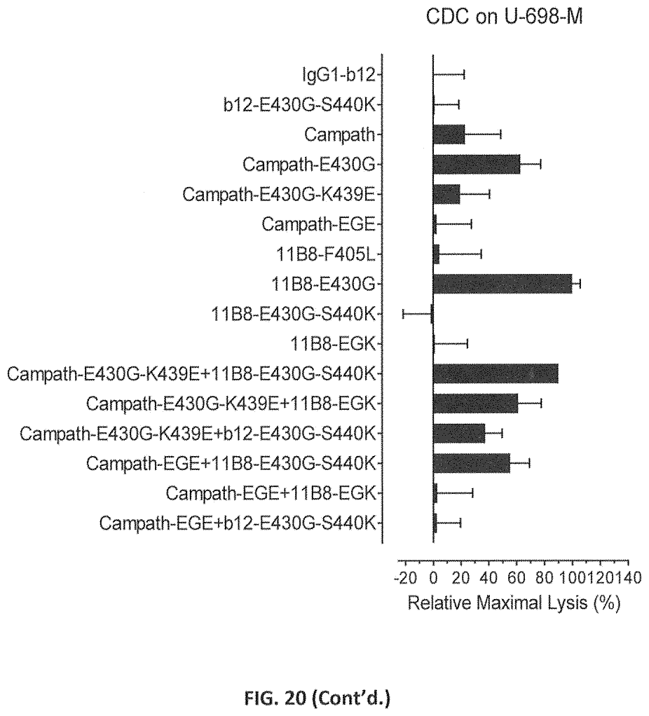

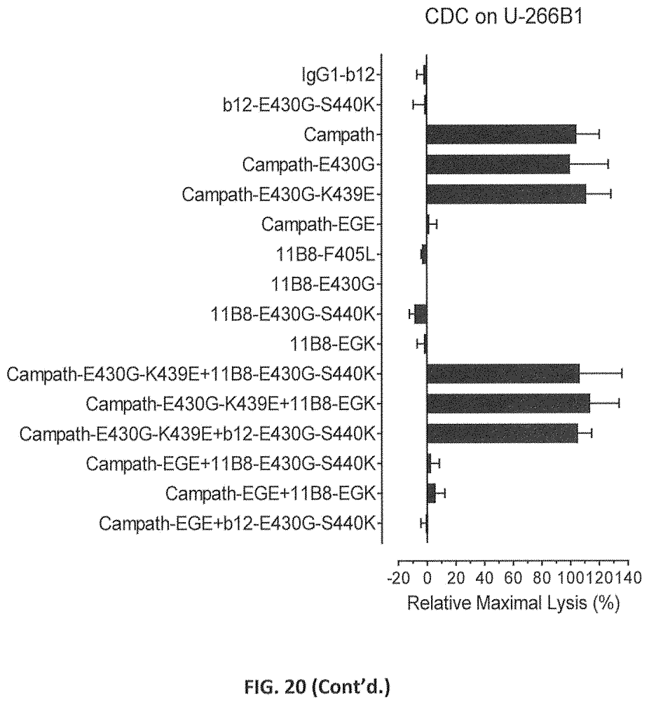

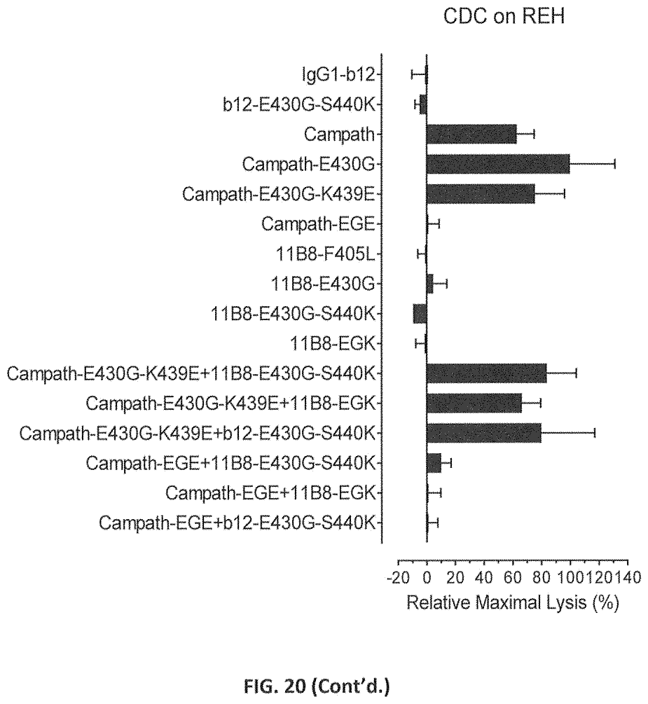

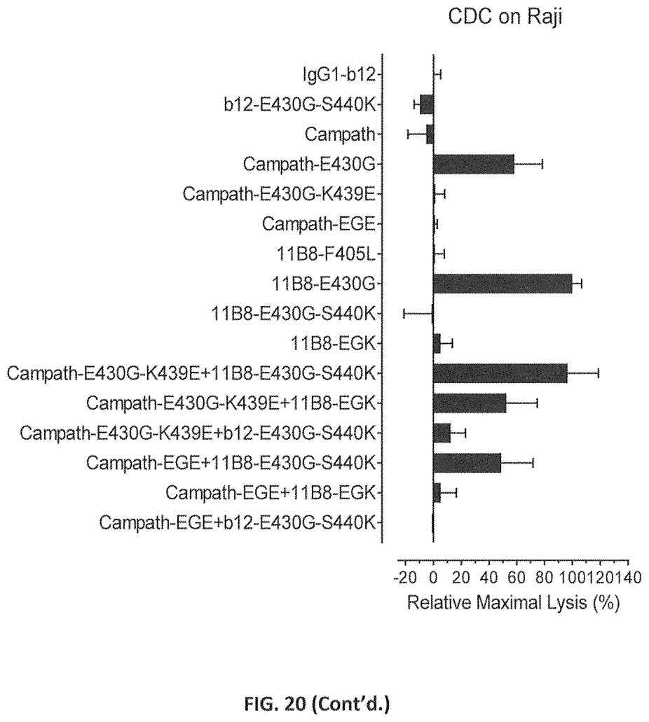

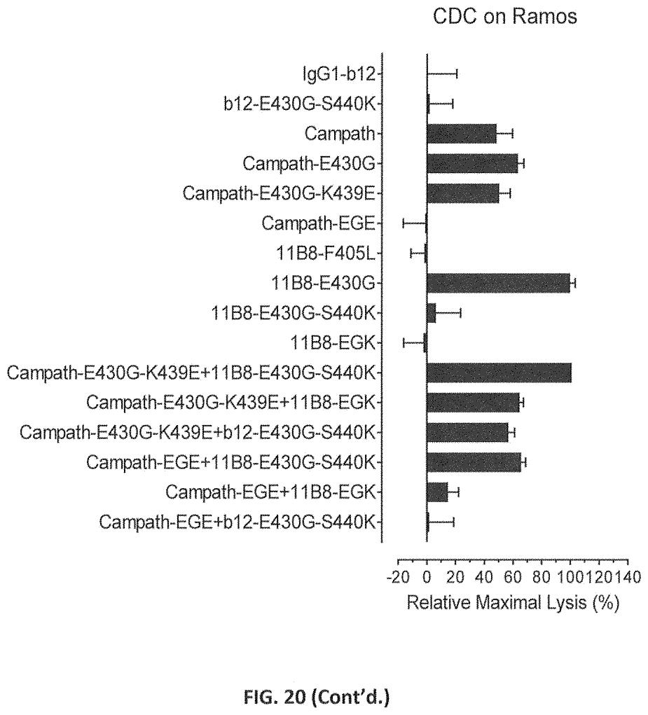

The lysis induced by IgG1-Campath-E430G or IgG1-11B8-E430G varied with the target expression (FIG. 20): whereas low CD20 expressing U-266B1 and REH cells were resilient to lysis by anti-CD20 Ab IgG1-11B8-E430G, they were sensitive to anti-CD52 Ab IgG1-Campath-E430G. In contrast, the low CD52 expressing Daudi cells were resilient to IgG1-Campath-E430G, but sensitive to IgG1-11B8-E430G.

To achieve selective formation of Ab hexamers only when both CD52 and CD20 are present at the cell surface, additional K439E, S440K and/or K322E mutations were introduced into IgG1-Campath-E430G or IgG1-11B8 variants to suppress single agent activity. IgG1-Campath-E430G-K439E showed reduced single agent activity on the relatively low CD52 expressing cell lines U-698-M and Raji compared to IgG1-Campath-E430G, but displayed maximal lysis levels similar to IgG1-Campath-E430G for the high CD52 expressing cell lines U-26661, Wien 133, Ramos, and REH. When C1q binding was reduced by introducing an additional K322E mutation creating IgG1-Campath-K322E-E430G-K439E (Campath-EGE), single agent activity was eliminated for all seven cell line tested. The activity of IgG1-11B8-E430G could already be blocked using only an S440K mutation for all cell lines sensitive to IgG1-11B8-E430G mediated lysis; IgG1-11B8-EGK, containing K322E, E430G and S440K mutations also displayed single agent activity comparable to background defined by non-binding IgG1-b12.

When IgG1-Campath-E430G-K439E was mixed with IgG1-11B8-E430G-S440K, all seven cell lines were lysed, illustrating absence of selectivity. In stark contrast, a mixture of IgG1-Campath-K322E-E430G-K439E (Campath EGE) and IgG1-11B8-E430G-S440K showed selective lysis of only those cell lines that displayed surface expression of both CD20 and CD52 at levels above 20,000 copies per cell, i.e. Wien 133, Ramos, U-698-M, and Raji. IgG1-Campath-EGE activity could not be restored using an IgG1-b12-E430G-S440K control antibody that is not recruited to the cell surface. In contrast, U-266B1, REH, and Daudi were not lysed due to the low expression of either CD20 or CD52. This suggests that the recruitment of C1q by IgG1-Campath-EGE is dependent on its hetero-oligomerization with IgG1-11B8-E430G-S440K. Indeed, CD20 antibody IgG1-11B8-K322E-E430G-S440K (IgG1-11B8-EGK), also containing the K322E mutation reducing C1q binding, could not restore efficient cell lysis when added to IgG1-Campath-EGE.

In conclusion, selective killing of cells expressing appreciable levels of both CD20 and CD52 could be achieved using a mixture of antibodies IgG1-Campath-K322E-E430G-K439E and IgG1-11B8-E430G-S440K; in contrast, this mixture displayed background lysis levels of cell lines that expressed either CD20 or CD52 at levels <20,000 copies Per cell.

Table 4 summarizes the cell surface expression of CD52 and CD20 of different cell lines expressed as the number of specific antibody binding units per cell, determined using QIFIKIT.

| Molecules/cell | U-266B1 | Wien 133 | Ramos | REH | U-698-M | Raji | Daudi |
|---|---|---|---|---|---|---|---|
| Campath/CD52 | 1455918 | 332343 | 178794 | 135088 | 93651 | 85514 | 7275 |
| 11B8/CD20 | 18544 | 101937 | 81198 | 13031 | 70047 | 115310 | 110004 |

### Example 25

The Effect of K322E or P329R Mutations on the Activation of OX40 on Jurkat Cells by Anti-OX40 Antibodies with Enhanced Fc-Fc Interactions

The crosslinking of OX40/CD134 receptors by OX40 ligand can induce the proliferation of T-cells expressing the OX40 receptor (Gramaglia, I., Weinberg, A. D., Lemon, M., and Croft, M. (1998) Ox-40 ligand: a potent costimulatory molecule for sustaining primary CD4 T cell responses. J. Immunol. 161, 6510-6517). The effect of mutations K322E or P329R on OX40/CD134 signaling was tested using different variants of the anti-OX40 antibody IgG1-SF2 (U. S. Patent 2014/0377284) using the OX40 Bioassay Kit (Promega, #CS197704) essentially according to the instructions supplied by the manufacturer. Thaw-and-Use GloResponse NFκB-luc2/OX40 Jurkat cells (Promega, #CS197704), which stably express human OX40 and a luciferase reporter gene downstream of an NFAT response element, express luciferase upon OX40 activation. 25 μL freshly thawed cells were incubated overnight in 96-well white F-bottom Optiplates (Perkin Elmer, #6005299) in 25 μL RPMI 1640 medium (Promega, #G708A) in the presence of 8% serum from different sources detailed below. The following day, 2.5 μg/mL (end concentration) antibodies or 1.5 μg/mL (end concentration) of purified, recombinant OX40 ligand (Biolegend, #555704) were added to the cells in medium to an end volume of 80 μL. Cells were incubated for a further 5 hours prior to addition of the Bio-Glo Reagent (Promega, #CS197704). After 5-10 min incubation at ambient temperature, luminescence was recorded using an Envision MultiLabel Plate reader. Serum sources compared were Fetal Bovine Serum (FBS, Promega Ref. 3121A), FBS heat-inactivated for 30 min at 56° C., human C1q-depleted serum (Quidel, #A509), human C1q-depleted serum supplemented with human recombinant C1q (1.0 μg/mL end concentration; Quidel, #A400), or normal human serum (NHS, Sanquin, Ref. M0008AC).

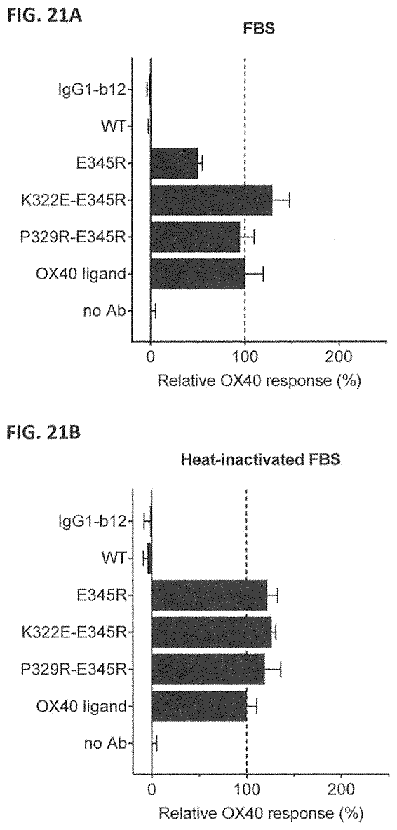

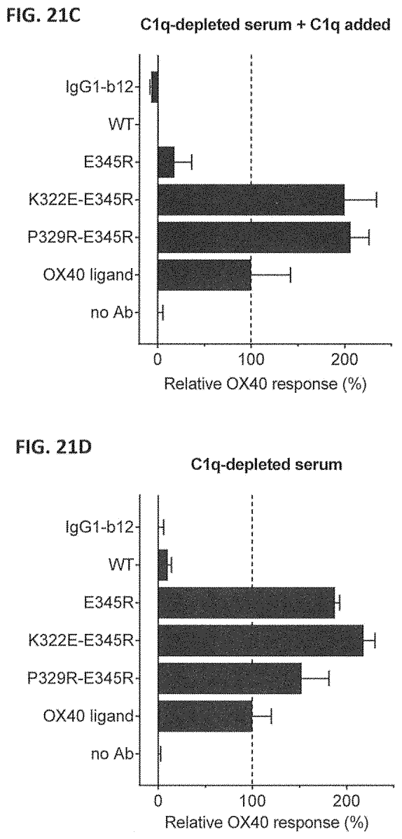

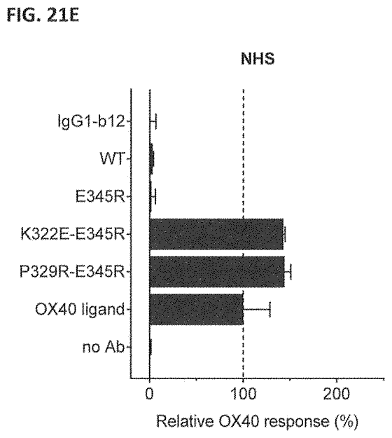

Recombinant OX40 ligand, which was used as a positive control in the OX40 response assay, induced clear response signals relative to the non-binding negative control antibody IgG1-b12 (FIG. 21). OX40 responses induced by test compounds were normalized relative to incubations without antibody (0%) and incubations with OX40 ligand (100%). Wild type anti-OX40 antibody IgG1-SF2 induced OX40 response levels essentially similar to the negative control antibody IgG1-b12, regardless the source of serum. In contrast, IgG1-SF2 variants that contained only the E345R mutation, which induces Fc-Fc interactions between antibodies after cell surface binding, induced varying OX40 responses, which exceeded the level of OX40 ligand (>100%) when C1q was inactivated or depleted (FIG. 21B/D).

Introduction of the K322E or P329R mutation in IgG1-SF2-E345R did not significantly affect the OX40 response in absence of active complement, i.e. in heat-inactivated FBS (FIG. 21B) and in C1q-depleted serum (FIG. 21D). Surprisingly, introduction of the K322E or P329R mutation in IgG1-SF2-E345R resulted in enhanced OX40 activation in the presence of active complement, i.e. in non-heat-inactivated FBS (FIG. 21A), human C1q-depleted serum in the presence of 1.0 μg/mL human recombinant C1q (FIG. 21C) and in NHS (FIG. 21E).

The surprising observations described in this example could, without being limited by theory, possibly be explained by a difference in C1q binding and complement-dependent cytotoxicity (CDC): when no active C1q is present (FIG. 21B/D), IgG1-5F2-E345R can already cluster optimally to induce OX40 activation, and in this case, introduction of the C1q inhibiting mutation K322E or P329R has no effect. In contrast, optimal OX40 activation by IgG1-SF2-E345R is hampered when active C1q is present (FIG. 21A/C/E), and introduction of the C1q inhibiting mutation K322E or P329R results in restoration of optimal OX40 activation, comparable to the activity in absence of active C1q. The reduction of luciferase activity observed with IgG1-5F2-E345R in the presence of active C1q, when compared to the luciferase activity recorded in the absence of C1q, could be explained by CDC activity, as the high OX40 expression on the Jurkat reporter cell line could sensitize the cells to CDC by the IgG1-SF2-E345R antibody with the E345R Fc-Fc enhancing mutation. Introduction of the additional mutation K322E or P329R, which both can strongly reduce C1q binding and CDC activity of antibodies containing an Fc-Fc-enhancing mutation (described in Example 3; FIG. 2), could than effectively block this CDC activity and therefore allow for optimal OX40 response induction, also in presence of active complement.

In summary, strong OX40 responses exceeding those induced by recombinant OX40 ligand were observed for anti-OX40 antibodies that contained both an Fc-Fc-enhancing E345R mutation, and a C1q-binding inhibiting mutation K322E or P329R, both in the presence or absence of active complement. In contrast, wild type anti-OX40 antibody IgG1-SF2 failed to induce detectable OX40 responses under these assay conditions, and antibody IgG1-SF2-E345R only allowed for maximal OX40 responses under conditions where complement was inactive. In conclusion, the combination of Fc-Fc-enhancing mutations and C1q-inhibiting mutations K322E or P329R yielded surprisingly potent OX40-agonistic antibodies, which may maximize T-cell proliferation under physiologically relevant serum conditions.

### EQUIVALENTS

Those skilled in the art will recognize, or be able to ascertain using no more than routine experimentation, many equivalents of the specific embodiments of the invention described herein. Such equivalents are intended to be encompassed by the following claims. Any and all combinations of embodiments disclosed in dependent claims are also contemplated to be within the scope of the invention.

1. A polypeptide comprising an Fc region of a human IgG and an antigen binding region, wherein the Fc region comprises a CH2 domain and a CH3 domain, said Fc region comprising a (i) first mutation and a (ii) second mutation corresponding to the following amino acid positions in human IgG1 according to EU numbering:
i. first mutation at E430, E345 or S440, with the proviso that the mutation in S440 is S440Y or S440W; and
ii. second mutation at K322 or P329.

2. The polypeptide according to claim 1, wherein the first mutation is selected from the group consisting of: E430G, E345K, E430S, E430F, E430T, E345Q, E345R, E345Y, S440W and S440Y.

3. (canceled)

4. The polypeptide according to claim 1, wherein the second mutation is selected from the group consisting of: K322E, K322D, K322N, P329H, P329K, P329R, P329D, P329E, P329F, P329G, P329I, P329L, P329M, P329N, P329Q, P329S, P329T, P329V, P329W P329A and P329Y.

5-8. (canceled)

9. The polypeptide according to claim 1, wherein the Fc region comprises a further mutation in the CH3 domain at position K439, or if the first mutation is not at position S440, then the further mutation may be at position S440.

10. The polypeptide according to claim 9, wherein the further mutation is selected from S440K or K439E.

11. The polypeptide according to claim 1, wherein the first mutation is E345R and the second mutation is P329R.

12. The polypeptide according to claim 1, wherein the polypeptide has an Fc effector function which is decreased by at least 20% compared to a parent polypeptide which is identical to the polypeptide with the same first mutation but without the second mutation.

13. (canceled)

14. The polypeptide according to claim 12, wherein the Fc effector function is selected from the following group; complement dependent cytotoxicity (CDC), complement dependent cell-mediated cytotoxicity (CDCC), complement activation, antibody-dependent cell-mediated cytotoxicity (ADCC), antibody dependent cell-mediated phagocytosis (ADCP), C1q binding and FcγR binding.

15. The polypeptide according to claim 1, wherein the polypeptide is an antibody, monospecific antibody, bispecific antibody or multispecific antibody.

16. The polypeptide according to claim 1, wherein the Fc region is a human IgG1, IgG2, IgG3, IgG4, IgE, IgD, IgM, IgA isotype or a mixed isotype.

17. (canceled)

18. The polypeptide according to claim 1, wherein the polypeptide is a human antibody, humanized antibody or chimeric antibody.

19. The polypeptide according to claim 1, wherein the antigen binding region binds to a member of the tumor necrosis factor receptor superfamily (TNFR-SF).

20. (canceled)

21. The polypeptide according to claim 19, wherein the member of the TNFR-SF is selected from the group consisting of: FAS, DR4, DR5, TNFR1, DR6, DR3, EDAR, NGFR, OX40, CD40, CD30, CD27, 4-1BB, RANK, TACI, BLySR, BCMA, RELT and GITR.

22. (canceled)

23. A method of decreasing an Fc effector function of a polypeptide comprising an Fc region of a human immunoglobulin and an antigen binding region, wherein the Fc region comprises a CH2 and CH3 domain, said Fc region comprising a (i) first mutation corresponding to the following positions in human IgG1 according to EU numbering: E430, E345 or S440, which method comprises introducing a (ii) second mutation corresponding to the following positions in human IgG1 according to EU numbering: K322 or P329.

24. The method according to claim 23, wherein the first mutation is selected from the group consisting of: E430G, E345K, E430S, E430F, E430T, E345Q, E345R, E345Y, S440W and S440Y.

25. (canceled)

26. The method according to claim 23, wherein the second mutation is selected from the group consisting of: K322E, K322D, K322N, P329H, P329K, P329R, P329D, P329E, P329F, P329G, P329I, P329L, P329M, P329N, P329Q, P329S, P329T, P329V, P329W, P329A and P329Y.

27-28. (canceled)

29. The method according to claim 23, wherein the Fc region comprises a further mutation in the CH3 domain corresponding to one of the following positions in human IgG1 according to EU numbering: S440 or K439.

30. (canceled)

31. The method according to claim 23, wherein the first mutation is E345R and the second mutation is P329R.

32-33. (canceled)

34. A composition comprising at least one polypeptide according to claim 1.

35. A composition comprising at least one polypeptide according to claim 11.

36. (canceled)

37. The composition according to claim 34, which comprises a first polypeptide comprising a first antigen-binding region and a first Fc region, a second polypeptide or antibody comprising second antigen-binding region and a second Fc region, wherein the first and second Fc region comprises (i) a first mutation, (ii) a second mutation, and (iii) a further mutation, wherein the mutations correspond to the following amino acid positions in human IgG1, according to EU numbering:
(i) a first mutation E430, E345 or S440, with the proviso that the mutation in S440 is S440Y or S440W;
(ii) a second mutation at E322 or P329;
(iii) a further mutation at K439 or S440, with the proviso that if the further mutation is at S440 then the first mutation is not at S440, with the proviso that the first and second Fc region does not comprise a further mutation in the same amino acid position.

38. The composition according to claim 34, wherein said first polypeptide and said second polypeptide bind different epitopes on one or more members of the TNFR-SF with an intracellular death domain selected from the group consisting of: TNFR1, FAS, DR3, DR4, DR5, DR6, NGFR and EDAR, or wherein said first polypeptide and said second polypeptide bind different epitopes on one or more members of the TNFR-SF without an intracellular death domain selected from the group consisting of: OX40, CD40, CD30, CD27, 4-1BB, RANK, TACI, BLySR, BCMA, RELT and GITR.

39-45. (canceled)

46. A method of treating an individual having a disease comprising administering to said individual an effective amount of a polypeptide according to claim 1.

47. The method according to claim 46, wherein the disease is selected from the group consisting of: cancer, autoimmune disease, inflammatory disease and infectious disease.

48. The method according to claim 46, comprising further administering an additional therapeutic agent.

49. The method according to claim 48, wherein the additional therapeutic agent is one or more anti-cancer agent(s) selected from the group consisting of chemotherapeutics, kinase inhibitors, apoptosis-modulating agents, RAS inhibitors, proteasome inhibitors, histone deacetylase inhibitors, nutraceuticals, cytokines, antibodies or antibody mimetics, and antibody-drug conjugates.

50. A kit of parts comprising a polypeptide according to claim 1, or a composition comprising the polypeptide, and instructions for use, wherein said polypeptide or composition is in one or more containers such as vials.

51-53. (canceled)
